(19) United States
(12) Patent Application Publication
(10) Pub. No.: US 2025/0049866 A1
(43) Pub. Date: Feb. 13, 2025

(54) CANCER IMMUNOTHERAPY USING VIRUS PARTICLES

(71) Applicant: CASE WESTERN RESERVE UNIVERSITY, Cleveland, OH (US)

(72) Inventors: Nicole F. Steinmetz, San Diego, CA (US); Karin L. Lee, Cleveland, OH (US)

(21) Appl. No.: 18/785,211

(22) Filed: Jul. 26, 2024

Related U.S. Application Data
(63) Continuation-in-part of application No. 18/115,066, filed on Feb. 28, 2023, now Pat. No. 12,070,478.
Application No. 18/115,066 is a continuation of application No. 16/492,884, filed on Sep. 10, 2019, now Pat. No. 11,590,183.
Application No. 16/492,884 is a continuation-in-part of application No. 15/589,677, filed on May 8, 2017, now Pat. No. 10,639,363.
(63) Continuation-in-part of application No. 17/369,405, filed on Jul. 7, 2021.
Application No. 17/369,405 is a continuation-in-part of application No. 16/612,214, filed on Nov. 8, 2019, now Pat. No. 11,617,787.
Application No. 16/612,214 is a continuation-in-part of application No. 15/589,677, filed on May 8, 2017, now Pat. No. 10,639,363.
Application No. 15/589,677 is a continuation-in-part of application No. PCT/US2015/059675, filed on Nov. 9, 2015.
(60) Provisional application No. 62/469,869, filed on Mar. 10, 2017.
(60) Provisional application No. 63/049,434, filed on Jul. 8, 2020.
(60) Provisional application No. 62/076,543, filed on Nov. 7, 2014.
(60) Provisional application No. 62/107,617, filed on Jan. 26, 2015.
(60) Provisional application No. 62/159,389, filed on May 11, 2015.
(60) Provisional application No. 62/364,997, filed on Jul. 21, 2016.

(51) Int. Cl. A61K 35/76; A61K 31/704; A61K 39/12; A61K 39/39; A61K 45/06; A61P 35/00

(52) U.S. Cl. CPC A61K 35/76; A61K 31/704; A61K 39/12; A61K 39/39; A61P 35/00; A61K 45/06; C12N 2770/26011; C12N 2770/26023; C12N 2770/26033; C12N 2770/26042; C12N 2770/40023

(57) ABSTRACT

An intratumoral immunotherapy for treating cancer in a subject in need includes a therapeutically effective amounts of cowpea mosaic virus (CPMV), CPMV virus-like particles, potato virus X (PVX), and/or PVX virus-like particles formulated for in situ administration to cancer of the subject, wherein the therapeutically effective amount of CPMV, CPMV virus-like particles, PVX, and/or PVX virus-like particles is an amount effective to provide a durable and systemic anticancer response against cancer metastasis and recurrence in the subject, and the CPMV, CPMV virus-like particles, PVX, and/or PVX virus-like particles are not used as a vehicle for drug or antigen delivery.

### RELATED APPLICATION

This application is a Continuation-in-Part of U.S. patent application Ser. No. 18/115,066, filed Feb. 28, 2023, which is a Continuation of U.S. patent application Ser. No. 16/492,884, filed Sep. 10, 2019 (Now U.S. Pat. No. 11,590,183), which is a national phase of PCT/US2018/022023, filed Mar. 18, 2018, which claims priority from U.S. Provisional Application No. 62/469,869, filed Mar. 10, 2017. PCT/US2018/022023 is also a Continuation-in-Part of Ser. No. 15/589,677 (now U.S. Pat. No. 10,639,363). This application is also a Continuation-in-Part of U.S. patent application Ser. No. 17/369,405, filed Jul. 7, 2021, which claims priority from 63/049,434, filed Jul. 8, 2020, patent application Ser. No. 17/369,405 is a Continuation-in-Part of U.S. patent application Ser. No. 16/612,214 (now U.S. Pat. No. 11,617,787) which is a national phase of PCT/US2018/031661, filed May 8, 2018, which is a Continuation-in-Part of U.S. patent application Ser. No. 15/589,677, filed May 8, 2017 (now U.S. Pat. No. 10,639,363), which is a Continuation-in-Part of PCT/US2015/059675, filed Nov. 9, 2015, which claims priority from U.S. Provisional Patent Application No. 62/076,543, filed Nov. 7, 2014, U.S. Provisional Patent Application Ser. No. 62/107,617, filed Jan. 26, 2015, and U.S. Provisional Patent Application Ser. No. 62/159,389, filed May 11, 2015 Ser. No. 15/589,677, filed May 8, 2017, also claims priority to U.S. Provisional Application No. 63/364,997, filed Jul. 21, 2016, the subject matter of all above applications are incorporated herein by reference in their entirety.

### GOVERNMENT FUNDING

This invention was made with government support under CA148052 and EB007509 awarded by The National Institutes of Health. The government has certain rights in the invention.

This invention was made with support under 128319-RSG-15-144-01CDD awarded by the American Cancer Society.

### BACKGROUND

Regardless of tissue of origin, metastatic cancers uniformly carry poor prognoses. Conventional chemo-and radiotherapy are largely ineffective for late stage disease. The emerging field of tumor immunology offers new therapeutic options. Novel therapeutics that seek to induce anti-tumor immunity, such as immune checkpoint inhibitors, chimeric antigen receptor cell therapies, and tumor-associated antigen cancer vaccines show promise, but the development of immunotherapy for cancer is in an early stage. Each cancer type is unique but many solid tumors metastasize to the lungs. An option with limited exploration is direct application of immunostimulatory reagents into the suspected metastatic site or an identified tumor. This approach, in situ vaccination, can modulate the local microenvironment and, like therapies such as T cell checkpoint blocking antibodies, can relieve immunosuppression and potentiate anti-tumor immunity against antigens expressed by the tumor.

Research into nanoparticles as cancer therapies has focused on them as a delivery platform: the loading of particles with tumor-associated antigen and immune agonists for the stimulation of anti-tumor immunity, or the loading of particles with pre-existing conventional chemotherapeutic drugs for delivery to tumors as a means to reduce toxicity. Sheen et al., Wiley Interdiscip Rev Nanomed Nanobiotechnol., 6(5):496-505 (2014). However, the tendency of nanoparticles to interact with and to be ingested by innate immune cells gives them potential as immunostimulatory, immunoregulatory and immunostimulatory agents if they modulate the characteristics of the ingesting innate immune population.

Virus-like particles (VLPs) refer to the spontaneous organization of coat proteins into the three dimensional capsid structure of a particular virus. Like active viruses, these particles are in the 20-500 nm size range. VLPs have already been deployed as antigen components of antiviral vaccines against infectious counterpart viruses hepatitis B (Halperin et al., Vaccine, 30(15):2556-63 (2012)) and human papilloma virus (Moreira et al., Hum Vaccin., 7(7):768-75 (2011)). By preventing infection with viruses that cause cancer, vaccines utilizing VLPs are currently contributing to reductions in cancer incidence.

Recent studies have demonstrated that VLP therapeutic efficacy extends beyond the specific antigen array that they carry and that they may possess inherent immunogenic properties that can stimulate immune responses against infectious agents that do not carry any antigen included in the VLP. Rynda-Apple et al., Nanomed., 9(12):1857-68 (2014)). VLPs have shown the ability to induce protective immune responses in the respiratory tract in mouse models of infectious diseases of the lungs. VLP treatment protected mice from bacterial pneumonia caused by methicillin-resistant Staphylococcus aureus (MRSA) (Rynda-Apple et al., Am J Pathol., 181(1):196-210 (2012)) and Coxiella burnetii (Wiley et al., PLOS ONE., 4(9):e7142 (2009)). VLPs have also been shown to protect mice in various influenza models. Patterson et al., ACS Nano., 7(4):3036-44 (2013); Richert et al., Eur J Immunol., 44(2):397-408 (2014). Protective immunity in these models was associated with recruitment, activation, and increased antigen-processing capabilities, formation of inducible bronchus-associated lymphoid tissue (iBALTs), and stimulation of CD4⁺ T and B lymphocytes and CD8⁺ T cells. It is important to note that these studies reported robust induction of both innate and adaptive immunity and that the VLPs utilized were not antigenically related to the infectious agents, yet appeared to exert their therapeutic effect via the inherent immunomodulatory nature of the particles.

### SUMMARY

Embodiments described herein relate to methods of treating cancer in a subject in need thereof by administering in situ to cancer of the subject a therapeutically effective amount of a virus or virus-like particle. The virus or virus-like particle can be nonreplicating and noninfectious in the subject to avoid infection of the subject. In some embodiments, the in situ administration of the virus or virus-like particle can be proximal to a tumor or directly to the tumor site to provide a high local concentration of the virus or virus-like particle in the tumor microenvironment. The method represents a type of in situ vaccination, in which application of an immunostimulatory reagent directly to the tumor modifies the tumor microenvironment so that the immune system is able to respond to the tumor.

It was found that plant virus and virus-like particles and their unique therapeutic features, originally observed in lung infectious disease models, could be utilized for a new application: treating cancer, such as lung cancer. Primary lung cancer is the second most common cancer in the United States, behind only breast cancer. Additionally, most other majors cancers frequently metastasize to the lung, including breast, bladder, colon, kidney, melanoma, and prostate. Their research focused on melanoma due to its increasing incidence in the US and the poor prognosis for metastatic disease, which currently has a 5-year survival of below 10%. Flaherty et al., N Engl J Med., 363 (9):809-19 (2010).

In some embodiments, the virus or virus-like particle (VLP) can include CPMV or eCPMV VLPs, which does not carry any nucleic acids, or potato virus X (PVX) or PVX VLPs. These plant viruses or VLPs can be nonreplicative and can be regarded as safe from a human health and agricultural perspective. In planta production prevents endotoxin contamination that may be a byproduct of other VLP systems derived from E. coli. The viruses or VLPs are scalable, stable over a range of temperatures (4-60° C.) and solvent:buffer mixtures. In situ vaccination or administration of CPMV and/or PVX virus or VLPs alone or in combination with a chemotherapeutic in a model of metastatic lung melanoma as well as dermal melanoma and other cancers (breast, colon, ovarian, lymphoma), was found to have striking efficacy in treating the cancer.

The in situ vaccination approach does not rely on the viruses or virus-like particles as a vehicle for drug or antigen delivery, but rather on their inherent immunogenicity. This immunogenicity appears to be uniquely potent when the virus or virus-like particle are inhaled or when administered through intratumoral administration into solid tumors or as IP administration when treating disseminated, metastatic cancer. For treatment of lung tumors, the virus or virus-like particles can be intratracheally injected into mice with established lung tumors and this immunostimulatory treatment results in the rejection of those tumors and systemic immunity that prevents growth of distal tumors. The virus or virus-like particle described herein (e.g., CPMV or PVX virus or virus-like particles) alone are able to stimulate systemic anti-tumor immunity. The virus or virus-like particles can potentially render the lung microenvironment inhospitable to tumor cell seeding or continued growth.

### BRIEF DESCRIPTION OF THE FIGURES

FIGS. 1A and 1B provides bar graphs showing eCPMV nanoparticles are inherently immunogenic. (A) Bone marrow-derived dendritic cells (BMDCs) exposed to eCPMV produce elevated levels of pro-inflammatory cytokines in vitro. (B) Thioglycollate-elicited primary macrophages also secrete significantly elevated levels of the same panel of cytokines. Both cell types were cultured for 24 hr with 20 μg eCPMV (dark gray bars) and cytokine levels were analyzed using a multiplexed luminex array.

FIG. 2A-2D provides graphs showing eCPMV inhalation induces dramatic changes in immune cell composition and cytokine/chemokine milieu in B16F10 lung tumor-bearing mice. (A) Representative FACS plots pre-gated on live CD45⁺ cells of non-tumor-bearing mice treated with PBS (top left) or eCPMV (top right) and B16F10 lung tumor-bearing mice treated with PBS (bottom left) or eCPMV (bottom right). B16F10 mice were treated on day 7 post-B16F10 IV injection. Lungs were harvested 24 hr after intratracheal injection of PBS or 100 ug eCPMV. Labeling indicates (i) quiescent neutrophils, (ii) alveolar macrophages, (iii) monocytic MDSCs, (iv) granulocytic MDSCs, (v) tumor-infiltrating neutrophils, and (vi) activated neutrophils. Numbers beside circled groups is % of CD45⁺ cells. Arrows indicate TINs (blue) and CD11b⁺ activated neutrophils (red). Gating strategies available in supplemental data. (B) Changes in innate cell subsets induced by eCPMV inhalation are quantified as a percentage of CD45⁺ cells (top) and total number of cells (bottom) as presented in panel (A). (C) Representative histograms for TINs, activated neutrophils, alveolar macrophages, and monocytic MDSCs indicating uptake of Alexa488-labeled CPMV, class-II, and CD86 activation markers. (D) Lungs of B16F10 lung tumor-bearing mice exhibited elevated levels of pro-inflammatory cytokines and chemoattractants when treated with eCPMV as in panel (A).

FIGS. 3A-3D provide graphs and images showing eCPMV inhalation prevents formation of B16F10 metastatic-like lung tumors. (A) Schematic of experimental design. (B) Photographic images of lungs from eCPMV-and PBS-treated B16F10 tumor-bearing mice on day 21 post-tumor challenge. (C and D) B16F10 lung metastatic-like tumor foci were quantified both by number in (C) or by qRT-PCR assay for melanocyte-specific Tyrp1 mRNA expression in (D).

FIGS. 4A-4C provide graphs showing eCPMV treatment efficacy in B16F10 lung model is immune-mediated. (A) eCPMV inhalation did not significantly affect tumor progression when mice lack Il-12. (B) Treatment efficacy was also abrogated in the absence of Ifn-γ. (C) NOD/scid/Il2R-γ^{-/-} mice lacking T, B, and NK cells also failed to respond to eCPMV inhalation therapy.

FIGS. 5A-5D provide graphs and images showing eCPMV immunotherapy is successful in metastatic breast, flank melanoma, colon, and ovarian carcinoma models. (A) Mice challenged with 4T1 breast tumors and intratracheally injected with PBS rapidly developed (IVIS images) and succumbed (Kaplan-Meier) to metastatic lung tumors beginning on day 24, whereas tumor development was delayed and survival significantly extended in mice receiving intratracheal injection of eCPMV. (B) Mice bearing intradermal flank B16F10 tumors directly injected with eCPMV (arrows indicate treatment days) showed noticeably delayed tumor progression relative to PBS-injected controls and, in half of eCPMV-treated mice, the tumor was eliminated altogether. (C) Mice bearing intradermal flank CT26 colon tumors also responded to direct injection of eCPMV (arrows indicate treatment days) with significantly delayed growth when compared to PBS-injected controls. (D) eCPMV also proved successful as a therapy for ID8-Defb29/Vegf-A ovarian cancer-challenged mice, significantly improving survival when injected IP relative to PBS-injected controls.

FIGS. 6A and 6B provide a time chart (A) and a graph (B) showing eCPMV treatment of dermal B16F10 induces systemic anti-tumor immunity.

FIGS. 7A-D are a schematic and transmission electron micrographs of (A) PVX and (B) CPMV. C+D) Tumor treatment study. Tumors were induced with an intradermal injection of 125,000 cells/mouse. Mice (n=3) were treated with 100 μg of PVX or CPMV (or PBS control) once weekly, starting 8 days post-induction. Arrows indicate injection days; mice were sacrificed when tumor volumes reached 1000 mm3. (C) Tumor growth curves shown as relative tumor volume. (D) Survival rates of treated mice.

FIGS. 8A-E illustrate synthesis and characterization of PVX−DOX. A) Scheme of DOX loading onto PVX. B) Agarose gel electrophoresis of PVX, PVX−DOX, and free DOX under UV light (top) and after Coomassie Blue staining (bottom). C) TEM images of negatively stained PVX−DOX. D) UV/visible spectrum of PVX−DOX. E) Efficacy of PVX−DOX vs. DOX in B16F10 cells after 24 hours exposure (MTT assay).

FIGS. 9A-C illustrate chemo-immunotherapy treatment of B16F10 tumors. Groups (n=6) were treated with PBS, PVX, DOX, PVX−DOX, or PVX+DOX. PVX was administered at a dose of 5 mg kg⁻¹, DOX was administered at a dose of 0.065 mg kg⁻¹. Injections were repeated every other day until tumors reached >1000 mm3. A) Tumor growth curves shown as relative tumor volume. Statistical significance was detected comparing PVX vs. PVX+DOX. B) Survival rates of treated mice. C) Immunofluorescence imaging of three representative PVX−DOX tumor sections after weekly dosing of PVX−DOX (animals received 2 doses of PVX and were collected when tumors reached >1000 mm³. Tumors treated with PVX−DOX (rows 1-3) were sectioned and stained with DAPI (blue), F4/80 (red), and PVX (green). Scale bar=100 μm.

FIG. 10 illustrates Luminex Multiplex Cytokine/Chemokine profiles. Groups (n=4) were treated with PBS, PVX, DOX, PVX−DOX, or PVX+DOX. PVX was administered at a dose of 5 mg kg⁻¹, DOX was administered at a dose of 0.065 mg kg⁻¹. After one injection, tumors were harvested at 24 hours post injection for analysis. Data shown as ratio of cytokine concentration: total protein (or cytokine per total protein in pg/mg). *=<0.05, **=<0.01, ****=<0.001, ****=<0.0001).

FIGS. 11(A-D) are a synthesis and characterization of DOX-loaded PVX (PVX−DOX). (A) Scheme of doxorubicin (DOX) loading onto potato virus X (PVX). (B) TEM image of negatively stained PVX−DOX. (C) Agarose gel electrophoresis of PVX (lane 1), PVX−DOX (lane 2), and DOX (lane 3) under UV light (top) and after Coomassie Blue (CB) staining (bottom). (D) UV/visible spectra of PVX−DOX at 1× and 5× dilution vs. DOX (at the equivalent concentration).

FIGS. 12(A-D) illustrate the efficacy of DOX (black line) vs. PVX−DOX (blue line) in a panel of cell lines including (A) A2780 human ovarian cancer, (B) MDA-MB-231 human breast cancer, and (C) HeLa human cervical cancer as determined by MTT assay. Cells were treated by DOX or PVX−DOX corresponding to 0, 0.01, 0.05, 0.1, 0.5, 1, 5, or 10 μM (A2780 and MDA-MB-231) or 0, 0.1, 0.5, 1, 5, 10, 25, or 50 μM (HeLa) in 24 h. (D) IC50 values of DOX vs. PVX−DOX were determined using GraphPad Prism software (DOX vs. PVX−DOX p<0.05).

FIGS. 13(A-B) illustrate nuclear accumulation of DOX in A2780 cells after incubation with DOX or PVX−DOX over a 48 h time course. DOX accumulation was imaged (A) and quantified (B) using fluorescence microscopy. Scale bar=20 μm. For quantification, at least 3 images were analyzed per sample, with at least 4 cells per image considered. Fluorescence intensity (FI) was quantified by measuring the nuclei using ImageJ software.

FIGS. 14(A-D) illustrate (A) schematic of the PEGylated, DOX-loaded PEG-PVX−DOX. (B-D) Characterization of PEG-PVX−DOX. (B) Agarose gel electrophoresis of PVX (lane 1), PEG-PVX−DOX (lane 2), and DOX (lane 3); the gel was imaged under UV light (top) and after Coomassie Blue (CB) stain under white light (bottom). (C) SDS-PAGE of PVX (lane 1), PVX−DOX (lane 2), and PEG-PVX−DOX (lane 3) under UV light (left) and after staining with CB (right) photographed under white light. M: SeeBlue Plus2 Protein standard. Released DOX, PVX coat proteins (CP) and PEGylated PEG-CP are detected. (D) UV/visible spectra of PEG-PVX−DOX at 1× and 5× dilution vs. DOX (at the equivalent concentration).

FIGS. 15(A-C) illustrate (A) In vitro efficacy of PEG-PVX−DOX vs. DOX determined by MTT assay. DOX or PEG-PVX−DOX corresponding to 0, 0.01, 0.05, 0.1, 0.5, 1, 5, or 10 μM were incubated with MDA-MB-231 cells. IC₅₀ values were determined using GraphPad Prism software (DOX vs. PVX−DOX p<0.05). (B,C) Treatment of MDA-MB-231 tumors in an athymic mouse model using PEG-PVX−DOX vs. DOX and PBS. Treatment started when tumors reached 100-200 mm3; arrows indicate the treatment schedule. Groups of 4 animals were treated with a dosage of 1.0 mg/kg body weight DOX or PEG-PVX−DOX normalized to the DOX dose. (B) Daily monitored tumor volume growth in mice of different groups and (C) tumor volumes normalized to initial tumor volumes at day 0, 10, and 20 post first treatment (middle bars represent median values and the whiskers denote the minimum and maximum values).

FIGS. 16(A-F) illustrate (A) Schematic representation of sulfo-Cy5 conjugation to surface lysine residues on CPMV and PVX through NHS chemistry (CPMV (Protein Data Bank (PDB) entry INY7) and PVX structures/drawing were created with Biorender.com; reaction scheme was created using ChemDraw Ultra 7.0. (B) CPMV native agarose gel stained with GelRed (to visualize nucleic acid; imaged under UV light) and comassie (to visualize protein; imaged under white light); (C) CPMV small (S-CP, 24 kDa) and the large coat proteins (L-CP; 42 kDa) were separated on a denaturing 4-12% Nu-PAGE gel electrophoresis, while PVX coat protein (19 kDa) was separated on a denaturing 12% Nu-PAGE gel electrophoresis; gels were stained with GelCode Blue Safe protein and imaged under white light; prior to protein staining, Cy5 conjugation was confirmed by imaging for both native and denaturing gels under MultiFluor Red channel under 607 nm excitation (fluorescence image) on a FluorChem R imager. (D) UV-Vis spectra for all purified and Cy5-conjugated VNPs; the 260/280 nm ratio and number of dyes/VNPs were calculated and showed in the graph. (E) Size exclusion chromatography (SEC) was used to assess VNPs integrity using a Superose 6 increase column; Cy5 was detected at 647 nm and A260/A280 nm ratio is also reported. (F) Transmission electron microscopy (TEM) images of negatively stained purified and conjugated VNPs. Scale bars are reported in the images and data was analyzed using ImageJ.

FIGS. 17(A-D) illustrate (A) Balb/c mice (n=10/treatment group) were immunized in a prime-boost schedule (100 μg of VNPs/200 μL of PBS), subcutaneously (s.c.) behind the neck. Blood was collected 2 weeks following each injection. A20 lymphoma cells (2×10⁵ cells/mouse) were inoculated intradermally (i.d.) on mice's right flank; when tumors reached ˜30 mm³, CPMV/PVX (100 μg/20 μL of PBS) or PBS (control) were intratumorally injected, three times/weekly. Tumor volume was monitored every other day and survival curves are shown for all treatment groups; data was plotted as mean±SD for a minimum of n=5 per group.; (B) and (C) Survivors (CPMV n=5 and PVX n=3) were rechallenged (A20 lymphoma cells, 2×10⁵ cells/mouse, i.d., opposite flank) and an age-matched (n=8) set of mice was used as tumor growth control; no treatment was performed (only tumor monitoring); (D) After 32 days post-rechallenge; mice were euthanized and spleens were harvested and splenocytes (5×10⁵ cells/well) were stimulated with medium only (NS), CPMV, PVX, A20 cells (5×10⁵ cells/well) or PMA/Iono (positive control). IFN-γ-producing cells were counted splenocyte-forming colonies. Statistical analysis was performed using two-way ANOVA using pairwise multiple comparison followed by Tukey's multiple comparison test was used to compare between groups. (****p<0.0001) and survival curves were compared using log-rank (Mantel-Cox) (***p<0.001) test using GraphPad Prism v10.2.0 software.

FIGS. 18(A-C) illustrate VNPs uptake and immunogenicity of CPMV and PVX. (A) Specific anti-CPMV and anti-PVX antibodies enhanced VNPs uptake over time. Cy5-CPMV or Cy5-PVX uptake by macrophages (Raw 264.7) was analysed by flow cytometry in the presence and absence of immunized mice plasma. VNPs (1 μg) were incubated with mice plasma (1:200) prior incubation with macrophages for 2 h or 8 h. Statistical analysis was performed using 2-way ANOVA-Tukey's multiple comparisons test (**p=0.044, ****p<0.0001) using GraphPad Prism v10.2.0 software. (B) Immunogenicity of CPMV and PVX was assessed by NF-kB/AP-1 activation in RAW Blue™ cells and (C) TLR7/NF-kB Luc reporter-HEK 293 cells. CPMV shows a concentration-dependent NF-kB/AP-1 activation, that is slightly enhanced by the presence of immunized anti-CPMV antibodies. meanwhile PVX seems to not be signaling through this pathway, regardless of anti-PVX antibodies. It is known that CPMV acts as a TLR agonist and activates TLRs 2/4/7; little is known about PVX immunogenicity. Thus, a custom-made TLR7 agonist assay was used to assess if PVX could act as a TLR7 agonist. TLR7/NF-kB Luc reporter-HEK 293 cells were incubated (16 h) with different doses of CPMV or PVX and Ebola GP2 protein (R848) was used as a positive control and luciferase activity was analyzed by Abcomics Inc., CA, USA.

FIGS. 19(A-B) illustrate immunofluorescence imaging of B cell lymphoma clearance by VNPs. Tumors were collected 24 (panel A) and 72 (panel B) hours following a single treatment. A) After 24 hours, the VNP-positive regions of the tumors (top row) show CD19 depletion. Abundant CD19 signal in VNP-negative regions of the tumors (bottom row). B.) After 72 hours, CPMV remains within the tumor, while PVX is cleared (top row). CD19 signal in VNP-negative sections of tumor (bottom row) comparable to PBS control. Scale bar is 50 μm.

FIGS. 20(A-B) illustrate immunofluorescence imaging of neutrophil infiltration in A20 tumors after a single IIT treatment to preimmunized animals. A) After 24 hours, both CPMV and PVX show co-localization with the tumor infiltrating neutrophils. B) After 72 hours, both CPMV and PVX-treated tumors show a clear upregulation of neutrophils even though PVX has been mostly cleared from the tumor. Scale bar is 50 μm.

FIGS. 21(A-B) illustrate immunofluorescence imaging of macrophages infiltration in A20 tumors after a single IIT treatment to preimmunized animals. A) After 24 hours, both CPMV show an enhanced co-localization with the tumor associated macrophages when compared to PVX. B) After 72 hours, PVX has been mostly cleared from the tumor, while CPMV remains within the TME and shows strong interaction with the TAMs. Scale bar is 50 μm.

FIG. 22 Average hydrodynamic diameter (nm) and polydispersity index of CPMV and Cy5-CPMV nanoparticles were measured using dynamic light scattering (DLS).

FIG. 23 illustrates immunogenicity of CPMV and PVX after preimmunization to generate specific anti-VNPs antibodies; mice followed a prime-boost homologous immunization regimen (100 μg particles, biweekly, s.c. behind the neck). Anti-CPMV and anti-PVX antibody titers and end point IgG titers were determined by ELISA. Graphs were made using GraphPad Prism v10.2.0 software.

FIG. 24 illustrates half maximal effective concentration (EC50) of CPMV and PVX agonist activity on TLR7 using TLR7/NF-kB Luc reporter-HEK293 cell line. CPMV shows a moderate TLR7 agonist (EC50=689 μg/mL) while PVX barely showed any TLR7 agonist effect (EC50>1000 μg/mL).

FIG. 25 illustrates CPMV and PVX were packed with lipofectamine vehicle to enhance internalization of the samples and agonist activity on TLR7 was evaluate using the TLR7/NF-kB Luc reporter cell line system.

### DETAILED DESCRIPTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the application pertains. Commonly understood definitions of molecular biology terms can be found in, for example, Rieger et al., Glossary of Genetics: Classical and Molecular, 5th Edition, Springer-Verlag: New York, 1991, and Lewin, Genes V, Oxford University Press: New York, 1994.

### Definitions

As used in the description of the invention and the appended claims, the singular forms “a,” “an,” and “the” are intended to include the plural forms as well, unless the context clearly indicates otherwise. In addition, the recitations of numerical ranges by endpoints include all numbers subsumed within that range (e.g., 1 to 5 includes 1, 1.5, 2, 2.75, 3, 3.80, 4, 5, etc.).

The terms “comprise,” “comprising,” “include,” “including,” “have,” and “having” are used in the inclusive, open sense, meaning that additional elements may be included. The terms “such as”, “e.g.”, as used herein are non-limiting and are for illustrative purposes only. “Including” and “including but not limited to” are used interchangeably.

The term “or” as used herein should be understood to mean “and/or”, unless the context clearly indicates otherwise.

The terms “cancer” or “tumor” refer to any neoplastic growth in a subject, including an initial tumor and any metastases. The cancer can be of the liquid or solid tumor type. Liquid tumors include tumors of hematological origin, including, e.g., myelomas (e.g., multiple myeloma), leukemias (e.g., Waldenstrom's syndrome, chronic lymphocytic leukemia, other leukemias), and lymphomas (e.g., B-cell lymphomas, non-Hodgkin's lymphoma). Solid tumors can originate in organs and include cancers of the lungs, brain, breasts, prostate, ovaries, colon, kidneys and liver.

The terms “cancer cell” or “tumor cell” can refer to cells that divide at an abnormal (i.e., increased) rate. Cancer cells include, but are not limited to, carcinomas, such as squamous cell carcinoma, non-small cell carcinoma (e.g., non-small cell lung carcinoma), small cell carcinoma (e.g., small cell lung carcinoma), basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; hematologic cancers, such as myelomas, leukemias (e.g., acute myelogenous leukemia, chronic lymphocytic leukemia, granulocytic leukemia, monocytic leukemia, lymphocytic leukemia), lymphomas (e.g., follicular lymphoma, mantle cell lymphoma, diffuse large B-cell lymphoma, malignant lymphoma, plasmocytoma, reticulum cell sarcoma, or Hodgkin's disease), and tumors of the nervous system including glioma, glioblastoma multiform, meningoma, medulloblastoma, schwannoma and epidymoma.

The term “nanoparticle” refers to any particle having a diameter of less than 1000 nanometers (nm). In general, the nanoparticles should have dimensions small enough to allow their uptake by eukaryotic cells. Typically the nanoparticles have a longest straight dimension (e.g., diameter) of 200 nm or less. In some embodiments, the nanoparticles have a diameter of 100 nm or less. Smaller nanoparticles, e.g., having diameters of 50 nm or less, e.g., about 1 nm to about 30 nm or about 1 nm to about 5 nm, are used in some embodiments.

The phrases “parenteral administration” and “administered parenterally” are art-recognized terms and include modes of administration other than enteral and topical administration, such as injections, and include, without limitation, intratumoral, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrastemal injection and infusion.

The phrases “systemic administration,” “administered systemically,” “peripheral administration” and “administered peripherally” as used herein mean the administration of a compound, agent or other material other than directly into a specific tissue, organ, or region of the subject being treated (e.g., tumor site), such that it enters the animal's system and, thus, is subject to metabolism and other like processes, for example, subcutaneous administration.

As used herein, the terms “peptide,” “polypeptide” and “protein” are used interchangeably, and refer to a compound comprised of amino acid residues covalently linked by peptide bonds. A protein or peptide must contain at least two amino acids, and no limitation is placed on the maximum number of amino acids that can comprise the sequence of a protein or peptide. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof. A protein may be a receptor or a non-receptor.

A “nucleic acid” refers to a polynucleotide and includes polyribonucleotides and polydeoxyribonucleotides.

“Treating”, as used herein, means ameliorating the effects of, or delaying, halting or reversing the progress of a disease or disorder. The word encompasses reducing the severity of a symptom of a disease or disorder and/or the frequency of a symptom of a disease or disorder.

A “subject”, as used therein, can be a human or non-human animal. Non-human animals include, for example, livestock and pets, such as ovine, bovine, porcine, canine, feline and murine mammals, as well as reptiles, birds and fish. Preferably, the subject is human.

The language “effective amount” or “therapeutically effective amount” refers to a sufficient amount of the composition used in the practice of the invention that is effective to provide effective treatment in a subject, depending on the compound being used. That result can be reduction and/or alleviation of the signs, symptoms, or causes of a disease or disorder, or any other desired alteration of a biological system. An appropriate therapeutic amount in any individual case may be determined by one of ordinary skill in the art using routine experimentation.

A “prophylactic” or “preventive” treatment is a treatment administered to a subject who does not exhibit signs of a disease or disorder, or exhibits only early signs of the disease or disorder, for the purpose of decreasing the risk of developing pathology associated with the disease or disorder.

A “therapeutic” treatment is a treatment administered to a subject who exhibits signs of pathology of a disease or disorder for the purpose of diminishing or eliminating those signs.

“Pharmaceutically acceptable carrier” refers herein to a composition suitable for delivering an active pharmaceutical ingredient, such as the composition of the present invention, to a subject without excessive toxicity or other complications while maintaining the biological activity of the active pharmaceutical ingredient. Protein-stabilizing excipients, such as mannitol, sucrose, polysorbate-80 and phosphate buffers, are typically found in such carriers, although the carriers should not be construed as being limited only to these compounds.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

Embodiments described herein relate to methods of treating cancer in a subject in need thereof by administering in situ to the cancer a therapeutically effective amount of a plant virus or virus-like particle to the subject. The in situ vaccination approach does not rely on the virus or virus-like particles as a vehicle for drug or antigen delivery, but rather on their inherent immunogenicity. In some embodiments, the in situ administration of the virus or virus-like particles can be proximal to a tumor in the subject or directly to the tumor site to provide a high local concentration of the virus or virus-like particles in the tumor microenvironment (TME). The method represents a type of in situ vaccination or intratumoral immunotherapy (IIT), in which application of an immunostimulatory reagent directly to the tumor modifies the tumor microenvironment so that the immune system is able to respond to the tumor.

It was found that plant virus and virus-like particles and their unique therapeutic features, originally observed in lung infectious disease models, could be utilized for a new application: treating cancer, such as lung cancer and melanoma. In situ vaccination or administration of CPMV, TMV or PVX VLPs alone or in combination with a chemotherapeutic in a model of metastatic lung melanoma as well as dermal melanoma and other cancers (breast, colon, ovarian, lymphoma), was found to have striking efficacy in treating the cancer.

The virus or virus-like particles can be nonreplicating and noninfectious in the subject to avoid infection of the subject and can be regarded as safe from a human health and agricultural perspective. In planta production prevents endotoxin contamination that may be a byproduct of other virus or virus-like particle systems derived from E. coli. The virus or virus-like particles are scalable, stable over a range of temperatures (4-60° C.) and solvent: buffer mixtures.

In some embodiments, plant virus particles or virus-like particles (VLPs) in which the viral nucleic acid is not present are administered in situ to cancer of the subject. Virus-like particles lacking their nucleic acid are non-replicating and non-infectious regardless of the subject into which they are introduced. An example of virus-like particles is empty eCPMV, which is RNA-free.

In other embodiments, the plant virus particles include a nucleic acid within the virus particle. If present, the nucleic acid will typically be the nucleic acid encoding the virus. However, in some embodiments the viral nucleic acid may have been replaced with exogenous nucleic acid. In some embodiments, the nucleic acid is RNA, while in other embodiments the nucleic acid is DNA. A virus particle including nucleic acid will still be nonreplicating and noninfectious when it is introduced into a subject which it cannot infect. For example, plant virus particles will typically be nonreplicating and noninfectious when introduced into an animal subject.

In some embodiments, the plant virus is a plant picornavirus. A plant picornavirus is a virus belonging to the family Secoaviridae, which together with mammalian picornaviruses belong to the order of the Picornavirales. Plant picornaviruses are relatively small, non-enveloped, positive-stranded RNA viruses with an icosahedral capsid. Plant picornaviruses have a number of additional properties that distinguish them from other picornaviruses, and are categorized as the subfamily secoviridae. In some embodiments, the virus particles are selected from the Comovirinae virus subfamily. Examples of viruses from the Comovirinae subfamily include Cowpea mosaic virus (CPMV), Broad bean wilt virus 1, and Tobacco ringspot virus. In a further embodiment, the virus particles are from the Genus comovirus. A preferred example of a comovirus is the cowpea mosaic virus particles.

In some embodiments, the plant virus or virus-like particle is a rod-shaped plant virus. A rod-shaped plant virus is a virus that primarily infects plants, is non-enveloped, and is shaped as a rigid helical rod with a helical symmetry. Rod shaped viruses also include a central canal. Rod-shaped plant virus particles are distinguished from filamentous plant virus particles as a result of being inflexible, shorter, and thicker in diameter. For example, Virgaviridae have a length of about 200 to about 400 nm, and a diameter of about 15-25 nm. Virgaviridae have other characteristics, such as having a single-stranded RNA positive sense genome with a 3′-tRNA like structure and no polyA tail, and coat proteins of 19-24 kilodaltons.

In some embodiments, the rod-shaped plant virus or virus-like particle belongs to a specific virus family, genus, or species. For example, in some embodiments, the rod-shaped plant virus belongs to the Virgaviridae family. The Virgaviridae family includes the genus Furovirus, Hordevirus, Pecluvirus, Pomovirus, Tobamovirus, and Tobravirus. In some embodiments, the rod-shaped plant virus belongs to the genus Tobamovirus. In further embodiments, the rod-shaped plant virus belongs to the tobacco mosaic virus (TMV) species. The tobacco mosaic virus has a capsid made from 2130 molecules of coat protein and one molecule of genomic single strand RNA 6400 bases long. The coat protein self-assembles into the rod like helical structure (16.3 proteins per helix turn) around the RNA which forms a hairpin loop structure. The protein monomer consists of 158 amino acids which are assembled into four main alpha-helices, which are joined by a prominent loop proximal to the axis of the virion. Virions are ˜300 nm in length and ˜18 nm in diameter. Negatively stained electron microphotographs show a distinct inner channel of ˜4 nm.

In further embodiments, the rod-shaped plant virus or virus-like particle can be combined with other rod-shaped plant virus particles by means of a thermal transition to form an RNA-free spherical nanoparticle (SNP). A SNP is a spherical arrangement of the coat proteins of a plurality of rod-shaped plant virus particles formed by thermal transition of the rod-shaped virus particles. SNPs can be labeled with suitable chemicals prior or post thermal transition; for example, NHS-based chemistries allow one to conjugate functional molecules to SNPs post thermal transition; the SNPs are stable and remain structurally sound after chemical modification. The SNPs can be formed from rod-shaped plant virus particles (e.g., TMV virus particles) by briefly heating the rod-shaped plant virus particles. For example, the rod-shaped plant virus particles can be induced to undergo a thermal transition into SNPs by heating at about 96°° C. for about 10 to about 20 seconds. The SNPs are formed from the coat proteins of one or more individual rod-shaped plant virus particles. In various embodiments, the SNP can be formed from about 1 to 10 virus particles, from about 10 to about 20 virus particles, from about 20 to about 30 virus particles, from about 30 to about 40 virus particles, or from about 40 to about 50 virus particles. Depending on the nature of the coat proteins, the number of virus particles incorporated, and the virus particle concentration in the solution in which the thermal transition occurs, the spherical nanoparticles can also vary in size. In some embodiments, the SNPs have a size from about 50 nm to about 800 nm. In further embodiments, the SNPs have a size from about 100 to about 300 nm, or from about 150 to about 200 nm.

In other embodiments, the plant virus or plant virus-like particle is an Alphaflexiviridae virus or virus-like particle. The genera comprising the Alphaflexiviridae family include Allexivirus, Botrexvirus, Lolavirus, Mandarivirus, Potexvirus, and Sclerodarnavirus. In further embodiments, the plant virus particle of the vaccine composition is a Potexvirus particle. Examples of Potexvirus include Allium virus X, Alstroemeria virus X, Alternanthera mosaic virus, Asparagus virus 3, Bamboo mosaic virus, Cactus virus X, Cassava common mosaic virus, Cassava virus X, Clover yellow mosaic virus, Commelina virus X, Cymbidium mosaic virus, Daphne virus X, Foxtail mosaic virus, Hosta virus X, Hydrangea ringspot virus, Lagenaria mild mosaic virus, Lettuce virus X, Lily virus X, Malva mosaic virus, Mint virus X, Narcissus mosaic virus, Nerine virus X, Opuntia virus X, Papaya mosaic virus, Pepino mosaic virus, Phaius virus X, Plantago asiatica mosaic virus, Plantago severe mottle virus, Plantain virus X, Potato aucuba mosaic virus, Potato virus X, Schlumbergera virus X, Strawberry mild yellow edge virus, Tamus red mosaic virus, Tulip virus X, White clover mosaic virus, and Zygocactus virus X. In some embodiments, the plant virus like particle is a Potato virus X virus-like particle.

The virus or virus-like particles can be obtained according to various methods known to those skilled in the art. In embodiments where plant virus particles are used, the virus particles can be obtained from the extract of a plant infected by the plant virus. For example, cowpea mosaic virus can be grown in black eyed pea plants, which can be infected within 10 days of sowing seeds. Plants can be infected by, for example, coating the leaves with a liquid containing the virus, and then rubbing the leaves, preferably in the presence of an abrasive powder which wounds the leaf surface to allow penetration of the leaf and infection of the plant. Within a week or two after infection, leaves are harvested and viral nanoparticles are extracted. In the case of cowpea mosaic virus, 100 mg of virus can be obtained from as few as 50 plants. Procedures for obtaining plant picornavirus particles using extraction of an infected plant are known to those skilled in the art. See Wellink J., Meth Mol Biol, 8, 205-209 (1998). Procedures are also available for obtaining virus-like particles. Saunders et al., Virology, 393 (2): 329-37 (2009). The disclosures of both of these references are incorporated herein by reference.

### Cancer Treatment by Virus Particle Administration

This application describes a method of treating cancer in a subject in need thereof by administering in situ a therapeutically effective amount of a plant virus or virus-like particles to the subject. While not intending to be bound by theory, it appears that the plant virus or virus-like particles have an anticancer effect as a result of eliciting an immune response to the cancer. “Cancer” or “malignancy” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features. A “cancer cell” refers to a cell undergoing early, intermediate or advanced stages of multi-step neoplastic progression. The features of early, intermediate and advanced stages of neoplastic progression have been described using microscopy. Cancer cells at each of the three stages of neoplastic progression generally have abnormal karyotypes, including translocations, inversion, deletions, isochromosomes, monosomies, and extra chromosomes. Cancer cells include “hyperplastic cells,” that is, cells in the early stages of malignant progression, “dysplastic cells,” that is, cells in the intermediate stages of neoplastic progression, and “neoplastic cells,” that is, cells in the advanced stages of neoplastic progression.

The cancers treated by a method described herein can include the following: leukemias, such as but not limited to, acute leukemia, acute lymphocytic leukemia, acute myelocytic leukemias, such as, myeloblastic, promyelocytic, myelomonocytic, monocytic, and erythroleukemia leukemias and myelodysplastic syndrome; chronic leukemias, such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease, diffuse large B-cell lymphoma; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, glioblastoma, brain stem glioma, ependymoma, oligodendroglioma, nonglial tumor, acoustic neurinoma, craniopharyngioma, medulloblastoma, meningioma, pincocytoma, pincoblastoma, primary brain lymphoma; breast cancer including but not limited to ductal carcinoma, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytoma and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, fallopian tube cancer, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcinomas such as but not limited to papillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, prostatic intraepithelial neoplasia, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypemephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, sec Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

The inherent immunogenicity resulting from an in situ vaccination approach described herein appears to be uniquely potent when the particles are inhaled or when administered through intratumoral administration into solid tumors or as IP administration when treating disseminated, metastatic cancer. For treatment of lung tumors, the particles can be intratracheally injected into a subject with established lung tumors and this immunostimulatory treatment results in the rejection of those tumors and systemic immunity that prevents growth of distal tumors. The virus-like particles described herein (e.g., CPMV or PVX) alone are able to stimulate systemic anti-tumor immunity. The virus can potentially render the lung microenvironment inhospitable to tumor cell seeding or continued growth.

Primary lung cancer is the second most common cancer in the United States, behind only breast cancer. Additionally, most other majors cancers frequently metastasize to the lung, including breast, bladder, colon, kidney, melanoma, and prostate. Therefore, in some embodiments, the virus particles are used to treat cancer selected from the group consisting of but not limited to melanoma, breast cancer, bladder cancer, kidney cancer, colon cancer, lung cancer, prostate cancer and ovarian cancer. In some embodiments, the virus particles are used to treat lung cancer. Inhalation is a preferred method of administering the virus or virus-like particles when treating lung cancer. However, inhaled virus particles are able to treat cancer beyond the lung as a result of their ability to stimulate a systemic immune response. For example, in some embodiments, the virus particles are used to treat metastatic cancer which has spread to one or more sites beyond the initial point where cancer has occurred. In other embodiments, the virus or virus-like particles can be administered proximal to tumors in other tissues.

In some embodiments, the method can further include the step of administering a therapeutically effective amount of a cancer therapeutic or cancer therapy to the subject. A “cancer therapeutic” or “cancer therapy”, as used herein, can include any agent or treatment regimen that is capable of negatively affecting cancer in an animal, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of an animal with cancer. Cancer therapeutics can include one or more therapies such as, but not limited to, chemotherapies, radiation therapies, hormonal therapies, and/or biological therapies/immunotherapies. A reduction, for example, in cancer volume, growth, migration, and/or dispersal in a subject may be indicative of the efficacy of a given therapy.

In some embodiments, the method can further include the step of administering a therapeutically effective amount of an anticancer therapeutic agent to the subject. The anticancer therapeutic agents can be in the form of biologically active ligands, small molecules, peptides, polypeptides, proteins, DNA fragments, DNA plasmids, interfering RNA molecules, such as siRNAs, oligonucleotides, and DNA encoding for shRNA. In some embodiments, cytotoxic compounds are included in an anticancer agent described herein. Cytotoxic compounds include small-molecule drugs, such as doxorubicin, mitoxantrone, methotrexate, and pyrimidine and purine analogs, referred to herein as antitumor agents.

The anticancer therapeutic agent can include an anticancer or an antiproliferative agent that exerts an antineoplastic, chemotherapeutic, antiviral, antimitotic, antitumorgenic, and/or immunotherapeutic effects, e.g., prevent the development, maturation, or spread of neoplastic cells, directly on the tumor cell, e.g., by cytostatic or cytocidal effects, and not indirectly through mechanisms such as biological response modification. There are large numbers of anti-proliferative agent agents available in commercial use, in clinical evaluation and in pre-clinical development. For convenience of discussion, anti-proliferative agents are classified into the following classes, subtypes and species: ACE inhibitors, alkylating agents, angiogenesis inhibitors, angiostatin, anthracyclines/DNA intercalators, anti-cancer antibiotics or antibiotic-type agents, antimetabolites, antimetastatic compounds, asparaginases, bisphosphonates, cGMP phosphodiesterase inhibitors, calcium carbonate, cyclooxygenase-2 inhibitors, DHA derivatives, DNA topoisomerase, endostatin, cpipodophylotoxins, genistein, hormonal anticancer agents, hydrophilic bile acids (URSO), immunomodulators or immunological agents, integrin antagonists, interferon antagonists or agents, MMP inhibitors, miscellaneous antineoplastic agents, monoclonal antibodies, nitrosoureas, NSAIDs, ornithine decarboxylase inhibitors, pBATTs, radio/chemo sensitizers/protectors, retinoids, selective inhibitors of proliferation and migration of endothelial cells, selenium, stromelysin inhibitors, taxanes, vaccines, and vinca alkaloids.

The major categories that some anti-proliferative agents fall into include antimetabolite agents, alkylating agents, antibiotic-type agents, hormonal anticancer agents, immunological agents, interferon-type agents, and a category of miscellaneous antincoplastic agents. Some anti-proliferative agents operate through multiple or unknown mechanisms and can thus be classified into more than one category.

Examples of anticancer therapeutic agents that can be administered in combination with a plant virus or virus-like particle described herein include Taxol, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; temozolomide, teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anticancer therapeutic agents include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriccin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; silicon phthalocyanine (PC4) sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosamOinoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Other anticancer agents can include the following marketed drugs and drugs in development: Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Arnad), AM-138 (Armad/Kyowa Hakko), IDN-5005 (Indena), Cryptophycin 52 (also known as LY-355703), AC-7739 (Ajinomoto, also known as AVE-8063A and CS-39.HCl), AC-7700 (Ajinomoto, also known as AVE-8062, AVE-8062A, CS-39-L-Ser.HCl, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin Al (also known as BTO-956 and DIME), DDE-313 (Parker Hughes Institute), Fijianolide B, Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, Inanocine (also known as NSC-698666), 3-IAABE (Cytoskeleton/Mt. Sinai School of Medicine), A-204197 (Abbott), T-607 (Tularik, also known as T-900607), RPR-115781 (Aventis), Eleutherobins (such as Desmethyleleutherobin, Desactyleleutherobin, Isoeleutherobin A, and Z-Eleutherobin), Caribacoside, Caribacolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NC1), Resverastatin phosphate sodium, BPR-OY-007 (National Health Research Institutes), and SSR-250411 (Sanofi).

Still other anticancer therapeutic agents include alkylating agents, such as nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.), antimetabolites, such as folic acid analog (e.g., methotrexate), or pyrimidine analogs (e.g., fluorouracil, floxouridine, Cytarabine), purine analogs (e.g., mercaptopurine, thioguanine, pentostatin, vinca alkaloids (e.g., vinblastin, vincristine), epipodophyllotoxins (e.g., etoposide, teniposide), platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, amino glutethimide).

In particular embodiments, anticancer agents include angiogenesis inhibitors such as angiostatin K1-3, DL-α-difluoromethyl-ornithine, endostatin, fumagillin, genistein, minocycline, staurosporine, and (±)-thalidomide; DNA intercalating or cross-linking agents such as bleomycin, carboplatin, carmustine, chlorambucil, cyclophosphamide, cisplatin, melphalan, mitoxantrone, and oxaliplatin; DNA synthesis inhibitors such as methotrexate, 3-Amino-1,2,4-benzotriazine 1,4-dioxide, aminopterin, cytosine β-D-arabinofuranoside, 5-Fluoro-5′-deoxyuridine, 5-Fluorouracil, gaciclovir, hydroxyurea, and mitomycin C; DNA-RNA transcription regulators such as actinomycin D, daunorubicin, doxorubicin, homoharringtonine, and idarubicin; enzyme inhibitors such as S(+)-camptothecin, curcumin, (−)-deguelin, 5,6-dichlorobenz-imidazole 1-β-D-ribofuranoside, etoposine, formestane, fostriecin, hispidin, cyclocreatine, mevinolin, trichostatin A, tyrophostin AG 34, and tyrophostin AG 879, Gene Regulating agents such as 5-aza-2′-deoxycitidine, 5-azacytidine, cholecalciferol, 4-hydroxytamoxifen, melatonin, mifepristone, raloxifene, all trans-retinal, all trans retinoic acid, 9-cis-retinoic acid, retinol, tamoxifen, and troglitazone; Microtubule Inhibitors such as colchicine, dolostatin 15, nocodazole, paclitaxel, podophyllotoxin, rhizoxin, vinblastine, vincristine, vindesine, and vinorelbine; and various other antitumor agents such as 17-(allylamino)-17-demethoxygeldanamycin, 4-Amino-1,8-naphthalimide, apigenin, brefeldin A, cimetidine, dichloromethylene-diphosphonic acid, leuprolide, luteinizing-hormone-releasing hormone, pifithrin, rapamycin, thapsigargin, and bikunin, and derivatives (as defined for imaging agents) thereof.

In some embodiments, the method can further include the step of ablating the cancer. Ablating the cancer can be accomplished using a method selected from the group consisting of cryoablation, thermal ablation, radiotherapy, chemotherapy, radiofrequency ablation, electroporation, alcohol ablation, high intensity focused ultrasound, photodynamic therapy, administration of monoclonal antibodies, immunotherapy, and administration of immunotoxins.

In some embodiments, the step ablating the cancer includes immunotherapy of the cancer. Cancer immunotherapy is based on therapeutic interventions that aim to utilize the immune system to combat malignant diseases. It can be divided into unspecific approaches and specific approaches. Unspecific cancer immunotherapy aims at activating parts of the immune system generally, such as treatment with specific cytokines known to be effective in cancer immunotherapy (e.g., IL-2, interferon's, cytokine inducers).

In contrast, specific cancer immunotherapy is based on certain antigens that are preferentially or solely expressed on cancer cells or predominantly expressed by other cells in the context of malignant disease (usually in vicinity of the tumor site). Specific cancer immunotherapy can be grouped into passive and active approaches.

In passive specific cancer immunotherapy substances with specificity for certain structures related to cancer that are derived from components of the immune system are administered to the patient. The most prominent and successful approaches are treatments with humanized or mouse/human chimeric monoclonal antibodies against defined cancer associated structures (such as Trastuzumab, Rituximab, Cetuximab, Bevacizumab, Alemtuzumab). The pharmacologically active substance exerts is activity as long as a sufficient concentration is present in the body of the patient, therefore administrations have to be repeated based on pharmacokinetic and pharmacodynamic considerations.

On the other hand, active specific cancer immunotherapy aims at antigen-specific stimulation of the patient's immune system to recognize and destroy cancer cells. Active specific cancer immunotherapy therefore, in general, is a therapeutic vaccination approach. There are many types of cancer vaccine approaches being pursued, such as vaccination with autologous or allogeneic whole tumor cells (in most cases genetically modified for better immune recognition), tumor cell lysates, whole tumor associated antigens (produced by means of genetic engineering or by chemical synthesis), peptides derived from protein antigens, DNA vaccines encoding for tumor associated antigens, surrogates of tumor antigens such as anti-idiotypic antibodies used as vaccine antigens, and the like. These manifold approaches are usually administered together with appropriate vaccine adjuvants and other immunomodulators in order to elicit a quantitatively and qualitatively sufficient immune response (many novel vaccine adjuvant approaches are being pursued in parallel with the development of cancer vaccines). Another set of cancer vaccine approaches relies on manipulating dendritic cells (DC) as the most important antigen presenting cell of the immune system. For example, loading with tumor antigens or tumor cell lysates, transfection with genes encoding for tumor antigens and in-vivo targeting are suitable immunotherapies that can be used together with the virus or virus-like particles of the invention for cancer treatment.

In some embodiments, the step of ablating the cancer includes administering a therapeutically effective amount of radiotherapy (RT) to the subject. It has been found that the combination treatment of radiotherapy and CPMV in situ vaccine resulted in significantly reduced tumor growth compared to RT or CPMV treatment alone. Without being bound by theory, it is believed that RT can prime the tumor by debulking the tumor to provide a burst of tumor antigens in the context of immunogenic cell death that fosters specific immune recognition and response to those antigen; in turn, plant virus nanoparticle-mediated immune stimulation can further augment antitumor immunity to protect from outgrowth of metastases and recurrence of the disease. Thus, in some embodiments, RT is administered prior to in situ administration of the plant virus nanoparticle. In some embodiments, administering in situ to the cancer, (e.g., a tumor site) a therapeutically effective amount of a plant virus or virus-like particle to the subject in combination with administering radiotherapy to the subject can result in an increase in tumor infiltrating lymphocytes (TILs), such as tumor infiltrating neutrophils (TINs) at the tumor site of the subject.

Radiotherapy uses high-energy rays to treat disease, usually x-rays and similar rays (such as electrons). Radiotherapy administered to a subject can include both external and internal. External radiotherapy (or external beam radiation) aims high-energy x-rays at the tumor site including in some cases the peri-tumor margin. External radiotherapy typically includes the use of a linear accelerator (e.g., a Varian 2100C linear accelerator). External radiation therapy can include three-dimensional conformal radiation therapy (3D-CRT), image guided radiation therapy (IGRT), intensity modulated radiation therapy (IMRT), helical-tomotherapy, photon beam radiation therapy, proton beam radiation therapy, stereotactic radiosurgery and/or sterotactic body radiation therapy (SBRT).

Internal radiotherapy (brachytherapy) involves having radioactive material placed inside the body and allows a higher dose of radiation in a smaller area than might be possible with external radiation treatment. It uses a radiation source that's usually sealed in an implant. Exemplary implants include pellets, seeds, ribbons, wires, needles, capsules, balloons, or tubes. Implants are placed in your body, very close to or inside the tumor. Internal radiotherapy can include intracavitary or interstitial radiation. During intracavitary radiation, the radioactive source is placed in a body cavity (space), such as the uterus. With interstitial radiation, the implants are placed in or near the tumor, but not in a body cavity.

In some embodiments, a checkpoint inhibitor can be further administered to eradicate suppressive regulatory T cells prior to RT and initiating an effector immune response with the in situ administration of the plant virus nanoparticle. Exemplary checkpoint inhibitors can include CTLA4 and PD-1/PDL-1 inhibitors. The cytotoxic T-lymphocyte-associated antigen 4 (CTLA-4) and programmed death 1 (PD-1) immune checkpoints are negative regulators of T-cell immune function and inhibition of these targets, results in increased activation of the immune system. Therefore, in some embodiments, a checkpoint inhibitor administered to a subject can include a CTLA-4 and/or PD-1 inhibitor. For example, Ipilimumab, an inhibitor of CTLA-4, is approved for the treatment of advanced or unresectable melanoma. Nivolumab and pembrolizumab, both PD-1 inhibitors, are approved to treat patients with advanced or metastatic melanoma and patients with metastatic, refractory non-small cell lung cancer. In addition the combination of ipilimumab and nivolumab has been approved in patients with BRAF WT metastatic or unresectable melanoma.

It has been shown that moderate magnetic nanoparticle hyperthermia (mNPH) treatment administered to a tumor can generate immune-based systemic resistance to tumor rechallenge. Therefore, in some embodiments, a therapeutically effective amount of a moderate magnetic nanoparticle hyperthermia (mNPH) treatment can be administered to the subject in combination with a plant virus-like particle and/or radiotherapy, wherein the mNPH is activated with an alternating magnetic field (AMF) to produce moderate heat. Without being bound by theory, it is believed that plant virus-like particle immune adjuvants, such as a plant virus nanoparticle and/or a mNPH, will combine with RT-induced generation of immunogenic cell death (ICD) to expand the tumor specific effector T cell population causing longer local and distant tumor remission.

A mNPH treatment can include the use of a magnetic iron oxide nanoparticle (IONP). Once administered to the subject intratumorally, the mNPH can, in some embodiments, be activated with an alternating magnetic field (AMF) to produce moderate heat (e.g., 43°/60 min) at the tumor site. In some embodiments the RT is hypofractionated RT (HFRT) that delivers larger but fewer doses/fractions than typical RT therapies. In an exemplary embodiment, a therapeutically effective amount of RT can be administered to a subject in combination with a combination of a plant virus nanoparticle and/or a mNPH for the treatment of oral melanoma.

### Cargo Molecules

In some embodiments, the virus or virus-like particles are loaded with or bonded to a cargo molecule. A variety of different types of cargo molecules can be loaded into or bonded to the virus or virus-like particles. Cargo molecules that are loaded into the virus or virus-like particles must be sufficiently small to fit within the virus capsid (i.e., have a size of 10 nm or less for a typical icosahedral capsid). Preferred cargo molecules for the present invention include antitumor agents. Alternately, rather than being loaded into the virus or virus-like particles, the cargo molecule can be bonded or conjugated to the plant virus or virus-like particles. The term “conjugating” when made in reference to a cargo molecule, such as an anticancer agent and a plant virus particle as used herein, means covalently linking the cargo molecule to the virus or virus-like particles subject to the single limitation that the nature and size of the agent and the site at which it is covalently linked to the virus or virus-like particles do not interfere with the biodistribution of the modified virus or virus-like particles.

In general, cargo molecules can be conjugated to the plant virus or virus-like particles by any suitable technique, with appropriate consideration of the need for pharmacokinetic stability and reduced overall toxicity to the patient. The cargo molecule can be linked to the interior or the exterior of the virus or virus-like particles, while in some embodiments the cargo molecule is linked to both the interior and the exterior of the virus or virus-like particles. The location of the cargo molecule on the interior or exterior can be governed by the amino acids of the viral coat protein.

A cargo molecule can be coupled to a virus or virus-like particle either directly or indirectly (e.g. via a linker group). In some embodiments, the cargo molecule is directly attached to a functional group capable of reacting with the agent. For example, a nucleophilic group, such as an amino or sulfhydryl group, can be capable of reacting with a carbonyl-containing group, such as an anhydride or an acid halide, or with an alkyl group containing a good leaving group (e.g., a halide). Alternatively, a suitable chemical linker group can be used. A linker group can serve to increase the chemical reactivity of a substituent on either the agent or the virus or virus-like particle, and thus increase the coupling efficiency. A preferred group suitable as a site for attaching cargo molecules to the virus or virus-like particle is one or more lysine residues present in the viral coat protein that have a free amino group that can be capable of reacting with a carbonyl-containing group, such as an anhydride or an acid halide, or with an alkyl group containing a good leaving group (e.g., a halide). Viral coat proteins also contain glutamic and aspartic acids. The carboxylate groups of these amino acids also present attractive targets for functionalization using carbodiimide activated linker molecules; cysteines can also be present which facilitate chemical coupling via thiol-selective chemistry (e.g., maleimide-activated compounds). Further, viral coat proteins contain tyrosine, which can be modified using diazonium coupling reactions. In addition, genetic modification can be applied to introduce any desired functional residue, including non-natural amino acids, e.g. alkyne-or azide-functional groups. See Hermanson, G. T. Bioconjugation Techniques. (Academic Press, 2008) and Pokorski, J. K. and N. F. Steinmetz, Mol Pharm 8 (1): 29-43 (2011), the disclosures of which are incorporated herein by reference.

Alternatively, a suitable chemical linker group can be used. A linker group can serve to increase the chemical reactivity of a substituent on either the agent or the virus or virus-like particles, and thus increase the coupling efficiency. Suitable linkage chemistries include maleimidyl linkers, which can be used to link to thiol groups, isothiocyanate and succinimidyl (e.g., N-hydroxysuccinimidyl (NHS)) linkers, which can link to free amine groups, diazonium which can be used to link to phenol, and amines, which can be used to link with free acids such as carboxylate groups using carbodiimide activation. Useful functional groups are present on viral coat proteins based on the particular amino acids present, and additional groups can be designed into recombinant viral coat proteins. It will be evident to those skilled in the art that a variety of bifunctional or polyfunctional reagents, both homo-and hetero-functional (such as those described in the catalog of the Pierce Chemical Co., Rockford, Ill.), can be employed as a linker group. Coupling can be effected, for example, through amino groups, carboxyl groups, sulfhydryl groups or oxidized carbohydrate residues.

Other types of linking chemistries are also available. For example, methods for conjugating polysaccharides to peptides are exemplified by, but not limited to coupling via alpha-or epsilon-amino groups to NaIO₄-activated oligosaccharide (Bocher et al., J. Immunol. Methods 27, 191-202 (1997)), using squaric acid diester (1,2-diethoxycyclobutene-3,4-dione) as a coupling reagent (Tietze et al. Bioconjug Chem. 2:148-153 (1991)), coupling via a peptide linker wherein the polysaccharide has a reducing terminal and is free of carboxyl groups (U.S. Pat. No. 5,342,770), and coupling with a synthetic peptide carrier derived from human heat shock protein hsp65 (U.S. Pat. No. 5,736,146). Further methods for conjugating polysaccharides, proteins, and lipids to plant virus peptides are described by U.S. Pat. No. 7,666,624.

### Dosage and Formulation of Virus or Virus-Like Particles

When used in vivo, the plant virus or virus-like particles can be administered as a pharmaceutical composition, comprising a mixture of the virus or virus-like particles and a pharmaceutically acceptable carrier. The plant virus or virus-like particle may be present in a pharmaceutical composition in an amount from 0.001 to 99.9 wt. %, more preferably from about 0.01 to 99 wt. %, and even more preferably from 0.1 to 95 wt. %.

The virus or virus-like particles, or pharmaceutical compositions comprising these particles, may be administered by any method designed to provide the desired effect. Administration may be local, intratumoral, or in situ to the cancer being treated. For example, the virus or virus-like particles can be administered by direct injection or implantation into or at a tumor site or tumor micro environment. For disperse or metastatic cancers, the virus or virus-like particles can be administered parenterally; for example orally, rectally, intracisternally, intravaginally, intraperitoneally or locally. Parenteral administration methods include intravascular administration (e.g., intravenous bolus injection, intravenous infusion, intra-arterial bolus injection, intra-arterial infusion and catheter instillation into the vasculature), peri-and intra-target tissue injection, subcutaneous injection or deposition including subcutaneous infusion (such as by osmotic pumps), intramuscular injection, intraperitoneal injection, intracranial and intrathecal administration for CNS tumors, and direct application to the target area, for example by a catheter or other placement device.

A preferred method for administering the plant virus or virus-like particle locally to a subject having lung cancer is by inhalation. For example, the virus particles can be administered intratracheally to the lung of the subject. For administration by inhalation, the virus particles are preferably formulated as an aerosol or powder. Various methods for pulmonary delivery of nanoparticles are discussed by Mansour et al., Int J Nanomedicine. 2009; 4:299-319, the disclosure of which is incorporated herein by reference.

The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

Suitable pharmaceutically acceptable carriers may contain inert ingredients which do not unduly inhibit the biological activity of the virus or virus-like particles. The pharmaceutically acceptable carriers should be biocompatible, e.g., non-toxic, non-inflammatory, non-immunogenic and devoid of other undesired reactions upon the administration to a subject. Standard pharmaceutical formulation techniques can be employed, such as those described in Remington's Pharmaceutical Sciences, ibid. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextran) are known in the art (Baker, et al., “Controlled Release of Biological Active Agents”, John Wiley and Sons, 1986).

A pharmaceutically acceptable carrier for a pharmaceutical composition can also include delivery systems known to the art for entraining or encapsulating drugs, such as anticancer drugs. In some embodiments, the disclosed virus or virus-like particles can be employed with such delivery systems including, for example, liposomes, nanoparticles, nanospheres, nanodiscs, dendrimers, and the like. See, for example Farokhzad, O. C., Jon, S., Khademhosseini, A., Tran, T. N., Lavan, D. A., and Langer, R. (2004). “Nanoparticle-aptamer bioconjugates: a new approach for targeting prostate cancer cells.” Cancer Res., 64, 7668-72; Dass, C. R. (2002). “Vehicles for oligonucleotide delivery to tumours.” J. Pharm. Pharmacol., 54, 3-27; Lysik, M. A., and Wu-Pong, S. (2003). “Innovations in oligonucleotide drug delivery.” J. Pharm. Sci., 92, 1559-73; Shoji, Y., and Nakashima, H. (2004). “Current status of delivery systems to improve target efficacy of oligonucleotides.” Curr. Pharm. Des., 10, 785-96; Allen, T. M., and Cullis, P. R. (2004). “Drug delivery systems: entering the mainstream.” Science, 303, 1818-22. The entire teachings of each reference cited in this paragraph are incorporated herein by reference.

Suitable doses can vary widely depending on the therapeutic or imaging agent being used. A typical pharmaceutical composition for administration would be about 0.1 mg to about 10 g per subject per day. However, in other embodiments, doses from about 1 mg to about 1 g, or from about 10 mg to about 1 g can be used. Single or multiple administrations of the compositions may be administered depending on the dosage and frequency as required and tolerated by the subject. In any event, the administration regime should provide a sufficient quantity of the composition of this invention to effectively treat the subject.

The formulations may be conveniently presented in unit dosage form and may be prepared by any of the methods well known in the art of pharmacy. Preferably, such methods include the step of bringing the virus or virus-like particles into association with a pharmaceutically acceptable carrier that constitutes one or more accessory ingredients. In general, the formulations are prepared by uniformly and intimately bringing the active agent into association with a liquid carrier, a finely divided solid carrier, or both, and then, if necessary, shaping the product into the desired formulations. The methods include administering to a subject, preferably a mammal, and more preferably a human, the composition in an amount effective to produce the desired effect.

One skilled in the art can readily determine an effective amount of plant virus, virus-like particles and/or cancer therapeutics to be administered to a given subject, by taking into account factors such as the size and weight of the subject; the extent of disease penetration; the age, health and sex of the subject; the route of administration; and whether the administration is local or systemic. Those skilled in the art may derive appropriate dosages and schedules of administration to suit the specific circumstances and needs of the subject. For example, suitable doses of the virus or virus-like particles to be administered can be estimated from the volume of cancer cells to be killed or volume of tumor to which the virus particles are being administered.

Useful dosages of the active agents can be determined by comparing their in vitro activity and the in vivo activity in animal models. Methods for extrapolation of effective dosages in mice, and other animals, to humans are known in the art. An amount adequate to accomplish therapeutic or prophylactic treatment is defined as a therapeutically-or prophylactically-effective dose. In both prophylactic and therapeutic regimes, agents are usually administered in several dosages until an effect has been achieved.

The methods described herein contemplate single as well as multiple administrations, given either simultaneously or over an extended period of time. A pharmaceutically acceptable composition containing the plant virus, virus-like particle, and/or additional cancer therapeutic can be administered at regular intervals, depending on the nature and extent of the cancer's effects, and on an ongoing basis. Administration at a “regular interval,” as used herein, indicates that the therapeutically effective amount is administered periodically (as distinguished from a one-time dose). In one embodiment, the pharmaceutically acceptable composition containing the plant virus, virus-like particle, and/or an additional cancer therapeutic is administered periodically, e.g., at a regular interval (e.g., bimonthly, monthly, biweekly, weekly, twice weekly, daily, twice a day or three times or more often a day).

The administration interval for a single individual can be fixed, or can be varied over time, depending on the needs of the individual. For example, in times of physical illness or stress, or if disease symptoms worsen, the interval between doses can be decreased.

For example, the administration of a plant virus or virus like particle and/or the additional therapeutic agent can take place at least once on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least once on week 1, 2, 3, 4, 5, 6, 7, 8, 9, 10, 11, 12, 13, 14, 15, 16, 17, 18, 19 or 20, or any combination thereof, using single or divided doses of every 60, 48, 36, 24, 12, 8, 6, 4, or 2 hours, or any combination thereof. Administration can take place at any time of day, for example, in the morning, the afternoon or evening. For instance, the administration can take place in the morning, e.g., between 6:00 a.m. and 12:00 noon; in the afternoon, e.g., after noon and before 6:00 p.m.; or in the evening, e.g., between 6:01 p.m. and midnight.

In some embodiments, the frequency of administration of plant virus or virus-like particles can pose challenging for clinical implementation. Therefore, in some embodiments, the plant virus or virus-like particles administered in situ to a subject can be formulated in a slow release formulation in order to sustain immune stimulation by maintaining a therapeutic concentration of the plant virus or virus-like particles in situ while alleviating the need for frequent administrations. In some embodiments, a slow release formulation can include a polymer-based hydrogel or a dendrimer.

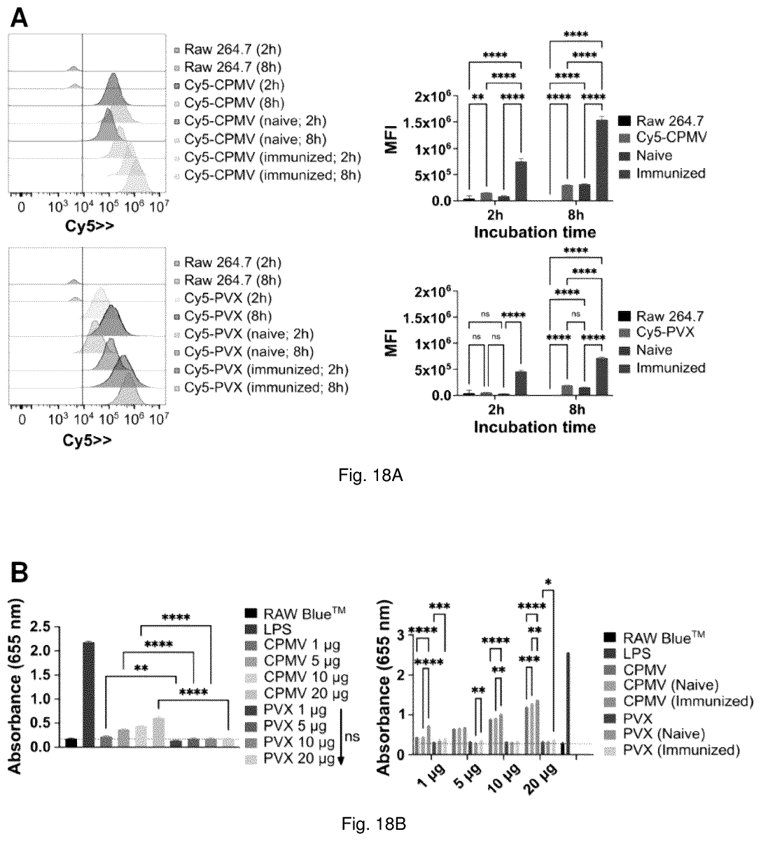

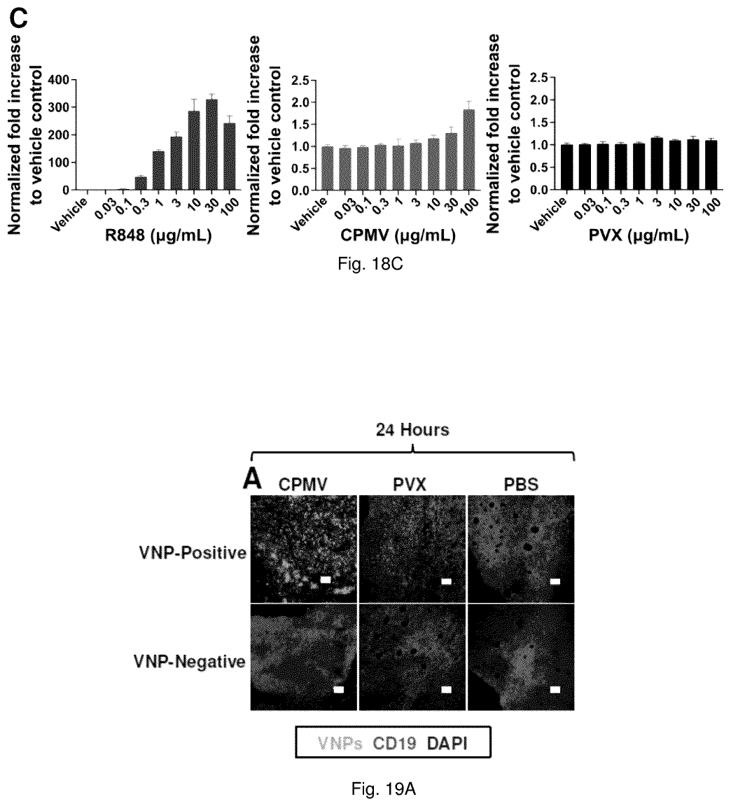

In some embodiments, a slow-release formulation can include a plant virus or virus-like particles dendrimer hybrid aggregate. The dendrimer can include a positively-charged polyamidoamine (PAMAM) dendrimer, such as a medium-sized generation 3 (G3) or generation 4 (G4) PAMAM dendrimer. Depending on the specific application, the plant virus-like particle-dendrimer hybrid aggregates can vary in size and release rate of the plant virus or virus-like particle from the dendrimer when administered to a subject. In some embodiments, the virus or virus-like particle-dendrimer hybrid aggregates are formulated so that at low salt the assembly of the aggregates is triggered and while under physiologic salt concentrations disassembly and plant virus nanoparticle release is induced. In an exemplary embodiment, CPMV and polyamidoamine generation 4 dendrimer form aggregates (CPMV-G4), see FIG. 18A. In particular embodiments, the plant virus-like particle-dendrimer hybrid aggregates, such as CPMV-G4 aggregates, can be administered in situ for the treatment of ovarian cancer or gliomas.

Examples have been included to more clearly describe particular embodiments of the invention. However, there are a wide variety of other embodiments within the scope of the present invention, which should not be limited to the particular examples provided herein.

### EXAMPLES

### Example 1

### In Situ Vaccination With Plant-Derived Virus-Like Nanoparticle Immunotherapy Suppresses Metastatic Cancer

Current therapies are often ineffective for metastatic cancer and emerging immunotherapies, while promising, are early in development. In situ vaccination refers to a process in which immunostimulatory reagents applied directly to the tumor modify the immunosuppressive microenvironment so that the immune system is able to effectively respond against the tumors. The inventors hypothesized that treatment of lung tumor-bearing mice with a virus-like nanoparticle could modulate the lung immune environment and prevent the development of B16F10 metastatic-like lesions. This example shows that inhalation of a self-assembling virus-like particle derived from cowpea mosaic virus (CPMV) suppresses the development of tumors in the lungs of mice after intravenous challenge. The disparity in tumor burden between CPMV-and PBS-treated mice was pronounced, and the effect was immune-mediated as it was not seen in Ifn-γ^{-/-}, Il-12^{-/-}, or NOD/scid/Il2Rγ^{-/-}mice. Efficacy was also lost in the absence of Ly6G⁺ cells. CPMV nanoparticles were rapidly taken up by granulocytic cells in the tumor microenvironment and resulted in their robust activation and cytokine/chemokine production. CPMV nanoparticles are stable, nontoxic, highly scalable, and modifiable with drugs and antigens. These properties, combined with their inherent immunogenicity and significant efficacy against a poorly immunogenic tumor, present CPMV as an attractive novel immunotherapy against cancer metastatic to the lung. Additionally, CPMV exhibited clear treatment efficacy in various other tumor models including dermal melanoma, metastatic breast, colon, and ovarian cancers. This is the first report of a virus-like nanoparticle being utilized as a cancer immunotherapy with proven therapeutic efficacy.

### Materials and Methods

### eCPMV Production and Characterization

eCPMV capsids were produced through agroinfiltration of Nicotiana benthamiana plants with a culture of Agrobacterium tumefaciens LBA4404 transformed with the binary plasmid pEAQexpress-VP60-24K, which contains genes for the coat protein precursor VP60 and its 24K viral proteinase to cleave it into its mature form. 6 days post infiltration, the leaves were harvested and eCPMV extracted using established procedures. The particle concentration was measured using UV/vis spectroscopy (ε₂₈₀ nm=1.28 mg⁻¹ mL cm⁻¹), and particle integrity was determined by transmission electron microscopy and fast protein liquid chromatography.

### Mice

C57BL/6J (01C55) females were purchased from the National Cancer Institute or The Jackson Laboratory. Il-12p35^{-/-} (002692), Ifn-γ^{-/-(}002287), BALB/c (000651), and NOD/scid/Il2Rγ^{-/-}(005557) female mice were purchased from The Jackson Laboratory. All mouse studies were performed in accordance with the Institutional Animal Care and Use Committee of Dartmouth.

### Tumor Models

The B16F10 murine melanoma cell line was obtained from Dr. David Mullins (Geisel School of Medicine at Dartmouth College, Hanover, New Hampshire). 4T1-luciferase murine mammary carcinoma cells were provided by Ashutosh Chilkoti (Duke University, Durham, NC). B16F10, 4T1-luc, and CT26 were cultured in complete media (RPMI supplemented with 10% FBS and penicillin/streptomycin). ID8-Defb29/Vegf-A orthotopic ovarian serous carcinoma cells were cultured in complete media supplemented with sodium-pyruvate as previously described. Lizotte et al., Oncoimmunology, 3: e28926. eCollection 2014. Cells were harvested, washed in phosphate-buffered saline (PBS), and injected in the following manner depending on tumor model: 1.25×10⁵ live cells injected intravenously in 200 μL PBS in the tail vein (B16F10 metastatic lung), 1.25×10⁵ live cells injected intradermally in 30 μL PBS in the right flank (B16F10 flank), 1×10⁵ live cells injected intradermally in 30 μL PBS in the right flank (CT26 flank), 2×10⁶ live cells injected intraperitoneally in 200 μL PBS (ID8-Defb29/Vegf-A peritoneal ovarian). For 4T1-luc tumor challenge, 1×10⁵ live cells were injected in 30 μL of PBS into the left mammary fat pad on day 0 and the tumor was surgically removed on day 16, a day by which it is well-established that the tumor has spontaneously metastasized to the lung. Complete removal of primary 4T1-luc tumors was confirmed by bioluminescent imaging. B16F10 and ID8-Defb29/Vegf-A are syngeneic for the C57BL6J strain, whereas CT26 and 4T1-luc are syngeneic for the BALB/c background.

### eCPMV Treatment Scheduling

WT, Il-12^{-/-}, Ifn-γ^{-/-}, NOD/scid/IL2Ry^{-/-}, and Ly6G-depleted mice challenged intravenously with B16F10 were intubated and intratracheally injected with 100 μg of eCPMV in 50 μL PBS on days 3, 10, and 17 post-tumor challenge. For lung challenge experiments, mice were euthanized on day 21 for quantification of metastatic-like lesions and tyrosinase expression. 4T1-luc-bearing mice were intratracheally injected with 20 μg of eCPMV in 50 μL PBS on day 16 (same day as primary tumor removal) and again on day 23 (day 7 post-tumor removal). B16F10 flank tumors were intratumorally injected with 100 μg eCPMV on day 7 post-tumor challenge once tumors had reached 10 mm² and again on day 14. CT26 flank tumors were intratumorally injected with 100 μg eCPMV on day 8 post-tumor challenge once they had reached 10 mm² and again on day 15. Flank tumor diameters were measured every other day and mice were euthanized when tumor diameters reached 200 mm². ID8-Defb29/Vegf-A mice were injected IP with 100 μg of eCPMV weekly beginning on day 7 post-tumor challenge and euthanized when they reached 35 g due to ascites development.

### Antibodies and Flow Cytometry

Anti-mouse antibodies were specific for CD45 (30-F11), MHC-II (M5/114.15.2), CD86 (GL-1), CD11b (M1/70), F4/80 (BM8), and Ly6G (1A8) from Biolegend and CD16/CD32 (93) from eBioscience. WT and B16F10 lung tumor-bearing mice were intratracheally injected with 100 μg Alexa-488-labeled CPMV particles 24 hr prior to euthanization. Lungs were harvested and dissociated into single cell suspension using the Miltenyi mouse lung dissociation kit (cat #130-095-927). Red blood cells were removed using lysis buffer of 150 mM NH4Cl, 10 mM KHCO3, and 0.5 mM EDTA. Flow cytometry was performed on a MACSQuant analyzer (Miltenyi). Data were analyzed using FlowJo software version 8.7.

### Tyrosinase mRNA Expression Analysis

Whole lungs were dissociated and total RNA was extracted using the RNeasy kit (Qiagen, 74104). cDNA was synthesized using iScript™ cDNA synthesis kit (Bio-Rad, 170-8891). q-PCR was performed on a CFX96™ Real-Time PCR Detection System (Bio-Rad) using iQ™ SYBR® Green Supermix (Bio-Rad, 170-8882) with primers at a concentration of 0.5 μM. mRNA transcript fold-change was calculated using the AACT method with all samples normalized to mouse Gapdh.

### Cytokine Assay

For in vivo cytokine data, total lung homogenate was harvested from B16F10 lung tumor-bearing mice 24 hr post-inhalation of 100 μg eCPMV particles, which was day 8 post-tumor challenge. For in vitro cytokine results, bone marrow-derived dendritic cells (BMDCs) and thioglycollate-stimulated peritoneal macrophages, both derived from C57BL6 mice, were cultured at 1×10⁶ cells/well in 200 μL complete media in 96-well round-bottomed plates with either 20 μg of eCPMV or PBS. Supernatant was harvested after 24 hr incubation. Cytokines were quantified using mouse 32plex Luminex assay (MPXMCYTO70KPMX32, Millipore).

### Cell Depletion

Mice were injected with mAb depleting Ly6G (clone 1A8) that was purchased from Bio-X-Cell (cat #BE0075-1) and administered IP in doses of 500 μg one day prior to eCPMV treatment and then once weekly for the duration of survival experiments. Greater than 95% depletion of target cell populations in the lung was confirmed by flow cytometry.

### IVIS Imaging

Mice were injected IP with 150 mg/kg of firefly D-luciferin in PBS (PerkinElmer cat #122796) and allowed to rest for 10 min. Imagining was conducted using the Xenogen VivoVision IVIS Bioluminescent and Fluorescent Imager platform and analyzed with Living Image 4.3.1 software (PerkinElmer).

### Statistics

Unless noted otherwise, all experiments were repeated at least 2 times with 4-12 biological replicates and results were similar between repeats. Figures denote statistical significance of p<0.05 as *, p<0.01 as **, and p<0.001 as ***. A p-value<0.05 was considered to be statistically significant. Data for bar graphs was calculated using unpaired Student's t-test. Error bars represent standard error of the mean from independent samples assayed within the represented experiments. Flank tumor growth curves were analyzed using two-way ANOVA. Survival experiments utilized the log-rank Mantel-Cox test for survival analysis. Statistical analysis was done with GraphPad Prism 4 software.

### Results

### eCPMV Nanoparticles Are Inherently Immunogenic

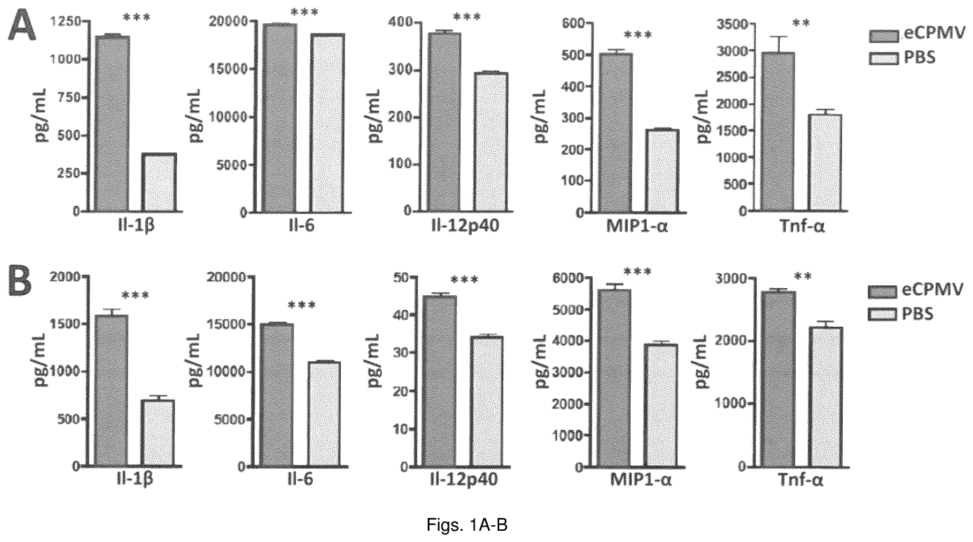

Previously published work with VLPs as treatments for pathogenic infections of the respiratory tract utilized a variety of systems and report varying degrees of immunomodulatory capacity. Additionally, these studies often focused on the immune responses to antigens contained in the VLP and not the inherent immunogenicity of the particles themselves, which has not been definitively shown for VLPs. Bessa et al., Eur J Immunol. 38(1): 114-26 (2008). Moreover, it is not known if some VLPs are more stimulatory than others. For these reasons, and the inventors proposed use of eCPMV (eCPMV refers to “empty” cowpea mosaic virus particle devoid of RNA) as a novel immunotherapy, they first sought to determine its inherent immunogenicity. eCPMV VLPs were added to in vitro cultures of bone marrow-derived dendritic cells (BMDCs) and primary macrophages harvested from C57BL6 mice. Twenty-four hours of culture with eCPMV particles induced both BMDCs (FIG. 1A) and macrophages (FIG. 1B) to secrete higher levels of canonical pro-inflammatory cytokines including Il-β, Il-6, Il-12p40, Ccl3 (MIP1-α), and Tnf-α, leading the inventors to conclude that eCPMV is inherently immunostimulatory.

### eCPMV Inhalation Radically Alters the B16F10 Lung Tumor Microenvironment

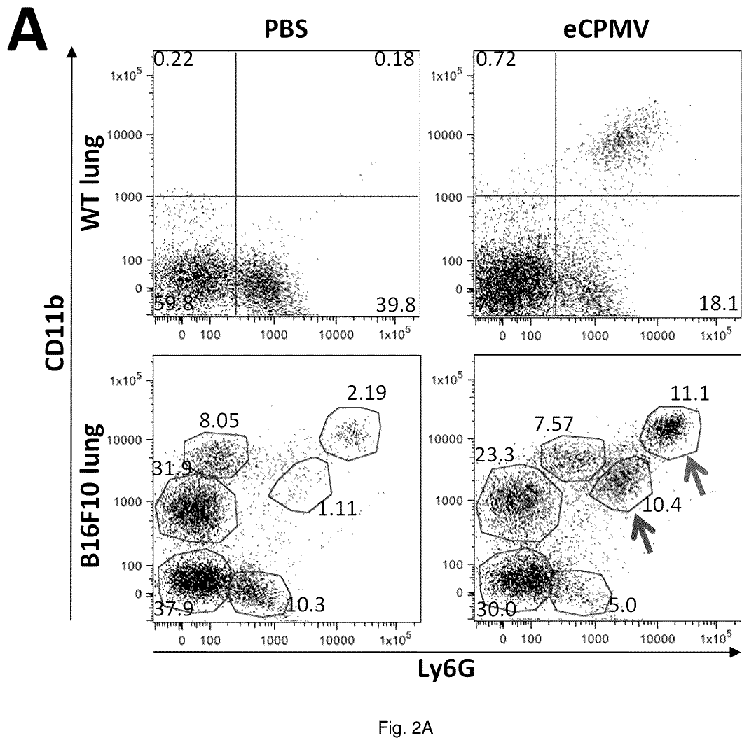

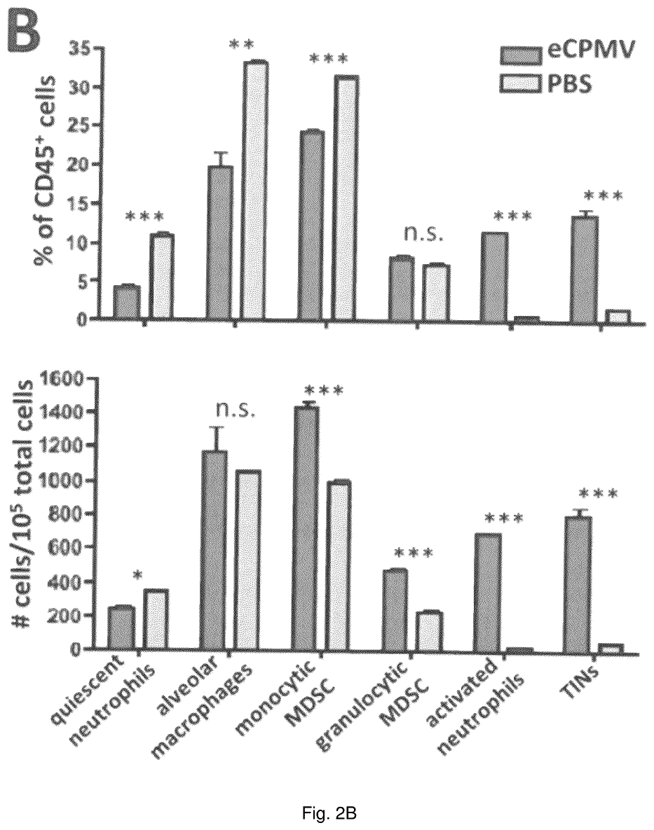

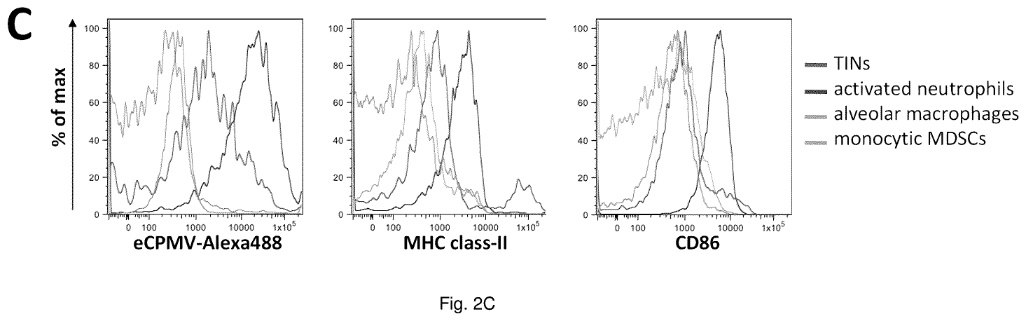

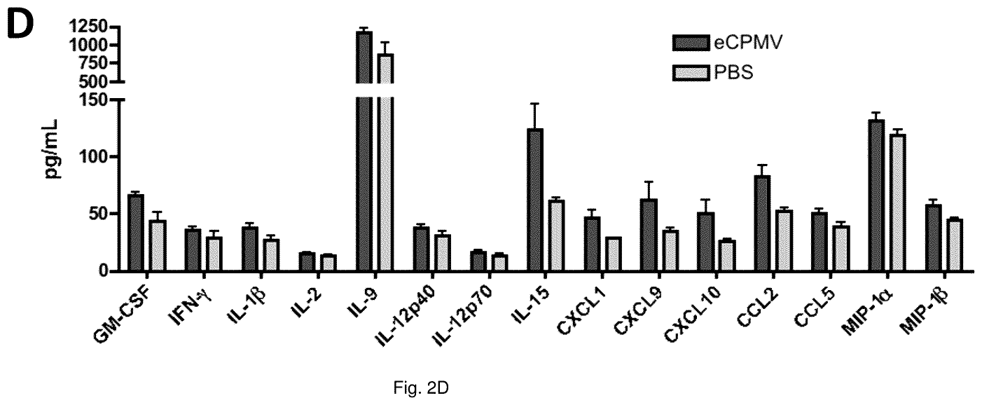

The immunomodulatory effect of eCPMV inhalation on the lung microenvironment was determined next, both in terms of immune cell composition and changes in cytokine and chemokine levels. Exposure of non-tumor-bearing mouse lungs to eCPMV revealed significant activation of Ly6G⁺ neutrophils 24 hours after exposure as assessed by their upregulation of the CD11b activation marker (Costantini et al., Int Immunol., 22 (10):827-38 (2010)) (FIG. 2A top panels) and CD86 co-stimulatory marker. Alexa488-labeling of the particle allowed for cell tracking, which enabled the inventors to confirm that it is this CD11b⁺Ly6G⁺ activated neutrophil subset, specifically, that takes up the eCPMV.

Lungs of mice bearing B16F10 melanoma tumors revealed a more complex immune cell composition. By day 7 the emergence of large populations of immunosuppressive CD11b⁺Ly6G-F4/80^{lo}class-II-SSC^{lo} monocytic myeloid-derived suppressor cells (MDSCs) and CD11b⁺Ly6G⁺F4/80-class-II^{mid}SSC^{hi} granulocytic MDSCs (FIG. 2A bottom panels) could be observed. Gabrilovich et al., Nat Rev Immunol., (4):253-68 (2012). The inventors also observed the presence of a small population of CD11b⁺Ly6G⁺class-II^{mid}CD86^{hi} cells that have been described in the literature as “tumor-infiltrating neutrophils” or “N1 neutrophils” that are known to exert an anti-tumor effect through coordination of adaptive immune responses, production of high levels of pro-inflammatory cytokines, recruitment of T and NK cells, and direct cytotoxicity to tumor cells. Fridlender et al., Cancer Cell. 16(3):183-94 (2009); Mantovani et al., Nat Rev Immunol. (8):519-31 (2011). Inhalation of eCPMV into B16F10-bearing lungs dramatically altered the immune cell composition 24 hours after administration. Significant increases in the tumor-infiltrating neutrophil (TIN) and CD11b⁺Ly6G⁺ activated neutrophils populations, as well as a reduction in CD11b⁻Ly6G⁺ quiescent neutrophils (FIG. 2A bottom panels, see arrows) were also observed. TIN and activated CD11b⁺ neutrophil populations increased dramatically both as a percentage of CD45⁺ cells and also in total number (FIG. 2B). Interestingly, it is these neutrophil subpopulations that took up the vast majority of eCPMV particles, particularly TINs that took up 10-fold more eCPMV than CD11b⁺ activated neutrophils (FIG. 2C). Monocytic MDSC, quiescent neutrophil, and alveolar macrophage populations did not take up eCPMV, and granulocytic MDSCs displayed uneven uptake. The TIN and activated neutrophil populations also expressed MHC class-II, and the TINs, in particular, displayed high levels of co-stimulatory marker CD86, indicating potential antigen presentation and T cell priming capability. Significant changes were not observed in the numbers of monocytic or granulocytic MDSCs.

Activation of neutrophil populations by eCPMV is consistent with data collected from a multiplexed cytokine/chemokine array performed on whole lung homogenate of B16F10 tumor-bearing lungs treated with eCPMV or PBS (FIG. 2D). Specifically, significant increases in neutrophil chemoattractants GM-CSF, Cxcl1, Ccl5, and MIP-1α and significant increases in cytokines and chemokines known to be produced by activated neutrophils such as GM-CSF, Il-1β, Il-9, Cxcl1, Cxcl9, Cxcl10, Ccl2, MIP-1α, and MIP-1β were seen. Interestingly, the inventors did not observe significant increases in levels of Il-6 or Tnf-α, which are classical pro-inflammatory cytokines which may be detrimental in the context of lung immunobiology.

### eCPMV Inhalation Suppresses B16F10 Metastatic-Like Lung Tumor Development

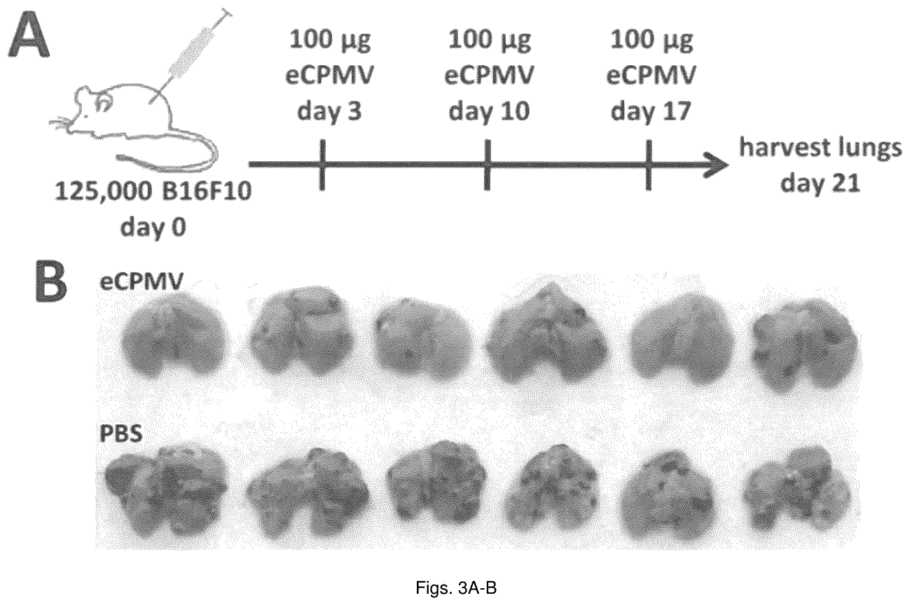

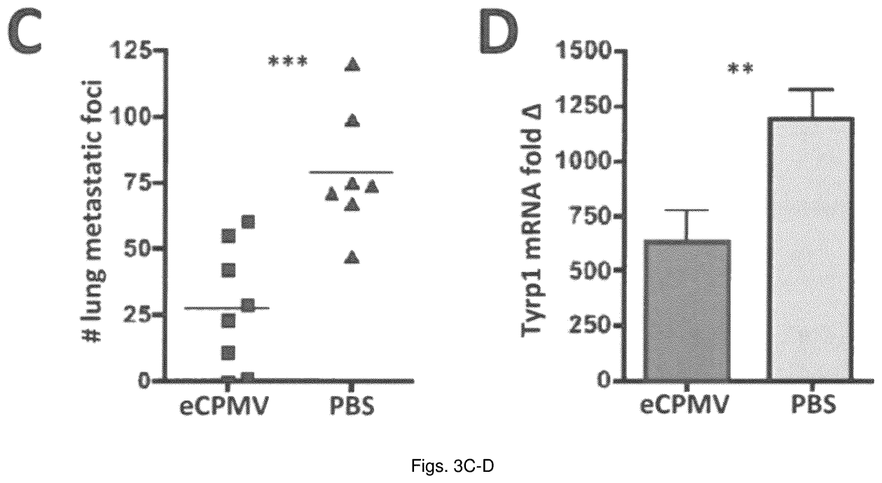

The inventors next investigated whether the inherent immunogenicity of the eCPMV particle in the lung could induce anti-tumor immunity in the B16F10 intravenous model of aggressive metastatic lung cancer. Indeed, weekly intratracheal injection of 100 μg of eCPMV (FIG. 3A) resulted in significantly reduced tumor burden as assessed by both metastatic-like tumor foci number (FIG. 3, B and C) and tyrosinase expression (FIG. 3D). Tyrosinase-related protein 1 (Tyrp1) is a melanocyte-specific gene (Zhu et al., Cancer Res., 73 (7):2104-16 (2013)) whose expression in the lung is restricted to B16F10 tumor cells, which allows for the quantitative measure of tumor development and serves as a control for the varying sizes of metastatic-like foci.

### Anti-Tumor Efficacy of eCPMV Inhalation Is Immune-Mediated

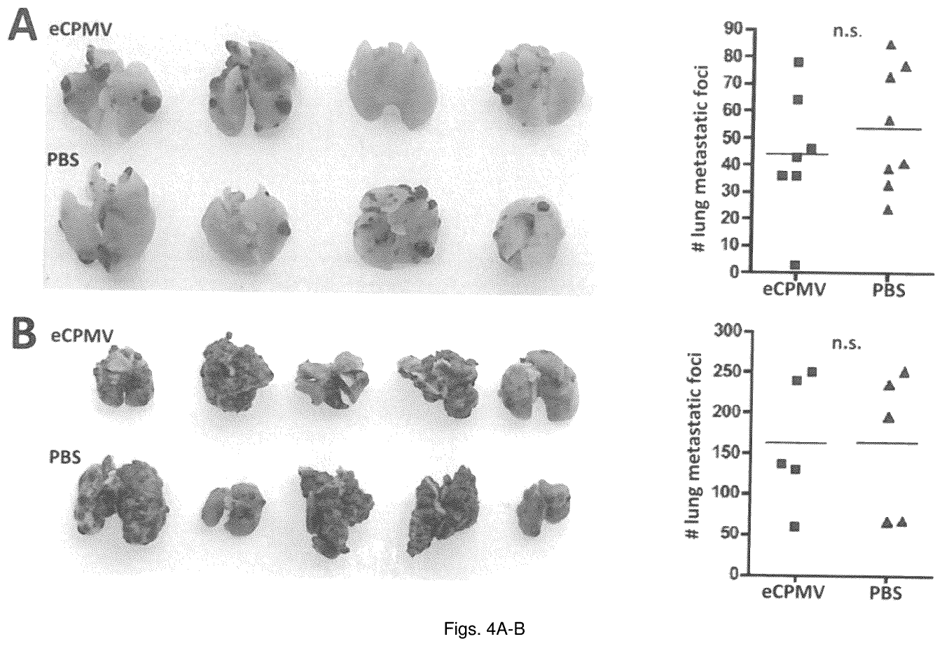

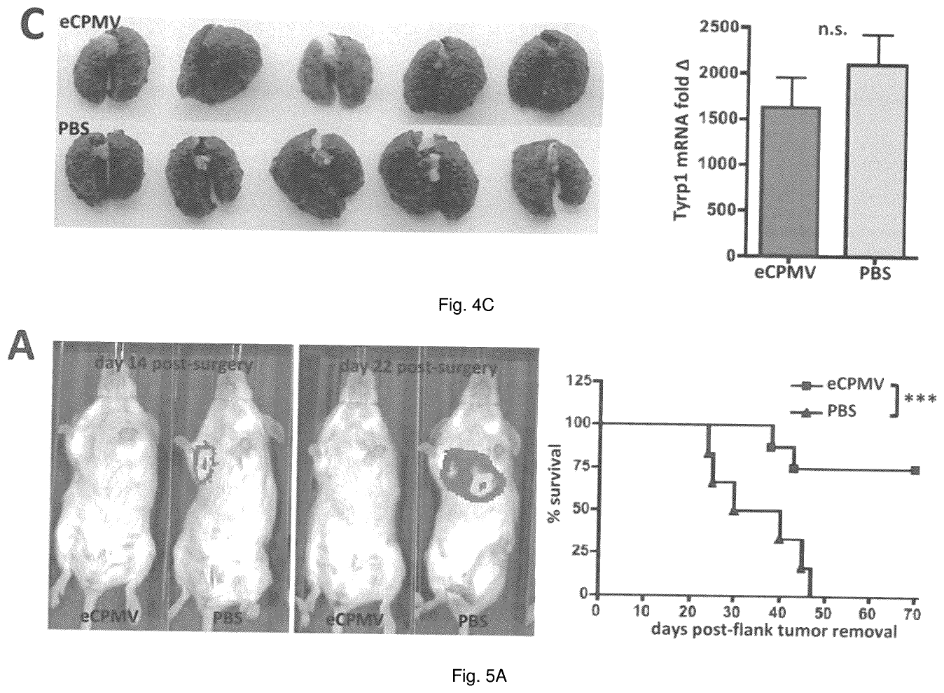

The inventors determined whether the immune system was required for treatment efficacy by repeating their experimental design described in FIG. 3 in the following transgenic mice: Il-12^{-/-}, Ifn-γ^{-/-}, and NOD/scid/IL2Rγ^{-/-}. A significant difference in lung tumor burden between eCPMV-and PBS-treated mice was not observed in the absence of Il-12 (FIG. 4A), Ifn-γ (FIG. 4B), or in NSG mice lacking T, B, and NK cells (FIG. 4C).

eCPMV accumulates in and activates neutrophils in the lungs of B16F10 tumor-bearing mice (FIG. 2, A to C). Additionally, significant increases in neutrophil-associated cytokines and chemokines were detected in the mouse lung following eCPMV inhalation (FIG. 2D). Therefore, neutrophils were depleted using a monoclonal antibody to assess the necessity of neutrophils for treatment efficacy. Depletion of neutrophils from the lungs of tumor-bearing mice abrogated the anti-tumor effect that we observed in WT mice (FIG. 4D). This, combined with the lack of efficacy observed in Il-12^{-/-}, Ifn-γ^{-/-}, and NSG mice leads the inventors to conclude that the anti-tumor effect of eCPMV inhalation on B16F10 development is through immunomodulation of the lung tumor microenvironment and, in particular, requires the presence of the neutrophil compartment.

### eCPMV Anti-Tumor Efficacy Is Not Restricted to the B16F10 Intravenous Lung Model

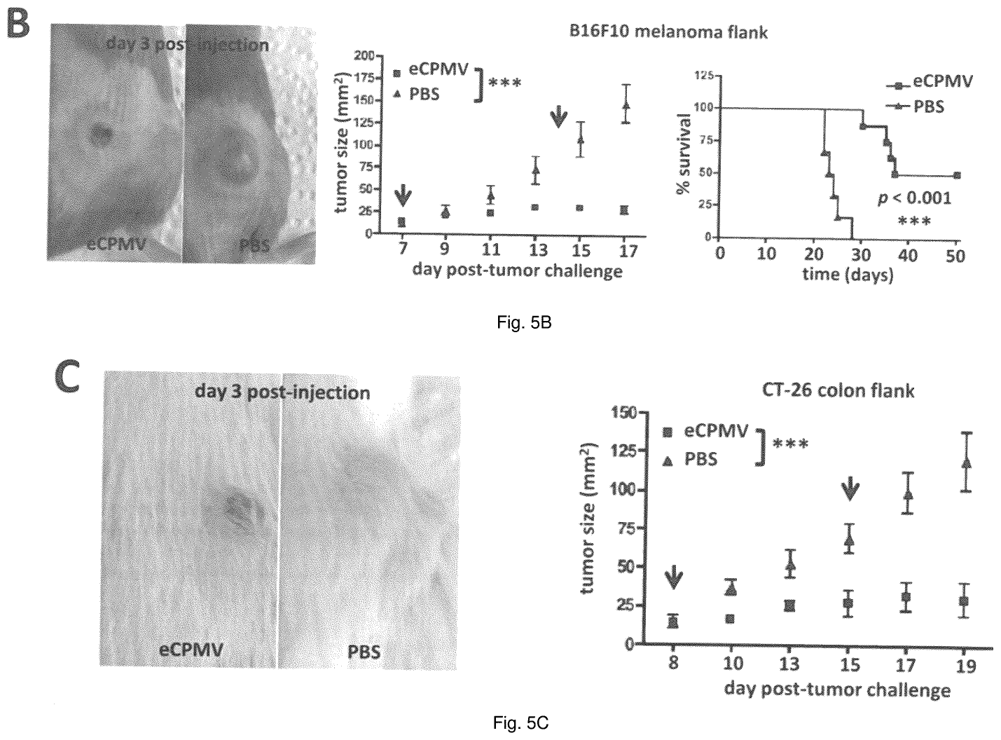

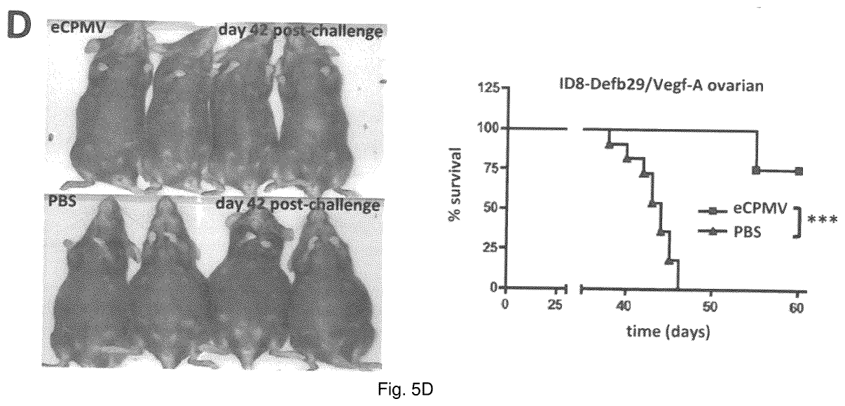

The inventors sought to ascertain whether eCPMV treatment efficacy was restricted to the B16F10 metastatic lung model or if the immunomodulatory anti-tumor effect could transfer to other models. The 4T1 BALB/c syngeneic breast cancer model, which is a transferrable yet truly metastatic model, was first utilized. 4T1 tumors established in the mammary fat pad spontaneously metastasize to the lung by day 16, at which point the primary tumor was surgically removed and eCPMV treatment begun, injecting intratracheally to affect lung tumor development. The 4T1 cells also expressed luciferase, allowing the inventors to track metastatic lung tumor development. Mice treated intratracheally with eCPMV particles had significantly delayed lung tumor onset and significantly extended survival (FIG. 5A). Mice from eCPMV and PBS treatment groups had comparable primary mammary fat pad tumor burden at day of surgical removal. Therefore, differences in tumor development and survival were due to treatment. No mice from either group experienced recurrence of primary mammary fat pad tumors.

To determine whether the eCPMV particle's unambiguous efficacy in B16F10 and 4T1 metastatic lung models was unique to the lung immune environment, its ability to treat dermal tumors was also tested. Both intradermal B16F10 melanoma (FIG. 5B) and CT26 colon (FIG. 5C) tumor growth was significantly delayed following direct injection with eCPMV and, in half of the B16F10 eCPMV-treated mice, resulted in elimination of the tumors altogether after only two treatments (FIG. 5B).

Finally, the therapeutic effect of eCPMV was investigated in a model of disseminated peritoneal serous ovarian carcinoma. Conejo-Garcia et al., Nat Med., (9):950-8 (2004). ID8-Defb29/Vegf-A-challenged mice treated weekly with eCPMV exhibited significantly improved survival relative to PBS-treated controls (FIG. 5D). In fact, mice receiving eCPMV survived longer in comparison than other immunotherapies attempted in this model including a live, attenuated Listeria monocytogenes strain (Lizotte et al., Oncoimmunology, 3:28926. eCollection 2014), avirulent Toxoplasma gondii (Baird et al., Cancer Res., 73 (13): 3842-51 (2013)), combination agonistic CD40 and poly(I:C) (Scarlett et al., Cancer Res., 69 (18): 7329-37 (2009)), or Il-10 blocking antibody (Hart et al., T Cell Biol., 2:29 (2011)). It is important to note that the anti-tumor effects of eCPMV in the tested models are not attributable to direct tumor cell cytotoxicity, as exposure to high concentrations of the particle in vitro had no effect on cancer cell viability or proliferation. The logical conclusion is that the striking anti-tumor effect induced by eCPMV nanoparticle treatment is fully immune-mediated and translatable to a variety of tumor models across diverse anatomical sites.

eCPMV nanoparticles are immunotherapeutic to a surprisingly high degree and clearly modulate the tumor immune environment. BMDCs and macrophages exposed to the particles robustly secreted select pro-inflammatory cytokines (FIGS. 1A and B). eCPMV particles were produced in plants, then plant contaminants were extracted using polyvinyl-polypyrrolidone, eCPMV isolated through PEG precipitation and sucrose gradient ultracentrifugation, and finally the particles concentrated by ultrapelleting. Purity was checked by UV/Vis absorbance, transmission electron microscopy (TEM), fast protein liquid chromatography (FPLC), and SDS gel electrophoresis. Wen et al., Biomacromolecules, 13(12):3990-4001 (2012). No contaminants were detected during these procedures. Particles were then resuspended in PBS. Additionally, eCPMV is completely devoid of RNA that could stimulate TLR3/7. The inventors therefore conclude that the high immunogenicity of eCPMV is due to the size, shape, or inherent immune recognition of the viral coat protein. This is unlike virus-like or protein cage nanoparticles that are manufactured in E. coli or other systems that may contain immunogenic contaminants like endotoxin or viral nucleic acids that are challenging to remove during the purification process.

eCPMV inhalation transforms the lung tumor microenvironment and requires the presence of Ly6G⁺ neutrophils, a population that is minimally associated with response to tumor immunotherapy. Notably, eCPMV particles appear to specifically target Ly6G⁺ neutrophils. eCPMV has been shown to bind surface vimentin on cancer cells (Steinmetz et al., Nanomed. 6(2):351-64 (2011)) and some antigen-presenting cells (Gonzalez et al., PLOS ONE. 4(11):e7981 (2009)), although it is not known if this is the mechanism by which lung-resident neutrophils are internalizing the particle, or if surface expression of vimentin is a feature of murine neutrophils. eCPMV appears to target quiescent neutrophils and convert them to an activated CD11b⁺ phenotype, as well as inducing the recruitment of additional CD11b⁺ activated neutrophils and CD11b⁺class-II⁺CD86^{hi} tumor-infiltrating neutrophils. In fact, these two populations-activated neutrophil and tumor-infiltrating or “N1” neutrophils—are the only innate immune cell populations that significantly change rapidly following eCPMV lung exposure, both dramatically increasing as a percentage of CD45⁺ cells and in total cell number (FIG. 2B). Neutrophils are viewed canonically as sentinels for microbial infection that quickly engulf and kill bacteria before undergoing apoptosis, yet they have an emerging roll in tumor immunology. Although still controversial, it appears that neutrophils possess phenotypic plasticity analogous to the M1/M2 polarization accepted in macrophage literature. Studies have shown infiltration of an immunosuppressive, pro-angiogenic “tumor-associated neutrophil” population in B16F10 metastatic lung, human liver cancer, sarcoma, and lung adenocarcinoma models that is correlated with enhanced tumor progression. Alternatively, depletion of tumor-resident immunosuppressive neutrophils, conversion of them to a pro-inflammatory phenotype, or recruitment of activated neutrophils to infiltrate the tumor microenvironment is associated with therapeutic efficacy. Activated neutrophils can directly kill tumor cells via release of reactive oxygen intermediates (ROI), prime CD4⁺ T cells and polarize them to a Th1 phenotype, cross-prime CD8⁺ T cells, and modulate NK cell survival, proliferation, cytotoxic activity and IFN-γ production. Activated neutrophils can also produce Cxcr3 ligands Cxcl9 and Cxcl10 that can recruit CD4⁺ and CD8⁺ T cells that are correlated with anti-tumor immunotherapeutic efficacy in melanoma models. The data showing increases in immunostimulatory neutrophil populations (FIG. 2B) agrees with the cytokine data (FIG. 2D), in which increases in neutrophil chemoattractants GM-CSF, Cxcl1, Cc15, and MIP-1α and cytokines and chemokines known to be produced by neutrophils were observed, including GM-CSF, Il-1β, Il-9, Cxcl1, Cxc19, Cxcl10, Ccl2, MIP-1α, and MIP-1β. This data in turn agrees with the in vivo tumor progression data showing that neutrophils are required for eCPMV anti-tumor efficacy. Interestingly, although many cytokine and chemokine levels were elevated to a statistically significant degree following eCPMV treatment, changes were modest when compared to the dramatic differences in actual tumor burden (FIG. 3, B to D). Moreover, increases in pro-inflammatory cytokines Tnf-α or Il-6 that are known to cause tissue damage when upregulated in the lung were not observed. It appears, therefore, that eCPMV treatment of lung tumors is effective without eliciting the kind of inflammatory cytokine response that could cause acute lung injury.

eCPMV inhalation exhibited remarkable efficacy as a monotherapy (FIG. 3) that is very clearly immune-mediated (FIG. 4). This is novel because the eCPMV particle does not directly kill tumor cells or share any antigenic overlap with B16F10 tumors, but induces an anti-tumor response that requires Th1-associated cytokines Il-12 (FIG. 4A) and Ifn-γ (FIG. 4B), adaptive immunity (FIG. 4C), and neutrophils (FIG. 4D). This suggests that the inherent immunogenicity of eCPMV, when introduced into the lung, disrupts the tolerogenic nature of the tumor microenvironment; in essence, removing the brakes on a pre-existing anti-tumor immune response that is suppressed, or allowing a de novo anti-tumor response to develop.

This work also shows that eCPMV anti-tumor efficacy in the intravenous B16F10 metastatic lung model is not an artifact of the C57BL6 mouse strain or the B16F10 model, as eCPMV therapy works equally impressively in flank B16F10, ovarian carcinoma, and two BALB/c models of metastatic breast and colon cancer (FIG. 5). Constitutive luciferase expression in 4T1 breast carcinoma cells and intradermal challenges of B16F10 and CT26 allowed us to measure tumor progression quantitatively in a manner not feasible in the B16F10 lung model. It is in these models that we observed a potent and immediate anti-tumor effect that significantly delayed tumor progression and, in the cases of the B16F10 and CT26 intradermal tumors, induced rapid involution of established tumors and formation of necrotic centers (FIG. 5, B and C) that remained confined to within the margins of the tumors and did not appear to affect surrounding tissue. Such early responses to eCPMV—day 3 post-intratumoral injection—would indicate that eCPMV particles are inducing innate immune cell-mediated anti-tumor responses. The eCPMV nanoparticle, alone, is immunogenic and highly effective as a monotherapy. However, it can also serve as a nanocarrier for tumor antigens, drugs, or immune adjuvants, opening up the exciting possibility that eCPMV can be modified to deliver a payload that further augments and improves its immunotherapeutic efficacy.

### Example 2

### eCPMV Treatment of Dermal B16F10 Is Inducing Systemic Anti-Tumor Immunity

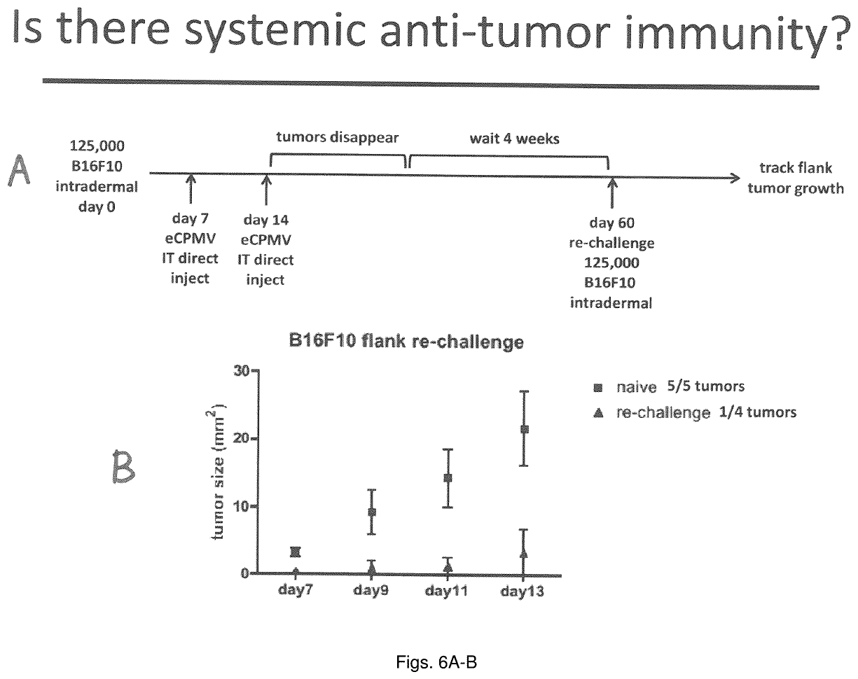

As shown in FIG. 6, the inventors established dermal melanoma tumors and injected the eCPMV particles directly into them (100 μg/injection, arrows indicate injection days). In half of the mice this results in complete disappearance of the tumors. In the cured mice, we then waited 4 weeks and re-challenged with the same tumor cells, but we injected tumor cells on the opposite flank. Most of those mice did not develop secondary tumors. To clarify: primary tumors were directly injected with particles, whereas mice bearing secondary tumors received no treatment. They had to rely on systemic anti-tumor immune memory alone. The eCPMV was applied locally in the first tumors, but induced systemic immunity. “Re-challenge” mice where those that previously had B16F10 dermal tumors that were direct-injected and that shrank and disappeared.

### Example 3

Using a plant virus-based nanotechnology, we demonstrated that virus like particles (VLPs) from the icosahedral virus cowpea mosaic virus (CPMV, 30 nm in diameter) stimulate a potent anti-tumor immune response when applied as an in situ vaccine. Efficacy was demonstrated in mouse models of melanoma, breast cancer, ovarian cancer, and colon cancer. Data indicate that the effect is systemic and durable, resulting in immune-memory and protecting subjects from recurrence. While the underlying mechanism has not been elucidated in depth, initial studies, in which VLPs were inhaled into the lungs of mice bearing B16F10 lung tumors, revealed a sub-population of lung antigen presenting cells (APC) that are MHC class II+CD11b+Ly6G+ neutrophils that ingest VLPs and activate following VLP exposure. Further, the increase in this neutrophil population is accompanied by a decrease of myeloid-derived suppressor cells (MDSCs) that mediate immune-suppression in the tumor microenvironment. Here we set out to investigate the use of plant viruses as in situ vaccines and their combination with chemotherapy regimes.

Recent clinical and preclinical research indicates that the combination of chemo- and immunotherapies can be beneficial because the therapy regimes can synergize to potentiate the therapy and improve patient outcomes. For chemo-immuno combination treatment, the use of the anthracycline doxorubicin (DOX) could be a particularly powerful approach, because DOX itself induces immunogenic cell death that elicits an antitumor immune response. The immune response is induced by calreticulin exposure on the surface of dying cells, which facilitates tumor cell phagocytosis by dendritic cells resulting in tumor antigen presentation. Furthermore, doxorubicin-killed tumor cells recruit intratumoral CD11c+CD11b+Ly6Chi myeloid cells, which present tumor antigens to T lymphocytes; therefore, the combination of doxorubicin with tumor vaccines or immunotherapies can synergize and potentiate the overall efficacy.

### Example 4

In this example, we set out to address the following questions:
i) whether the flexuous particles formed by the potato virus X (PVX) would stimulate an anti-tumor response when used as in situ vaccine?
ii) whether the combination of VLP-based in situ vaccine with DOX chemotherapy would potentiate therapeutic efficacy; specifically we asked whether the formulation as combinatorial nanoparticle where DOX is bound and delivered by PVX (PVX−DOX) or the co-administration of the therapeutic regimens (PVX+DOX) would be the most effective treatment strategy?

All studies were performed using a mouse model of melanoma.

### Methods

### PVX and CPMV Production

PVX was propagated in Nicotiana benthamiana plants and purified as previously reported. CPMV was propagated in Vigna unguiculata plants and purified as previously reported.

### Synthesis of PVX−DOX

PVX (2 mg mL−1 in 0.1 M potassium phosphate buffer (KP), pH 7.0) was incubated with a 5,000 molar excess of doxorubicin (DOX) at a 10% (v/v) final concentration of DMSO for 5 days at room temperature, with agitation. PVX−DOX was purified twice over a 30% (w/v) sucrose cushion using ultracentrifugation (212,000×g for 3 h at 4° C.) and resuspended overnight in 0.1 M KP, pH 7.0. PVX−DOX filaments were analyzed using UV/visible spectroscopy, transmission electron microscopy, and agarose gel electrophoresis.

### UV/Visible Spectroscopy

The number of DOX per PVX filament was determined by UV/visible spectroscopy, using the NanoDrop 2000 spectrophotometer. DOX loading was determined using 20 the Beer-Lambert law and DOX (11,500 M−1 cm−1 at 495 nm) and PVX (2.97 mL mg−1 cm−1 at 260 nm) extinction coefficients.

### Transmission Electron Microscopy (TEM)

TEM imaging was performed after DOX loading to confirm integrity of PVX−DOX filaments. PVX−DOX samples (0.1 mg mL⁻¹, in dH₂O) were placed on carbon-coated copper grids and negatively stained with 0.2% (w/v) uranyl acetate. Grids were imaged using a Zeiss Libra 200FE transmission electron microscope, operated at 200 kV.

### Agarose Gel Electrophoresis

To confirm DOX attachment, PVX−DOX filaments were run in a 0.8% (w/v) agarose gel (in TBE). PVX−DOX and corresponding amounts of free DOX or PVX alone were loaded with 6× agarose loading dye. Samples were run at 100 V for 30 min in TBE. Gels were visualized under UV light and after staining with 0.25% (w/v) Coomassie blue.

### Cell Culture and Cell Viability Assay

B16F10 cells (ATCC) were cultured in Dulbecco's modified Eagle's media (DMEM, Life Technologies), supplemented with 10% (v/v) fetal bovine serum (FBS, Atlanta Biologicals) and 1% (v/v) penicillin-streptomycin (penstrep, Life Technologies). Cells were maintained at 37° C., 5% CO₂. Confluent cells were removed with 0.05% (w/v) trypsin-EDTA (Life Technologies), seeded at 2×10³ cells/100 μL/well in 96-well plates, and grown overnight at 37° C., 5% CO₂. The next day, cells were washed 2 times with PBS and incubated with free DOX or PVX−DOX corresponding to 0, 0.01, 0.05, 0.1, 0.5, 1, 5, or 10 μM DOX for 24 h, in triplicate. A PVX only control corresponded to the amount of PVX in the highest PVX−DOX sample. Following incubation, cells were washed 2 times to remove unbound DOX or particles. Fresh medium (100 μL) was added and cells were returned to the 21 incubator for 48 h. Cell viability was assessed using an MTT proliferation assay (ATCC); the procedure was as the manufacturer suggested.

### Animal Studies

All Experiments Were Conducted in Accordance with Case Western Reserve University's Institutional Animal Care and Use Committee. C57BL/6J male mice (Jackson) were used. B16F10 tumors were induced intradermally into the right flank of C57BL/6J mice (1.25×10⁵ cells/50 μL media). Animals were monitored and tumor volume was calculated as V =0.5×a×b2, where a=width of the tumor and b=length of the tumor. Animals were sacrificed when tumor volume reached >1000 mm3. Treatment schedule: Eight days post-tumor induction (day 0), mice were randomly assigned to the following groups (n=3): PBS, PVX, or CPMV. Mice were treated intratumorally (20 μL), every 7 days, with 5 mg kg−1 PVX or CPMV. Mice were sacrificed when tumors reached a volume >1000 mm³. For chemo-immunotherapy combination therapy, mice were randomly assigned to the following groups (n=6): PBS, PVX, DOX, conjugated PVX−DOX, or PVX+DOX mixtures. PVX+DOX samples were prepared less than 30 min before injections and are considered not bound to each other. Mice were treated intratumorally (20 μL), every other day, with 5 mg kg⁻¹ PVX or PVX−DOX or the corresponding dose of DOX (˜0.065 mg kg−1). Mice were sacrificed when tumors reached a volume>1000 mm³.

### Immunostaining

When tumor reached volumes <100 mm³, mice were randomly assigned to the following groups (n=3): PBS or PVX−DOX. Mice were treated intratumorally (20 μL), every 7 days, with 5 mg kg−1 PVX−DOX. Mice were sacrificed when tumors reached a volume>1000 mm3 tumors were collected for analysis. Tumors were frozen in optimal cutting temperature compound (Fisher). Frozen tumors were cut into 12 μm sections. Sections were fixed in 95% (v/v) ethanol for 20 minutes on ice. Following fixation, tumor sections were permeabilized with 22 0.2% (v/v) Triton X-100 in PBS for 2 min at room temperature for visualization of intracellular markers. Then, tumor sections were blocked in 10% (v/v) GS/PBS for 60 min at room temperature. PVX and F4/80 were stained using rabbit anti-PVX antibody (1:250 in 1% (v/v) GS/PBS) and rat anti-mouse F4/80 (1:250 in 1% (v/v) GS/PBS) for 1-2 h at room temperature. Primary antibodies were detected using secondary antibody staining: AlexaFluor488-labeled goat-anti-rabbit antibody (1:500 in 1% (v/v) GS/PBS) and AlexaFluor555-labeled goat-anti-rat antibody (1:500 in 1% (v/v) GS/PBS) for 60 min at room temperature. Tumor sections were washed 3 times with PBS in between each step. Following the final wash, coverslips were mounted using Fluoroshield with DAPI. Slides were imaged on a Zeiss Axio Observer Z1 motorized FL inverted microscope. Fluorescence intensity was analyzed using ImageJ 1.47d.

### Luminex Assay

Intradermal melanomas were induced in C57BL/6J male mice (Jackson) as described above. Eight days post-tumor induction (day 0), mice were randomly assigned to the following groups (n=4): PBS, PVX, PVX−DOX, or PVX+DOX. Mice were treated intratumorally (20 μL) once and tumors were harvested at 24 h.p.i. Tumors were weighed and homogenized in T-PER™Buffer (ThermoFisher) at 1 mL of buffer/100 mg of tissue. TPER ™Buffer was supplemented with complete™ Protease Inhibitor Cocktail tablets (Roche) at one tablet per 8 mL of Buffer. After homogenization, homogenizer was rinsed with 0.5 mL HBSS (ThermoFisher) and added to the homogenate. Homogenate was centrifuged at 9,000×g for 10 minutes at 2-8° C. Supernatants were frozen and kept at −80° C. until analyses. Millipore Milliplex MAP mouse 32-plex was run at the CRWU Bioanalyte Core.

### Results

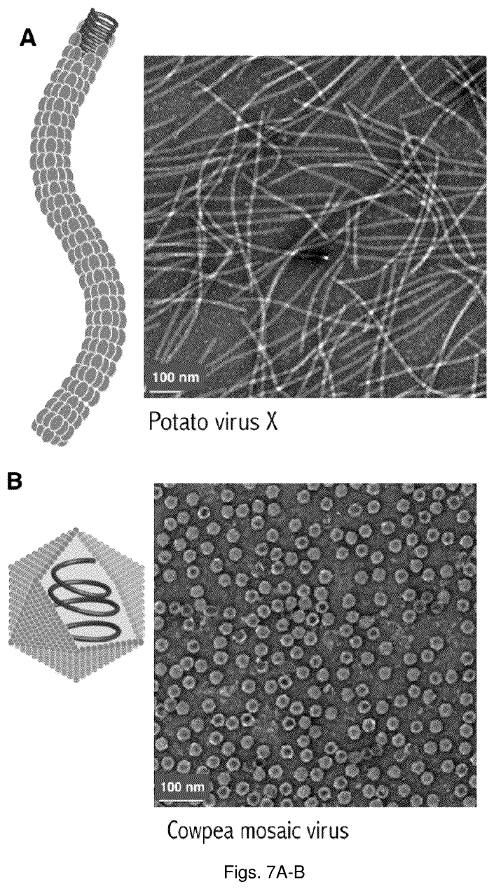

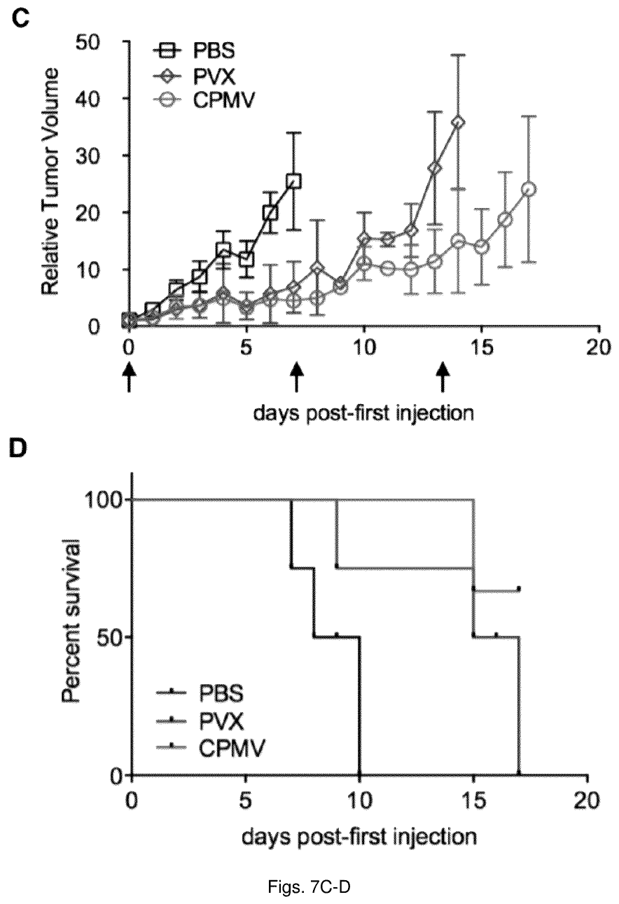

PVX is a filamentous plant virus, measuring 515×13 nm, and is comprised of 1270 identical coat proteins. While different in its physical nature compared to the 30 nm-sized icosahedrons formed by CPMV, PVX and PapMV share similar organization of the nucleoproteins arranged as flexuous soft matter filaments. To test whether PVX would stimulate an anti-tumor response when used as an in situ vaccine, we used the B16F10 melanoma model. B16F10 is a highly aggressive and poorly immunogenic tumor model used extensively for immunotherapy studies; it also has served as a model for the evaluation of the immunotherapeutic potential of virus-based therapies. Its low immunogenicity makes it an attractive platform to investigate new immunostimulatory therapies. B16F10 isografts were induced intradermally on the right flank of C57BL/6J mice. Eight days post-induction (tumor starting volume <100 mm³), mice were randomized (n=3) and treated weekly intratumorally with PBS or 100 μg of PVX or CPMV. Tumor volumes were measured daily and mice were sacrificed when tumors reached >1000 mm³. Treatment with CPMV or PVX alone significantly slowed tumor growth rate and extended survival time compared to PBS (FIG. 7), but there was no significant difference between CPMV and PVX treatment. These data indicate that PVX, like CPMV, can stimulate an anti-tumor response when used as an in situ vaccine.

Having established that PVX in situ vaccination slows tumor growth, we went ahead with a chemo-immuno combination therapy approach. We hypothesized that the combination of chemotherapy delivery, either co-administered (as physical mixture, PVX+DOX) or co-delivered (as complexed version, PVX−DOX), would enhance the anti-tumor effect. The underlying idea was that the chemotherapy would debulk the tumor to provide a burst of tumor antigens in the context of immunogenic cell death. This fosters specific immune recognition and response to those antigens; in turn, VLP-mediated immune-stimulation would further augment anti-tumor immunity and induce memory to protect from outgrowth of metastases and recurrence of the disease.

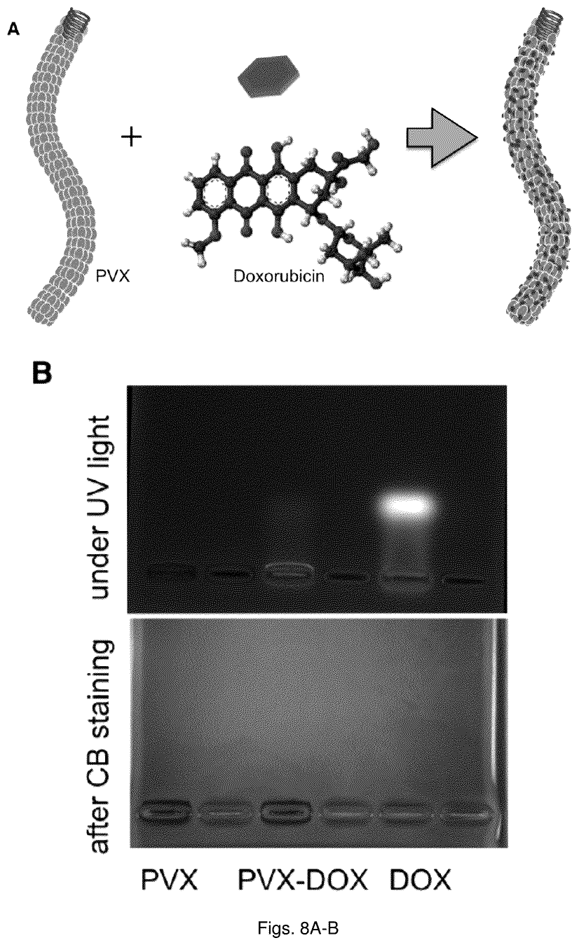

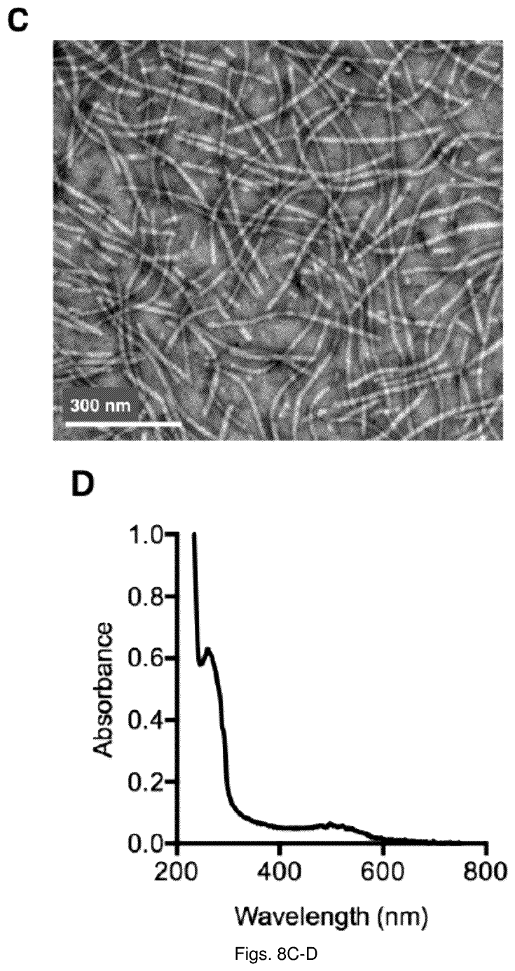

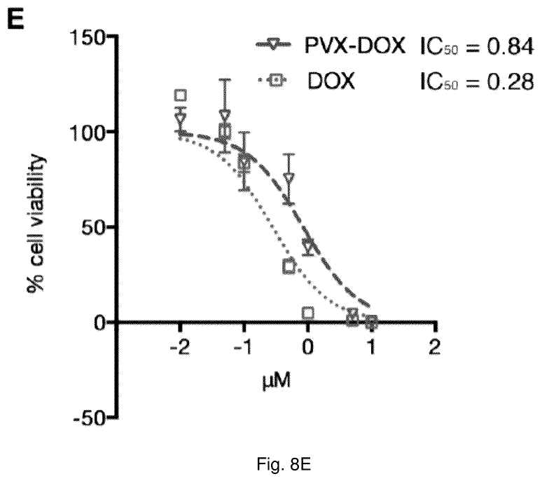

To obtain the PVX−DOX complex (FIG. 8A), purified PVX was loaded with DOX by incubating a 5,000 molar excess of DOX with PVX for 5 days; excess DOX was removed by ultracentrifugation. Incubation criteria were optimized: increasing molar excess of DOX resulted in extensive aggregation and further increasing incubation time did not increase loading capacity (data not shown). The PVX−DOX complex was characterized by agarose gel electrophoresis, UV/visible spectroscopy, and transmission electron microscopy (TEM) (FIGS. 8B-D). TEM imaging confirmed particle integrity following DOX loading. The PVX−DOX formulation stability was confirmed after 1 month of storage at 4° C.; DOX release was not apparent and the particles remained intact (data not shown). UV/visible spectroscopy was used to determine the number of DOX attached per PVX. The Beer-Lambert law, in conjunction with PVX- and DOXspecific extinction coefficients, was used to determine the concentrations of both PVX and DOX in solution. The ratio of DOX to PVX concentration was then used to determine DOX loading. Each PVX was loaded with ˜850 DOX per PVX. Agarose gel electrophoresis analysis indicated that DOX was indeed associated with PVX and not free in solution, as free DOX was not detectable in the PVX−DOX sample. The association of DOX with PVX may be explained based on hydrophobic interactions and π-π stacking of the planar drug molecules and polar amino acids.

Efficacy of the PVX−DOX complex was confirmed using B16F10 melanoma cells (FIG. 8E). DOX conjugated to PVX maintained cell killing ability, although with decreased efficacy resulting in an IC₅₀ value of 0.84 μM versus 0.28 μM for free DOX. Similar trends have been reported with synthetic and virus-based nanoparticles for DOX delivery. The reduced efficacy may be explained by reduced cell uptake and required endolysosomal processing when DOX is delivered by nanoparticles.

To test the hypothesis that a combination chemo-immunotherapy would potentiate the efficacy of PVX alone, DOX-loaded PVX (PVX−DOX) and PVX+DOX combinations were tested in the B16F10 murine melanoma model. The combination of PVX+DOX served to test whether merely the combination of the therapies or the co-delivery (PVX−DOX) would enhance the overall efficacy. PVX+DOX was combined less than 30 min before injection to ensure that the two therapies did not have time to interact. PBS, PVX alone, and free DOX were used as controls. When tumors were <100 mm3, mice were treated every other day intratumorally with PVX−DOX, PVX+DOX, or corresponding controls (n=6). Tumor volumes were measured daily and mice were sacrificed when tumors reached >1000 mm³. PVX was administered at 5 mg kg⁻¹ (corresponding to a dose of 0.065 mg kg⁻¹ DOX). Clinically, doxorubicin is administered at doses of 1-10 mg kg⁻¹, intravenously. If 1-10% of the injected dose reaches the tumor site, the resulting intratumoral dose would equate to 0.01-1 mg kg⁻¹; thus our intratumoral dose is within a clinically relevant range of DOX.

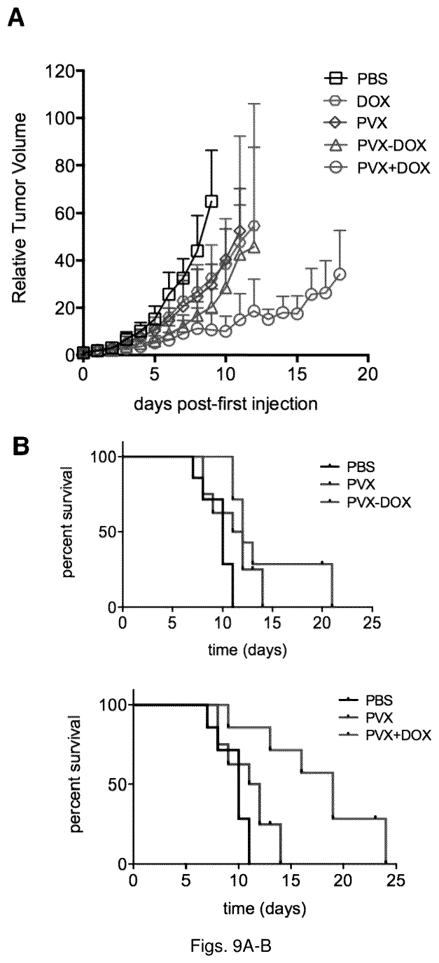

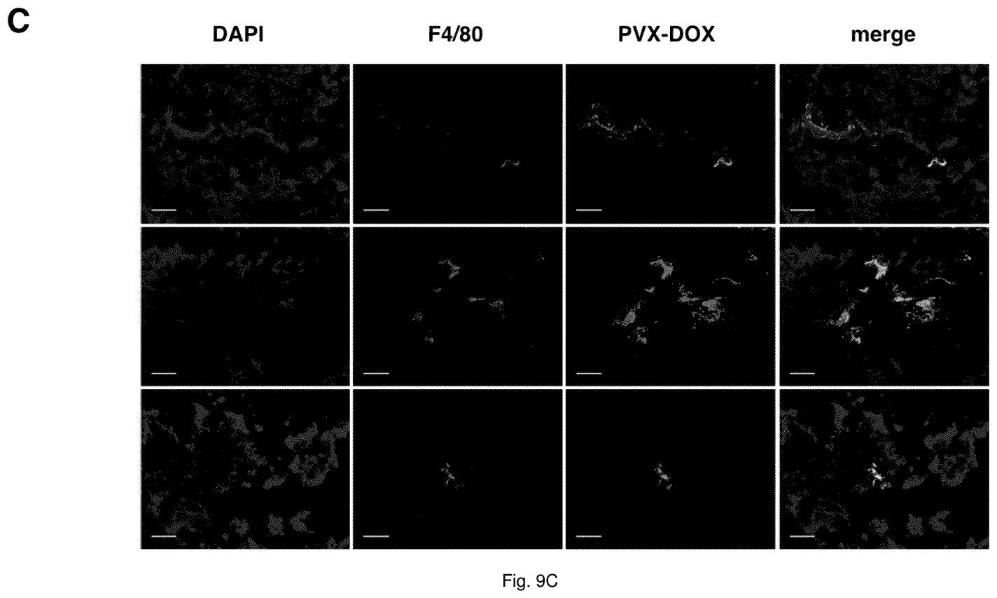

While there was no statistical difference in tumor growth rate or survival time between PVX−DOX complex versus PVX or DOX alone, PVX+DOX did significantly slow tumor growth rate versus PVX and DOX alone (FIGS. 9A+B). Thus, the data indicate that the combination of DOX chemotherapy and PVX immunotherapy indeed potentiates efficacy, however the formulation as a combined nanoparticle, PVX−DOX, did not improve the treatment. The lack of statistically significant enhancement of efficacy of the PVX−DOX complex versus immuno-or chemo-monotherapy may be explained by the fact that the therapies synergize best when they act on their own. DOX targets replicating cancer cells to induce cell death, and PVX likely associated with immune cells to stimulate an anti-tumor effect-most likely through activation of signaling cascades through pathogen-associated molecular pattern (PAMP) receptors and other danger signals. Indeed, we found that PVX was co-localized with F4/80+ macrophages within the tumor tissue (FIG. 9C), which may cause killing of immune cells rather than cancer cells; even if the nanoparticles do not exhibit cytotoxic effect on the immune cell population, the sequestration of PVX−DOX in the immune cells would lower the anti-tumor efficacy of the complex.

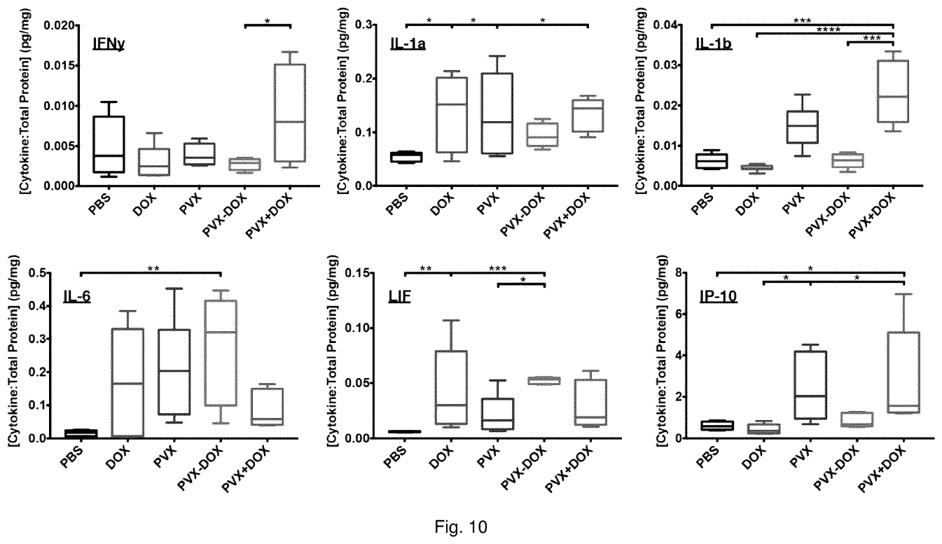

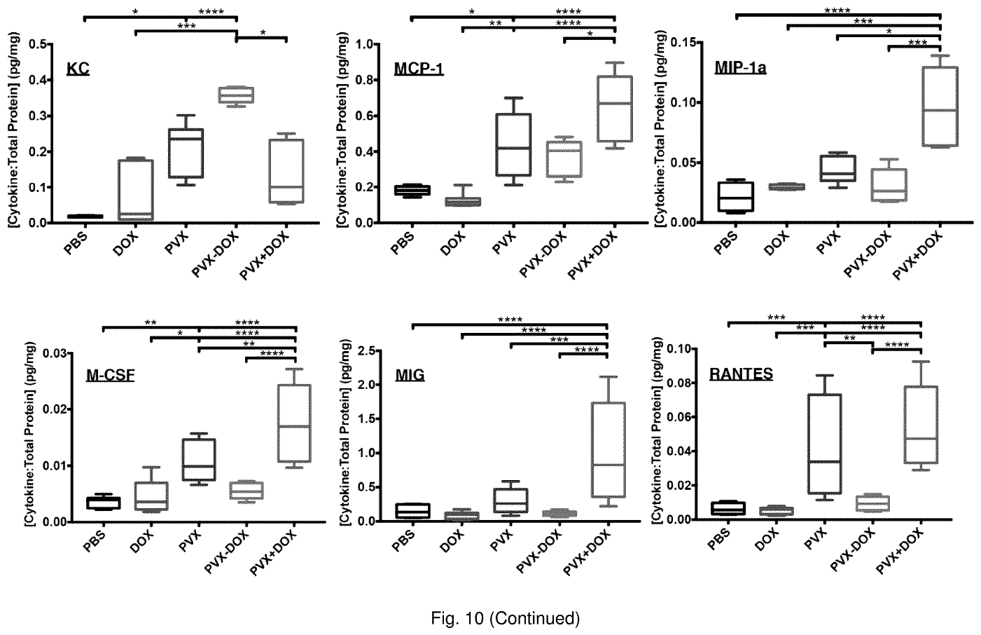

To gain insight into the underlying immunology we performed cytokine/chemokine profiling using a 32-plex MILLIPLEX® Luminex® assay. Tumors were treated with PBS, PVX, DOX, PVX−DOX, or PVX+DOX and harvested 24 hours after the first injection. Profiles were obtained using tumor homogenates and normalized to total protein levels by the bicinchoninic acid (BCA) assay. The PVX+DOX group repeatedly showed significantly higher particular cytokine and chemokine levels compared to any other group (FIG. 10). Specifically, interferon gamma (IFNγ) and IFNγ-stimulated or synergistic cytokines were elevated. These included, but may not be limited to: Regulated on Activation, Normal T Cell Expressed and Secreted (RANTES/CCL5), Macrophage Inflammatory Protein 1a (MIP-1a/CCL3), Monocyte Chemoattractant Protein (MCP-1/CCL2), Monokine Induced by Gamma interferon (MIG/CXCL9), and IFNγ-induced protein 10 (IP-10). IFNγ is a multifunctional type II interferon critical for inducing a pro-inflammatory environment and antiviral responses and is often associated with effective tumor immunotherapy responses. Under the influence of IFNγ, these chemokines mediate the influx of monocytes, macrophages, and other immune cells. Interestingly, the induction of IFNγ was not associated with the increased expression of its master positive regulator, IL-12, thus in this context, the increased expression of IFNγ is IL-12-independent (data not shown). In the tumor microenvironment, activation of the IFNγ pathway is in accordance with other work, where viruses were applied as an in situ vaccine. Stimulation of the IFNγ pathway alleviates the immuno-suppressive tumor microenvironment promoting an anti-tumor immune response. The molecular receptors and signaling cascades are yet to be elucidated, but the body of data indicates IFNγ to be a key player for viral-based in situ vaccination approaches.

Other noteworthy cytokines/chemokines that were up-regulated include interleukin-1β (IL-1β) and Macrophage Colony-Stimulating Factor (M-CSF). IL-1β is known to be an early pro-inflammatory cytokine activated by many PAMPs and Danger Associated Molecular Patterns (DAMPs). IL-1β signaling was also observed in our earlier work with CPMV, and data suggest that initial recognition of the viral in situ vaccine by innate surveillance cells is promoting immune activation. IL-1β and M-CSF are both major recruiters and activators of monocytes and macrophages to the site of challenge. M-CSF, in particular, enhances monocyte functions including phagocytic activity and cytotoxicity for tumor cells, while inducing synthesis of inflammatory cytokines such as IL-1, TNFα, and IFNγ in monocytes. PVX monotherapy appears to follow a similar trend of increased expression of cytokines/chemokines, with further enhanced response through combination with DOX when coadministered (PVX+DOX), but reduced response when directly coupled together as PVX−DOX.

In this example, we demonstrated that PVX stimulates an anti-tumor immune response when used as an in situ vaccine. Data indicate that the plant virus-based nanoparticles activate the innate immune system locally—this innate immune activation is thought to overcome the immune suppressive tumor microenvironment re-starting the cancer immunity cycle leading to systemic elimination of cancer cells through the adaptive immune system. It is likely that innate receptors such as pattern recognition receptors (PRR) play a key role recognizing the multivalent nature of the plant virus nanoparticles; the repetitive, multidentate coat protein assemblies are products known as pathogen-associated molecular patterns (PAMPs).

The combination of the DOX chemotherapeutic with PVX was more efficacious than the monotherapies when co-administered as the PVX+DOX formulation, but not when physically linked in the PVX−DOX formulation. Data show that PVX+DOX induced a higher immune mediator profile within the tumor microenvironment, in turn resulting in increased efficacy against B16F10 melanoma. A key conclusion to draw from these studies is that the combination of chemo- and immunotherapy indeed is a powerful tool—yet the formulation of the two regimes into a single, multi-functional nanoparticle may not always be the optimal approach.

It has long been recognized that nanoparticles are preferentially ingested by phagocytic cells. FIG. 9C confirms this and shows that PVX−DOX is concentrated in F4/80+ macrophages within the tumor. The basis for reduced efficacy of PVX−DOX as compared to PVX+DOX could simply be because when phagocytes ingest PVX−DOX, it reduces the concentration of DOX available to react with tumor cell DNA. If as seems likely, the cells that respond to PVX at least initially are phagocytes, then a nonexclusive alternative could be that ingestion of PVX−DOX by phagocytes leads to a different response than ingest of PVX by itself by those cells, thus blunting the immune response stimulated by PVX.

### Example 5

This example describes the use of PVX for delivery of doxorubicin (DOX), an FDA-approved anti-tumor drug used in treatments of various malignancies including breast cancer, ovarian cancer, acute lymphoblastic leukemia, Hodgkin's lymphoma, and small cell lung cancer. DOX is an anthracycline that kills cancer cells via three mechanisms of action: (1) intercalating with DNA, (2) inhibiting topoisomerase II, and (3) producing reactive oxygen species. However, like all chemotherapies, when administered systemically, it is associated with off-target effects due to non-specific interaction with healthy cells, ranging in severity from hair loss to heart failure. Herein, we loaded DOX onto the surface of PVX by hydrophobic interactions and characterized the therapeutic nanoparticles (DOX-loaded PVX) using transmission electron microscopy, gel electrophoresis, and UV/visible spectroscopy. The efficacy of the approach was then examined in a panel of cancer cells in vitro. Lastly, we assessed efficacy using a mouse model of triple negative breast cancer using MDA-MB-231 breast cancer xenografts in athymic mice.

### Materials and Methods

### Preparation of PVX

PVX was propagated in Nicotiana benthamiana plants and purified as previously reported.

### Preparation and Characterization of DOX-Loaded PVX

Doxorubicin HCl (denoted as DOX) was purchased from INDOFINE Chemical Company. To prepare PVX−DOX, PVX (at 2 mg mL⁻¹ in 0.1 M potassium phosphate buffer (KP), pH 7.0) was incubated with a 5000 molar excess of DOX in a 10% (v/v) final concentration of DMSO for 5 days at room temperature, with agitation. PVX−DOX was purified twice over a 30% (w/v) sucrose cushion using ultracentrifugation (212,000×g for 3 h at 4° C.) and resuspended overnight in 0.1 M KP, pH 7.0. The particles were stored in 0.1 M KP, pH 7.0 at 4° C. until used.

To prepare therapeutic particles for the in vivo study, we also conjugated polyethylene glycol (PEG) using PEG-succinimidyl ester with a molecular weight of 5000 Da (PEG-NHS, purchased from Nanocs) to formulate PEG-PVX−DOX. PEG-NHS was added to the mixture of PVX and DOX on day 4 at 2000 molar excess of PVX and incubated overnight at room temperature. Following purification as described above, PVX, PVX−DOX, and PEG-PVX−DOX were characterized by transmission electron microscopy (TEM), agarose gel electrophoresis, denaturing gel electrophoresis (SDS-PAGE), and UV/visible spectroscopy.

Transmission electron microscopy (TEM) was performed after DOX loading to confirm integrity of PVX−DOX filaments. PVX−DOX samples (0.1 mg mL⁻¹, in dH₂O) were placed on carbon-coated copper grids and negatively stained with 0.2% (w/v) uranyl acetate. Grids were imaged using a Zeiss Libra 200FE transmission electron microscope, operated at 200 kV.

To confirm PVX−DOX association, PVX−DOX filaments were separated using a 0.8% (w/v) agarose gel (in 1× TBE buffer). PVX−DOX or PEG-PVX−DOX and corresponding amounts of free DOX or PVX alone were loaded with 6× agarose loading dye (Fisher Scientific).

Samples were run at 100 V for 30 min in TBE. Gels were visualized under UV light and white light before and after staining with 0.25% (w/v) Coomassie Blue (CB) staining, respectively.

SDS-PAGE was carried out to determine the number of PEG chains per PVX filament. 10 μg of denatured protein samples were analyzed on 4-12% NuPage gels (Life Technologies) in 1×MOPS SDS running buffer (Life Technologies). Protein bands were visualized under UV light and white light before and after staining with CB. Band density analysis was conducted using ImageJ 1.47d.

The number of DOX per PVX filament was determined by UV/visible spectroscopy using a NanoDrop 2000 spectrophotometer. PVX concentration and DOX loading were determined using the Beer-Lambert law and the DOX-(11,500 M⁻¹ cm⁻¹ at 495 nm) and PVX-specific (2.97 mL mg ⁻¹ cm⁻¹ at 260 nm) extinction coefficients.

### Cell Culture

A2780, a gift from Dr. Analisa DiFeo (Case Western Reserve University) were cultured in Dulbecco's modified Eagle's media (DMEM, Life Technologies), supplemented with 10% (v/v) fetal bovine serum (FBS, Atlanta Biologicals) and 1% (v/v) penicillin-streptomycin (penstrep, Life Technologies). HeLa (ATCC) were cultured in minimum essential media (MEM, Life Technologies), supplemented with 10% (v/v) FBS, 1% (v/v) penstrep, and 1% (v/v) L-glutamine (Life Technologies). MDA-MB-231, a gift from Dr. Ruth Keri (Case Western Reserve University), were cultured in Roswell Park Memorial Institute (RPMI, Life Technologies) medium supplemented with 10% (v/v) FBS, 1% (v/v) penstrep, and 1% (v/v) L-glutamine. All cells were maintained at 37° C. and 5% CO₂.

### Cytotoxicity Evaluation

Confluent cells were removed with 0.05% (w/v) trypsin-EDTA (Life Technologies), seeded at either 2×10³ cells/100 μL/well (MDA-MB-231 and HeLa) or 5×10³ cells/100 μL/well (A2780) in 96-well plates and grown overnight at 37° C., 5% CO₂. The next day, cells were washed 2 times with PBS and incubated with free DOX, PVX−DOX, or PEG-PVX−DOX corresponding to 0, 0.01, 0.05, 0.1, 0.5, 1, 5, or 10 μM (A2780, MDA-MB-231) or 0, 0.1, 0.5, 1, 5, 10, 25, or 50 μM (HeLa) DOX for 24 h, in triplicate. A PVX only control corresponded to the amount of PVX in the highest PVX−DOX sample. Following incubation, cells were washed 2 times to remove unbound DOX or particles. Fresh medium (100 μL) was added and cells were returned to the incubator for 48 h. Cell viability was assessed using an MTT proliferation assay (ATCC); the procedure was as the manufacturer suggested.

### Tracking of Free DOX and PVX−DOX in Cells

A2780 cells were grown to confluency and removed with 0.05% (w/v) trypsin-EDTA. Cells were seeded at 1×10⁴ cells/500 μL/well on coverslips in untreated 24-well plates and grown overnight at 37° C., 5% CO₂. The next day, cells were washed 3 times and incubated with free DOX or PVX−DOX (1.5 μM DOX) for 2, 4, 12, 24, or 48 h. Following incubation, cells were washed 3 times and fixed in DPBS (Corning) containing 5% (v/v) paraformaldehyde (Electron Microscopy Sciences) and 0.3% (v/v) glutaraldehyde (Polysciences) for 10 min at room temperature. Coverslips were mounted using Fluoroshield (Sigma). Slides were imaged on a Zeiss Axio Observer Z1 motorized FL inverted microscope. Fluorescence intensity was analysed as follows using ImageJ 1.47d. All images were identically processed prior to fluorescence intensity measurement. Nuclei were selected and analyzed for fluorescence intensity using the measure feature of ImageJ. For each sample, a minimum of 3 images was analyzed and at least 4 cells were analyzed per image.

### In Vivo Efficacy of PEG-PVX−DOX

All experiments were conducted in accordance with Case Western Reserve University's Institutional Animal Care and Use Committee. Female NCR nu/nu mice (6 weeks old) were injected subcutaneously into the right flank with 2×10⁶ MDA-MB-231 cells suspended in 100 μL of media and Matrigel (Corning) at a 1:1 ratio. When tumors reached 100-200 mm³, a dosage of 1.0 mg/kg body weight DOX was injected intravenously (this dosage corresponds to 1.0-2.0 mg PVX depending on batches). Groups of 4 animals each were treated with PEG-PVX−DOX, free DOX, or PBS. The dosage was normalized to the DOX content. Treatments were performed every 5 days, and a total of 5 treatments was given.

Tumors were measured daily and total volume was calculated using the formula: v=(1×w²)/2. Mice were euthanized following 25 days of treatment.

### Results

### Synthesis and Characterization of DOX-Loaded PVX

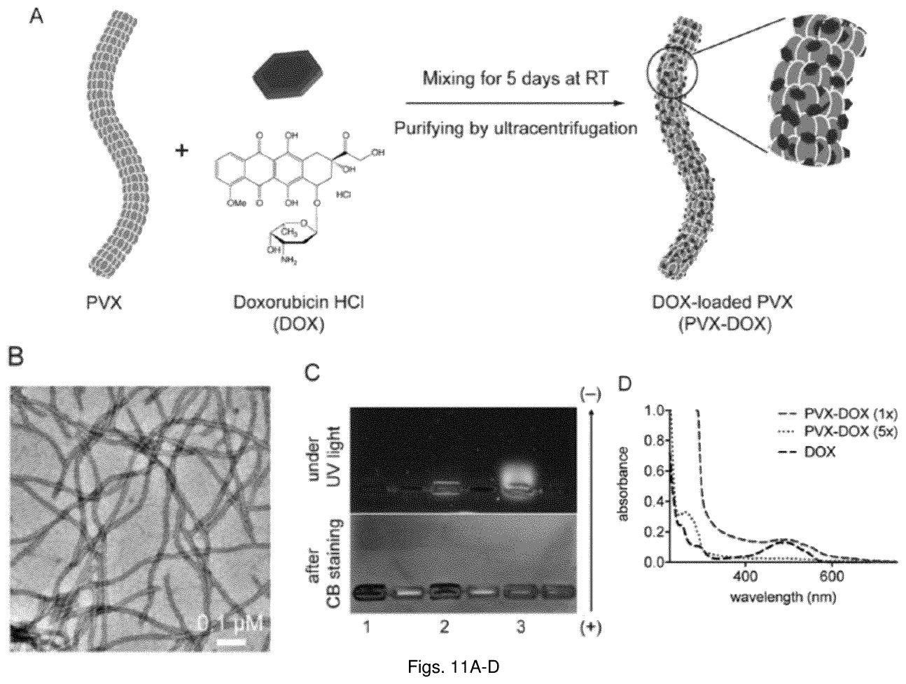

PVX was produced through farming in N. benthamiana plants and purified using established protocols. From 100 g of infected leaf material, about 20 mg of pure PVX was extracted. DOX was loaded onto PVX by incubating excess DOX with the filaments in 0.1 M KP buffer (pH 7.0, final 10% (v/v) DMSO) for 5 days at room temperature (FIG. 11A). We used a 5000 molar excess of DOX per PVX; this was determined to be the optimized ratio that allowed homogenous mixing without aggregation. Further prolonged incubation time did not increase loading capacity (data not shown). The reaction was purified twice via ultracentrifugation. Purified DOX-loaded PVX (denoted as PVX−DOX) was characterized by TEM, agarose gel electrophoresis, and UV/visible spectroscopy (FIG. 11B-D).

First, TEM was used to confirm the particle integrity after loading DOX. The filamentous structure remained unchanged as seen from the TEM image (FIG. 11B). Agarose gel electrophoresis was used to confirm that DOX was associated to PVX and not free in solution. DOX is fluorescent when visualized under UV light and PVX was visualized under white light after Coomassie Blue (CB) staining. PVX is too large to migrate through the gel in 30 min, and remains confined to the well. Under UV light, no signal was seen in the PVX only lane, while PVX−DOX signal was seen directly within the well (FIG. 11C). The free DOX control, due to its positive charge, migrated towards the cathode. Imaging of the gels after staining with CB confirmed the presence of PVX and PVX−DOX in their respective wells; gel electrophoresis also indicates that DOX is stably associated with PVX. Finally, UV/visible spectroscopy was used to determine the number of DOX associated per PVX. The Beer-Lambert law, in conjunction with the PVX-and DOX-specific extinction coefficients, was used to determine the concentrations of both PVX and associated DOX in solution. The ratio of DOX to PVX concentration was then used to determine DOX loading. It was determined that PVX was loaded with 850-1000 DOX (FIG. 11D); the range indicates batch-to-batch variability. Noteworthy, the peak derived from DOX in PVX−DOX was red-shifted, indicating the association of DOX to PVX (FIG. 11D). PVX−DOX stability was confirmed after 1 month of storage at 4° C.; DOX release was not apparent (data not shown).

### PVX−DOX Maintains Cell Killing Ability In Vitro

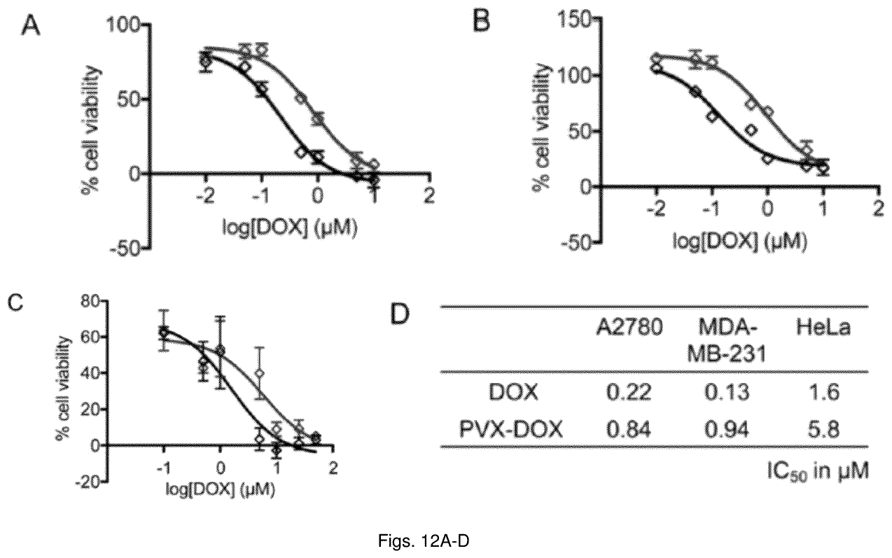

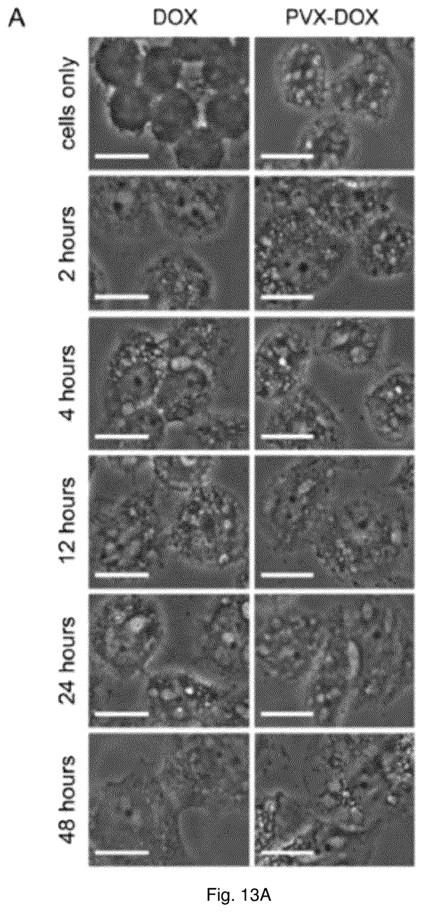

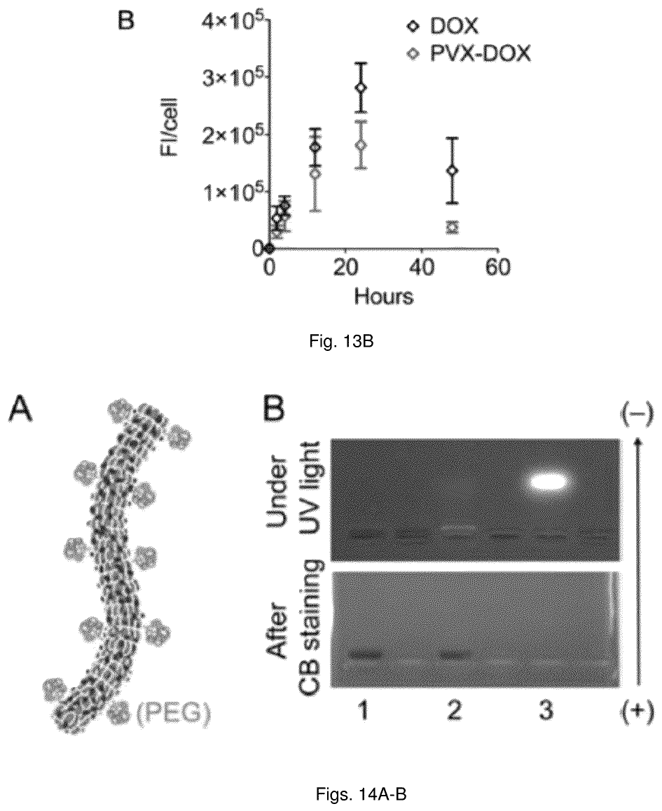

We next determined whether doxorubicin maintained its cell killing ability after being loaded onto PVX. Increasing amounts of PVX−DOX and corresponding free DOX were evaluated in a panel of cell lines: A2780 (ovarian cancer), MDA-MB-231 (breast cancer), and HeLa (cervical cancer) (FIG. 12A-D). The calculated IC₅₀ values of PVX−DOX were 0.84, 0.94, and 5.8 μM for A2780, MDA-MB-231, and HeLa, respectively (FIG. 12D).

Meanwhile, free DOX showed slightly lower values (indicating higher efficacy) at each corresponding cell line: 0.22, 0.13, and 1.6 μM (FIG. 12D). The calculated IC⁵⁰ values for free DOX are similar to previously reported values. PVX itself at the highest corresponding concentration exhibited no toxicity, thus attributing the cell killing function to the doxorubicin activity. A similar trend was observed in all tested cell lines: PVX−DOX was therapeutically active, but with decreased efficacy compared to free DOX (p<0.05).

### Tracking of DOX and PVX−DOX in A2780 Cell Line

The decreased efficacy of PVX−DOX vs. free DOX could be due to decreased uptake or inefficient release and trafficking of the PVX−DOX complex. To address these questions, we performed fluorescence microscopy studies to monitor DOX accumulation in the nucleus of treated cells. A2780 cells were incubated with 1.5 M of DOX (either as PVX−DOX or free DOX) for 2, 4, 12, 24, or 48 h and imaged using a fluorescence microscope. DOX fluorescence per cell was analysed using ImageJ software (FIG. 13). As reported in previous studies, DOX accumulated within the nucleus, where it was visible via fluorescence. DOX within the cytoplasm is not fluorescent due to scattering and absorption by cellular components. Additionally, DOX is more concentrated within the nucleus than when it is dispersed throughout the cytoplasm; increased concentration of DOX is correlated to increases in fluorescence intensity. Up until 24 h, the accumulation of DOX in the nucleus increased over time, as shown by increased fluorescence observed in cells treated with either PVX−DOX or free DOX (FIG. 13A). At 48 h, treated cells were undergoing apoptosis leading to reduced DOX fluorescence-drug-induced apoptosis was apparent for cells treated with either PVX−DOX or free DOX attesting to the efficacy of the PVX-based delivery system.

Quantitative analysis of the images indicated that PVX−DOX exhibited lower nuclear accumulation compared to free DOX at all time points tested (FIG. 13B). However, the decrease was only statistically significant (p<0.05) at 24 and 48 h.

### Preparation of PEGylated PEG-PVX−DOX Nanoparticles

To assess the efficacy of PVX−DOX in vivo, we prepared a PEGylated formulation. Like other nanoparticles, PVX is cleared by the mononuclear phagocyte system (MPS) when administered systemically. Conjugation of stealth polymers such as PEG, however, decreases serum protein adsorption, resulting in the “stealth properties” that enhance circulation time and decrease accumulation in liver and spleen. We previously reported that PEGylated PVX coated with linear PEG chains with a molecular weight of 5000 Da demonstrates enhanced in vivo fates and favorable tumor accumulation.

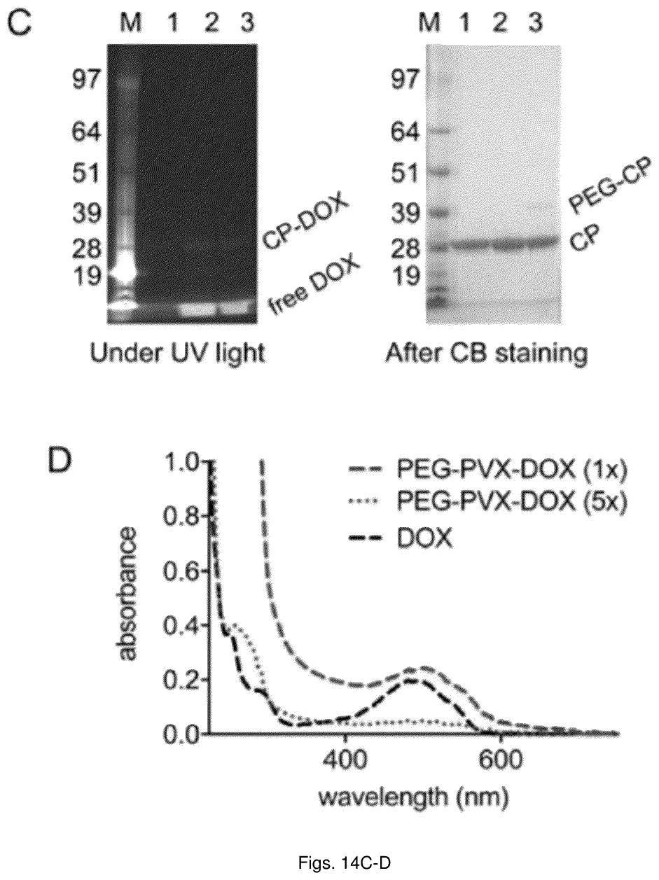

To prepare PEG-PVX−DOX, PEG-NHS (using a 2000 molar excess of PEG to PVX) was added into the mixture of PVX and DOX on day 4 (FIG. 14A). Excess DOX and PEG were removed by two rounds of ultracentrifugation. We used agarose gel and denaturing gel (SDS-PAGE) electrophoresis to confirm the loading of DOX and attachment of PEG. Agarose gel electrophoresis indicated that free DOX was completely removed from the solution and the fluorescence was only derived from DOX associated with PVX (FIG. 14B). Additionally, SDS-PAGE (FIG. 14C) confirmed successful conjugation of PEG to the PVX coat protein. Under UV light, DOX, released from its PVX carrier, is detectable at the bottom of gel. Nevertheless, dim fluorescence was observed from the PVX coat proteins (˜25 kDa) indicating that some amounts of DOX remained associated with the protein even after exposure to denaturing conditions and electrophoretic separation. Analysis by ImageJ indicates that 95-97% of DOX was released from the denatured PVX. After staining by CB, the PVX coat protein and an additional band at a higher molecular weight corresponding to the PEGylated coat protein was observed in PEG-PVX−DOX but not PVX−DOX. To estimate the degree of PEGylation (number of PEG chains per PVX particle), band density analysis was performed using ImageJ. About 10% of PVX coat proteins were modified with PEG. Finally, the UV/visible spectrum indicated that about 1000-1500 DOX were loaded per particle (FIG. 14D); the range indicates the batch-to-batch variability. A red shift of the DOX-derived absorbance peak was also observed for PEG-PVX−DOX (as in PVX−DOX), indicating the association of DOX with the PEGylated PVX (FIG. 14D). Data show that higher loading of DOX to the PEGylated vs. native PVX is achieved. The increase in DOX loading might be because attached PEG chains further entrapped free DOX.

### In Bitro and In Vivo Efficacies of PEG-PVX−DOX

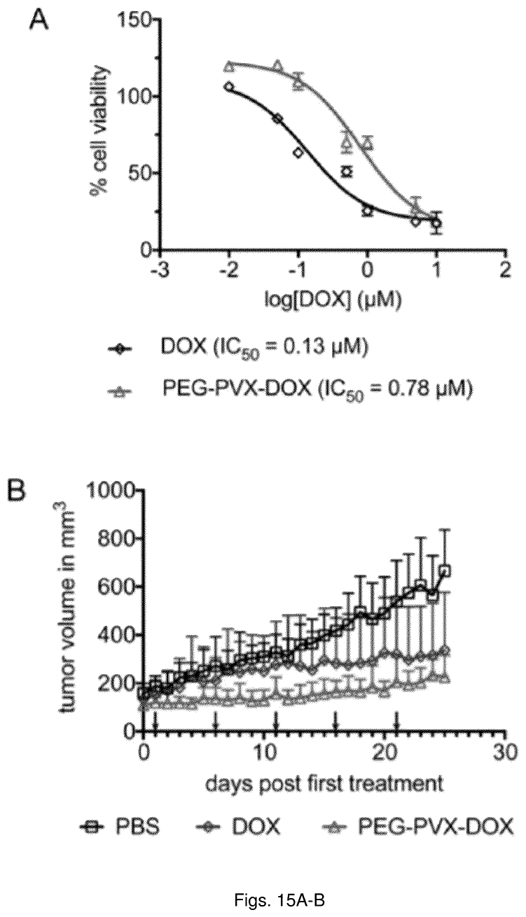

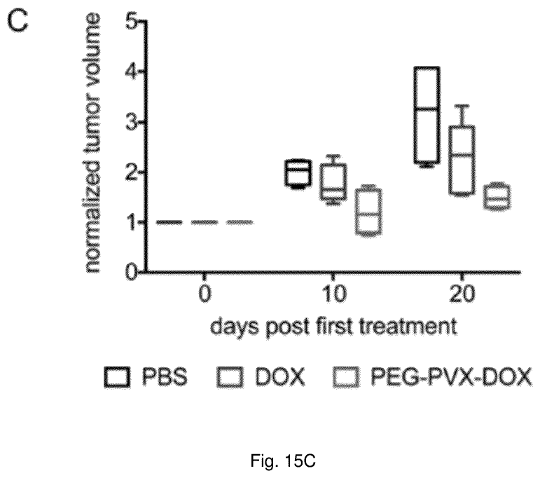

We chose a model of triple negative breast cancer to assess the efficacy of PEG-PVX−DOX. First, in vitro efficacy was tested using MDA-MB-231 cells to confirm cell killing of the PVX-based drug carrier after PEGylation. Increasing amounts of PEG-PVX−DOX and free DOX were added to the culture media for 24 h then cells were washed and efficacy determined using an MTT assay. Analogous to PVX−DOX, PEG-PVX−DOX had cell killing ability but decreased efficacy compared to free DOX (FIG. 15A). The efficacy of PEG-PVX−DOX was 0.78 μM, which is about 6-fold higher than that of free DOX (0.13 μM). There is no significant difference in efficacies with and without PEG (FIG. 12D and 15A).

Next, we investigated the capacity of PEG-PVX−DOX to inhibit tumor growth using MDA-MB-231 tumor-bearing athymic mouse model. Treatments were started when the tumor volume reached 100-200 mm³. The dosage of DOX on the carrier as well as free DOX was 1 mg/kg body weight. Intravenous injections into the tail vein were performed every 5 days for a total of 5 injections. Tumor volumes were measured daily. Statistically significant differences in mice treated with PBS or free DOX were only observed after day 22 post first treatment (FIG. 15B). In contrast, statistical significant differences between PBS vs PEG-PVX−DOX were apparent from day 7 post first treatment (p<0.05) (FIG. 15B). At the end of the treatment, tumor volumes in PEG-PVX−DOX treated group were 2.6 times smaller, while ones in DOX treated group were 2.1 times smaller than the PBS control. While data did not show statistical significance between DOX vs. PEG-PVX−DOX at the end of the treatment, a trend was apparent indicating that tumor volumes were smaller when mice were treated with PEG-PVX−DOX compared to the controls; this trend was observed throughout the study and indicates effective inhibition of tumor growth mediated by the plant virus-based drug delivery formulation (FIG. 15B,C).

We tested the efficacy of PVX−DOX in vitro in a panel of DOX-sensitive cell lines: A2780 (ovarian cancer), MDA-MB-231 (breast cancer), and HeLa (cervical cancer) using the MTT assay. There was a similar trend in these models: PVX−DOX exhibited cell killing, but with decreased efficacy compared to free DOX. However, these results are not unexpected; similar trends have been reported with synthetic and virus-based nanoparticles for DOX delivery. The reason for the lower efficacy may be explained by the lower uptake of DOX in the nucleus when delivered as PVX−DOX vs. free DOX (as was shown through longitudinal cell imaging using A2780 cells, see FIG. 13A). Differences in DOX uptake could be explained by uptake mechanisms of free DOX versus PVX-delivered DOX. Free DOX is taken up via diffusion across the cell membrane, while VNPs such as PVX are likely taken up by endocytosis or macropinocytosis. Thus, it is possible that PVX-delivered DOX is not as readily taken up as free DOX, resulting in decreased accumulation over time. Indeed, nuclear fluorescence intensity from PVX−DOX is approximately ⅔ of the nuclear fluorescence intensity measured for free DOX after 24 h of incubation. In comparison, DOX killed A2780 cells 4 times more effectively than PVX−DOX. The non-linear relationship between the imaging results and the MTT assay may be explained by the differences in experimental set up: nuclear fluorescence intensity was determined 24 h post-exposure to DOX or PVX−DOX; however, for cell viability, cells were incubated with DOX or PVX−DOX for 24 h followed by an additional 48 h incubation time. Thus, the differences between DOX and PVX−DOX may be more profound in the MTT assay vs. the imaging study.

Nevertheless, the in vitro assays do not reflect the complexity of the in vivo situation. In vivo additional biological barriers must be considered—and biodistribution and clearance for free and PVX-formulated DOX will not follow the same trend. The conclusion to be drawn from the in vitro studies are that even though the IC₅₀ indicates reduced efficacy of the nanoparticle formulation vs. free DOX, the PVX−DOX formulations maintains cell killing efficacy.

Finally, we prepared the stealth therapeutic nanoparticle PEG-PVX−DOX for evaluation of the in vivo efficacy in the MDA-MB-231-bearing athymic mouse model. PEGylation did not alter the cell killing ability (FIG. 15A). In a mouse model of triple negative breast cancer, we observed that PEG-PVX−DOX outperformed free DOX (FIG. 15B,C), highlighting the potential of PVX as a drug carrier.

### Example 6

Diffuse large B cell lymphoma (DLBCL) is the most common histological subtype of non-Hodgkin lymphomas (NHL) and affects both male and female regardless of age; current standard of care consists of chemoimmunotherapy R-CHOP (rituximab, cyclophosphamide, doxorubicin, vincristine, and prednisone), resulting in a good response and overall survival. However, depending on the stage, some patients (˜30-40%) suffer from resistance/relapse, likely due to the heterogeneous and aggressive nature of this malignancy and toxicities associated with the chemotherapy regimen. Alternatively, immunotherapy, particularly monoclonal antibodies (mAbs), have been used as an effective treatment option; albeit their intrinsic targeting properties, mAbs shown critical drawbacks associated with their extended half-life, leading to toxicity in non-targeted tissues and lack or poor response from treated patients. Hence, seek for new efficacious and safer therapeutic strategies for B cells neoplasia are demanding.

Plant viruses are naturally occurring biological nanomaterials that offer potential biomedical applications due to their intrinsic immunogenicity properties, acting as adjuvants in vaccines and immunotherapies. Particularly, cowpea mosaic virus (CPMV) has demonstrated remarkable antitumor efficacy as intratumoral immunotherapy in several mouse models and in companion dogs cancer patients. Unlike oncolytic virus (OVs), plant viruses are non-infectious and, consequently, non-replicating in mammalian cells; instead, plant viruses are immunogenic and agonize mammalian immune system through recognition by pattern recognition receptors (PRRs) in immune cells, particularly toll-like receptors (TLRs). Upon intratumoral administration, CPMV kickstart the cancer immunity cell cycle, acting as multivalent TLR agonist; the proteinaceous structure of CPMV capsid agonizes TLR2/4, while its ssRNA agonize TLR7 in the endosomes, enhancing the stimulation of antiviral inflammatory response, mainly lead by type I IFN (interferons), resulting in stimulation of immune cells activation and infiltration in the TME, repolarizing the tumor immune cells infiltrate (TILs) to anti-tumor phenotype. While interacting with innate immune cells, CPMV elicits systemic and long-lasting anti-tumor response by reestablishing the cancer immunity cell cycle, enhancing the tumor antigen presentation and eliciting CD8+ effector and memory T cells activation, leading to remission of injected and distant (non-injected) tumors.

PVX also act as an intratumoral immunotherapy (IIT), showing promising therapeutic properties, such as immunogenicity, tumor homing and tropism towards malignant B cells; more importantly, PVX showed delayed tumor progression in a murine melanoma tumor model and improved IIT in combination with chemotherapy.

Using a murine diffuse large B cell lymphoma model (A20 lymphoma), we demonstrated PVXs efficacy as an IIT in treating tumors as well as preventing recurrence upon re-challenging studies. While plant viruses' mechanism of action is yet to be fully understood, our data provides insights into immunological pathways for CPMV and PVX efficacy through histology and confocal analysis. We envision that repurposing viral nanotechnologies may play a role in more efficacious cancer immunotherapy strategies, preventing relapses and eliciting durable systemic antitumor immunity.

### Materials and Methods

### Preparation of VNPs and Cy5-Conjugated VNPs (CPMV and PVX)

CPMV was obtained by mechanical inoculation of Vigna unguiculata plants (black-eyed pea No. 5) followed by isolation and purification as previously reported. PVX was isolated from the infected Nicotiana benthamiana leaves as previously described and purified by ultracentrifugation in a 10-45% (w/v) sucrose gradient at 104,000×g for 75 min at 4° C. Cyanine-5 succinimide ester (NHS-sulfo-Cy5; Lumiprobe) was conjugated to CPMV's and PVX's surface lysine residues by N-hydroxysuccinimide (NHS) chemistry as previously described. Briefly, 900 molar excess of Sulfo-Cy5 NHS ester was reacted with CPMV (2 mg/mL in 10 mM potassium phosphate buffer (KP) pH 7) for 2 h at room temperature. PVX (2 mg/mL in 0.1 M KP, pH 7) was mixed with Sulfo-Cy5 NHS ester with a dye to coat protein ratio of 5:1 overnight at room temperature. Following the overnight inversion, Cy5-CPMV and Cy5-PVX were purified by ultracentrifugation at 52,000 rpm (1 h, 4°° C.) with a 30% sucrose gradient The pellet was resuspended using an orbital shaker overnight (4°° C.) in 10 mM KP pH 7. All purified VNPs were kept at 4° C. for further characterization.

### UV-Vis

NanoDrop 2000 Spectrophotometer (Thermo Fisher Scientific) was used to obtain the concentration of CPMV and PVX, and the degree of Cy5 labeling to these VNPs using Beer-Lambert Law (A₂₆₀=εcl), where & is the extinction coefficient, c is the concentration (mg/mL), and 1 is the path length of the spectrophotometer (0.1 cm). CPMV has an ε_{260 nm}=8.1 mL·mg⁻¹·cm⁻¹, PVX has an ε_{260 nm}=2.97 mL·mg⁻¹·cm⁻¹, and Cy5 has an ε_{647 nm}=271000 M⁻¹·cm⁻¹.

### Agarose Gel Electrophoresis

CPMV and Cy5-CPMV (10 μg) were mixed with 6× Gel Loading Purple dye (Biolabs), loaded onto a 1.2% (w/v) agarose gel stained with GelRed® (Gold Biotechnologies) in TAE buffer, and ran for 30 min (120 V and 400 mA). The gel was imaged with a ProteinSimple FluorChem R imaging system under MultiFluor Red channel (607 nm excitation) to image Cy5 fluorescent dye, followed by a UV light image to visualize RNA, and further stained with Coomassie Brilliant Blue G-250 (0.25% w/v) followed by imaging under white light to visualize the protein.

### SDS-Gel Electrophoresis

To confirm Cy5 labeling on CPMV and PVX coat proteins, 10 μg of CPMV and Cy5-CPMV were loaded on precast NuPAGE 4-12% Bis Tris Protein Gel (Invitrogen), while PVX and Cy5-PVX were loaded on precast NuPAGE 12% Bis Tris Protein Gel (Invitrogen). Briefly, VNPs and their fluorescent counterparts were mixed with 4× Lithium Dodecyl Sulfate (LDS) sample buffer (Life Technologies) and NuPAGE™ Sample Reducing Agent (Invitrogen), followed by denaturation (95° C. for 5 minutes) prior to loading onto the SDS-PAGE precast gels. Electrophoresis was conducted at 200 V for 40 min using MOPS buffer (Thermo Fisher Scientific), followed by imaging under MultiFluor Red channel (607 nm excitation; to image the Cy5 label), and staining with GelCode™ Blue Safe protein followed by imaging under white light to detect the protein.

### Size Exclusion Chromatography (SEC)

All purified particles (0.5 mg/mL) were analyzed using ÄKTA™ pure Fast Protein Liquid Chromatography system (GE Healthcare LifeSciences). Samples were eluted through Superose 6 increase 100 GL column at 0.5 mL min⁻¹ flow rate using an isocratic elution profile and with absorbance detectors fixed at 260 nm (nucleic acid), 280 nm (protein), and 647 nm (Cy5).

### Transmission Electron Microscopy (TEM)

CPMV/Cy5-CPMV (0.1 mg/mL in DI H₂O) or PVX/Cy5-PVX (0.5 mg/mL in DI H₂O) were absorbed on Formvar carbon film coated TEM supports with 400-mesh hexagonal copper grids (Electron Microscopy Sciences). PELCO casiGlow operating system was used to render the TEM grids a more hydrophilic surface for VNPs adherence. After samples incubation, grids were washed 2× with DI water followed by staining with 2% (w/v) uranyl acetate (Agar Scientific). The samples were imaged using FEI Tecnai Spirit G2 BioTWIN TEM at 80 kV.

### Dynamic Light Scattering (DLS)

CPMV and Cy5-CPMV (0.2 mg/mL) hydrodynamic diameter was measured using Zetasizer Nano ZSP/Zen5600 (Malvern Panalytical). To calculate particle diameter, weighted mean of the intensity distributions was used after 3 measurements (25° C.).

### Cell Culture and A20 Lymphoma Tumor Model

All animals used in this study were seven to eight weeks old female Balb/c mice obtained from The Jackson Laboratory (Strain #000651). All animal experiments were carried out in accordance with University of California San Diego's Institutional Animal Care and Use Committee (IACUC). Mice were pre-immunized subcutaneously (s.c.) behind the neck with CPMV, PVX, or sterile PBS (n=10; 100 μg/200 μL of sterile PBS; Corning, 21-040-CV). Pre-immunizations followed a prime-boost biweekly schedule, and blood was collected through retro-orbital bleeding (using lithium-heparin-treated tubes (Thomas Scientific)) before the first immunization (Week 0) and then on weeks 2 and 4 post-immunization. Mice plasma was collected by centrifugation (2,000×g/10 min/4° C.) and kept at −20° C. until use.

A20 murine B cell lymphoma cells (ATCC) were cultured at 37° C. (5% CO₂) in Roswell Park Memorial Institute (RPMI) 1640 Medium (Gibco), supplemented with 10% (v/v) inactivated-fetal bovine serum (FBS, Gibco), 1% (v/v) penicillin-streptomycin (Thermo Fisher Scientific) and 0.05 mM 2-mercaptoethanol (Gibco). Two weeks post last immunization, A20 cells were intradermally inoculated (i.d.) into the right flank of the mice (200,000 cells in 30 μL of sterile PBS) and mice were monitored every other day for tumor progression. When tumors reached 30 mm³ (˜7-9 days post-inoculation), CPMV or PVX (n=10; 100 μg/20 μL of sterile PBS) were administered intratumorally (i.t.) 3 times, weekly. PBS (n=10; 20 μL) was used as control. Survivors were rechallenged 10 weeks post tumor inoculation (˜5-6 weeks of tumor remission). A20 cells were intradermally inoculated (i.d.) into the left flank of the survivors (200,000 cells/30 μL of sterile PBS), and the mice were monitored every other day for tumor progression; age-matched (n=8) naïve mice were used as a control.

### Immunofluorescence Staining

To further investigate the tumor microenvironment after CPMV or PVX IIT injections, another set of seven to eight weeks old female Balb/c mice (n=5 for CPMV and PVX; n=3 for PBS) were pre-immunized following the same schedule described above. A20 cells (200,000 cells in 30 μL of sterile PBS) were administered intradermally into the right flank. Mice were monitored every two days for signs of tumor progression. Once the tumors reached 30 mm³, the animals received one i.t. injection of Cy5-CPMV or Cy5-PVX (N=5, 100 μg in 20 μL of PBS). PBS (n=3; 20 μL) was used as a control. Treated tumors were harvested 24 or 72 hours post i.t. injection and snap frozen using liquid nitrogen. Frozen tumors were embedded in optimal cutting temperature medium (O.C.T., Fisher Healthcare) and sliced into 10 μm thick sections using the Leica CM1860 cryostat. Tissue sections were then mounted on Superfrost™ Plus Microscope Slides (Fisherbrand) for immunofluorescence (I.F.) staining.

For I.F. staining, the O.C.T. was first washed off from the slides by treating them with 1×PBS for 5 minutes. The tumors were fixed with 4% paraformaldehyde (PFA) in 1×PBS for 10 minutes at room temperature (RT). The slides were then washed 3 times with 1×PBS for 5 minutes, followed by a blocking solution of 1% (w/v) BSA in 1×PBS with 0.05% (v/v) Tween 20 (PBST) for 1 hour at RT. Tumors were washed 3 times with 1×PBS for 5 minutes, while the primary antibodies were prepared in 1% (w/v) BSA in 1×PBST. Primary antibody staining was carried out overnight at 4° C. in a humidified chamber. The primary antibodies consisted of rabbit anti-mouse CD19-1:50 (Novus Biologicals Cat #NBP2-15782), rat anti-mouse Ly-6G/Ly-6C-1:100 (Invitrogen Cat #14-5931-82), and rat anti-mouse F4/80-1:100 (Invitrogen Cat #MA5-16624). The secondary antibodies (Alexa Fluor™ 555 goat anti-rabbit IgG-(Invitrogen Cat #A-21428) and Alexa Fluor™ 555 goat anti-rat IgG-1:1000 (Invitrogen Cat #A-21434)) were prepared in 1×PBST with 1% BSA and stained for 1 hour at RT in the dark following 3 washes with 1×PBS. The slides were once again washed 3 times with 1×PBS, and mounted using Fluoroshield™ with DAPI histology mounting media (Sigma-Aldrich) for 5-10 minutes at RT. The tissues were sealed with 12 mm circular cover glass (Electron Microscopy Sciences) and nail polish (Electron Microscopy Sciences). Once the mounting media was dry, the slides were stored at 4° C. and imaged on the Nikon AIR Confocal/TIRF STORM confocal microscope.

### Detection of Anti-VNPs Antibodies in Mice

Anti-CPMV and anti-PVX specific IgG antibody titers in mice plasma were detected through 2-fold serial plasma dilutions by ELISA (Enzyme-Linked Immunosorbent Assay) as previously described. The signal was developed with 1-Step Ultra TMB-ELISA substrate solution (3,3′,5,5′-tetramethylbenzidine, Thermo Fisher Scientific) and quenched with 2N sulfuric acid (Spectrum Chemical). The absorbance was read at 450 nm using an Infinite 200 Pro microplate reader and i-control software (Tecan, Männedorf, Switzerland).

### Flow Cytometry

VNP cellular uptake by Raw 264.7 macrophages or A20 murine B cell lymphoma cells was tested in the presence or absence of naïve (Week 0) or immunized (Week 4) mice plasma using Cy5-labeled CPMV or PVX (Cy5-CPMV or Cy5-PVX). Raw 264.7 cells (ATCC) were maintained at 37° C. (5% CO₂) in Dulbecco's Modified Eagle's Medium (DMEM) supplemented with 10% (v/v) heat-inactivated fetal bovine serum and 100 units/mL penicillin. A20 murine B lymphoma cells (ATCC) were maintained at 37° C. (5% CO₂) in RPMI-1640 supplemented with 10% (v/v) fetal bovine serum and 100 units/mL penicillin and 100 g/mL Streptomycin and 0.05 mM 2-mercaptoethanol. Cy5-CPMV or Cy5-PVX (1 μg/50 μL serum-free DMEM/well) was preincubated with mice plasma (1:200 dilutions) or serum-free DMEM in a 96-well v-bottom plate at room temperature for 20 minutes. Then, Raw 264.7 or A20 cells were added to the wells (200,000 cells/50 μL serum-free DMEM/well), and the plate was incubated (37° C., 5% CO₂) for 2 h or 8 h. Plate was then centrifuged (500×g/4 min/4° C.) and washed one time with cold sterile PBS and once with cold sterile BD Pharmingen™ Stain Buffer (FBS) (Cat No. 554656). Cells were then fixed with 4% paraformaldehyde (EM grade 15714-S, diluted in PBS; 100 μL/well) for 10 min on ice, followed by 100 μL/well of Stain Buffer to dilute the 4% PFA. The cells were washed once, resuspended on Stain buffer (100 μL/well), and kept in 4° C. until they were analyzed using a BD Acuri C6 Plus flow cytometer. The data was analyzed using the FlowJo v8.6.3 software.

### RAW-Blue™ Cell Activation Assay

RAW-Blue™ assay was performed as per manufacturer's recommendation (Invivogen). Briefly, Raw-Blue cells (100,000 cells/200 μL media per well) were seeded in a 96-well flat-bottom plates, followed by triplicates of CPMV and PVX (1, 5, 10 and 20 μg/well), a negative control (cells with test media), and a positive control of 1×lipopolysaccharide (5 μg/mL LPS, Thermo Fisher Scientific). The same VNP concentrations were also tested in the presence and absence of naïve (Week 0) or immunized (Week 4) mice plasma. After 24 h incubation (37° C., 5% CO₂), 20 μL of supernatant from each well was incubated with 180 μL of QUANTI-Blue solution (Invivogen) and incubated (37° C., 5% CO₂) for 6 h. Absorbance was read at 655 nm using an Infinite 200 Pro microplate reader and i-control software (Tecan, Männedorf, Switzerland).

### Abeomics (Abeomics Inc., CA, USA) Custom-Made TLR7 Agonist Assay

Abeomics Inc., CA, USA performed a custom-made TLR7 agonist assay testing both CPMV and PVX using a TLR7/NF-kB Luc reporter-HEK 293 cells according to manufacturer's protocol. Briefly, cells were plated in 96-well white solid bottom plates for 16 h (37° C., 5% CO₂) in using DMEM medium (w/L-Glutamine, 4.5 g/L Glucose and Sodium Pyruvate) supplemented with 10% heat-inactivated FBS and 1% Pen/Strep. Cells were stimulated with a series of CPMV or PVX concentration (0.03, 0.1, 0.3, 1, 3, 10, 30 and 100 ug/mL), in triplicate for another 16 h (37° C., 5% CO₂). Ebola GP2 protein (R848) was used as a positive control in the same concentration as the VNPs. Luciferase activity was measured and analyzed by Abeomics Inc., CA, USA. Further, CPMV and PVX were packed with lipofectamine vehicle to enhance internalization of the samples, and then tested as described above.

### Results and Discussion

### Vnps Characterization

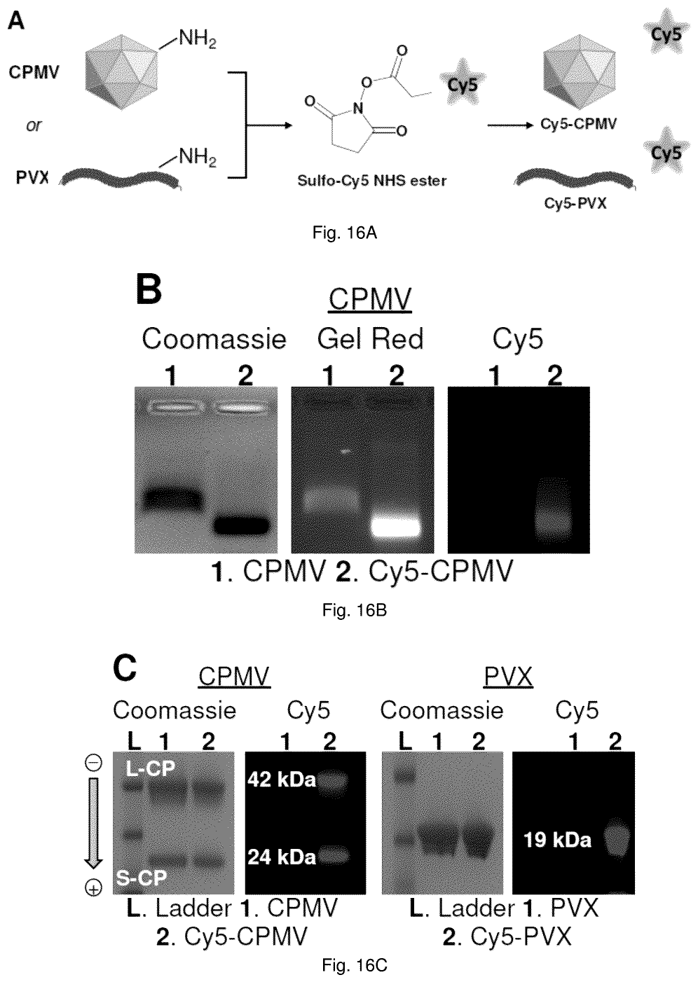

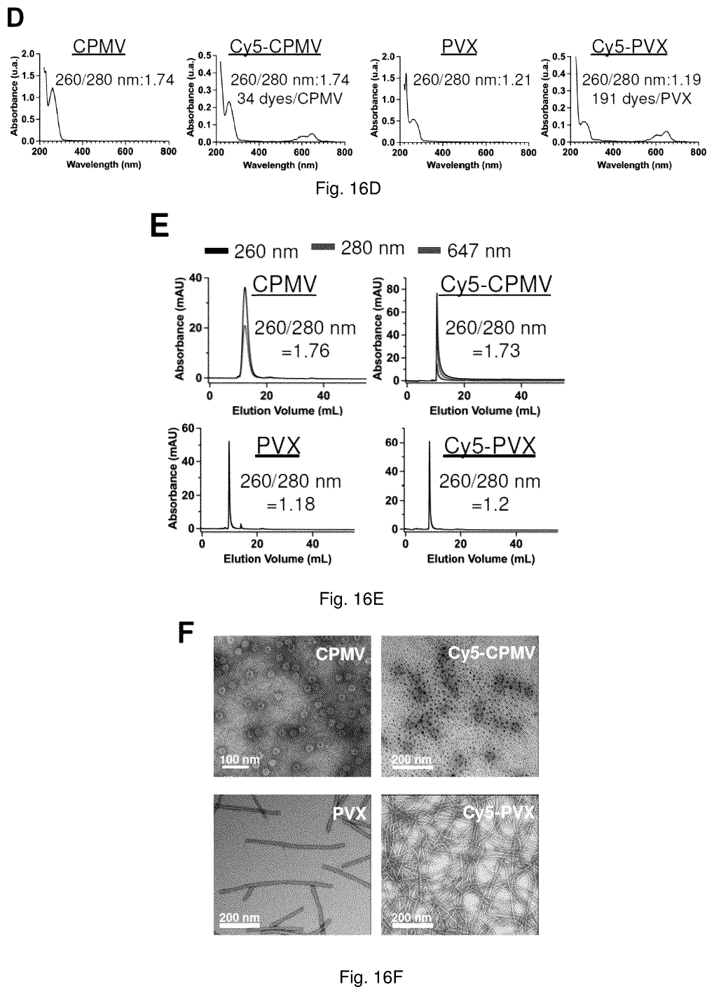

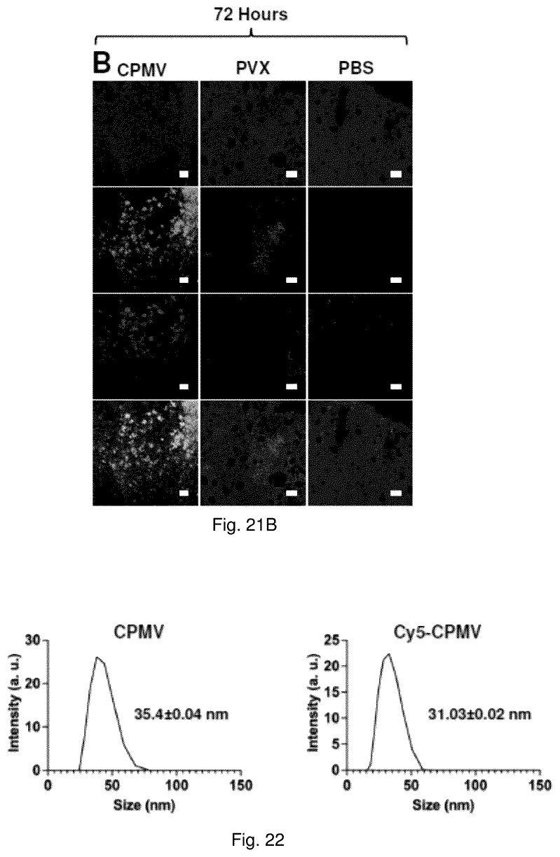

Native and Cy5-conjugated CPMV or PVX were purified and characterized. Solvent exposed lysine residues from CPMV or PVX were used to conjugate with sulfo-Cy5 through N-hydroxysuccinimide (NHS) chemistry, FIG. 16A. Native and denaturing gel electrophoresis confirmed successful dye conjugation and viral coat protein integrity with intact particles and no free dye, FIG. 16B, C. Native agarose gel electrophoresis revealed that CPMV RNA colocalizes with the coat proteins and dye (Cy5-CPMV), FIG. 16B. PVX was not analyzed by agarose gel as the native PVX, with its 515 nm length, exceeds the pore size of the 1.2% (w/v) gel and thus does not run through. Denaturing sodium dodecyl sulfate polyacrylamide gel electrophoresis (SDS-PAGE) showed both small (S-CP; ˜24 kDa) and large (L-CP; ˜42 kDa) coat proteins for CPMV, while PVX revealed only one coat protein (˜19 kDa), FIG. 16C. Fluorescence signal of the SDS gel colocalizes with the respective coat proteins, confirming the dye-labeling of CPMV and PVX, FIG. 16C. UV-Vis was used to calculate the degree of Cy5 labeling in both VNPs with Beer-Lambert Law and the extinction coefficients at 260 nm (ε_{cpmv}=8.1 mL·mg⁻¹·cm⁻¹, ε_{PVX}=2.97 mL·mg⁻¹·cm⁻¹ and ε_{cy5}=271000 M⁻¹·cm⁻¹), FIG. 16D. About 34 Cy5 dye molecules were conjugated onto each CPMV particle while ˜191 Cy5 dyes were conjugated onto each PVX particle; PVX is composed of 1270 identical coat proteins (25 kDa), presenting 11 lysine residues in each coat protein subunit compared to the 300 solvent-exposed lysine residues of CPMV, resulting in the difference of label degree between these VNPs. Size exclusion chromatography (SEC), transmission electron microscopy (TEM) and dynamic light scattering (DLS) were used to confirm particle integrity and homogeneity. The elution profiles of CPMV and PVX coordinate with their conjugated counterparts with the characteristic peaks at ˜10-12 mL and 9 mL, respectively, FIG. 16E. The A260 to A280 ratio are conserved before and after dye conjugation, indicating that particles remained intact and non-aggregated. The colocalization of the elusion peak with 280 nm and 647 nm absorbance indicate the successful Cy5 conjugation. No free dye was detected as the characteristic peaks at ˜11 mL and 9 mL for CPMV and PVX, respectively, are the only peaks with 647 nm absorbance. TEM images further confirmed the intact icosahedral CPMV structure and filamentous PVX structure, regardless of dye conjugation, FIG. 16F. DLS corroborates with this data, revealing a hydrodynamic diameter of ˜30 nm with a narrow polydispersity index (PDI) of 0.1 for CPMV, FIG. 22. Since DLS analysis measures Stokes radius, which assume a spherical shape, it was not performed on the filamentous PVX samples.

### Anti-Tumor Efficacy of CPMV and PVX

For a translational point of view, preexistence of immunity is a common concern in clinical trials of viral particles drug candidates, as it can impact efficacy and dosage. For instance, preexistence of specific anti-PEG antibodies lead to premature cargo release and rapid clearance of PEGylated lipid nanoparticles, mainly through the activation of the complement system. Similarly, preexistence of neutralizing antibodies poses a hurdle for therapeutic efficiency of OVs. It has been reported that humans have antibodies against plant virus likely related to distribution and food consumption; however, we have previously shown that anti-CPMV specific antibodies enhance its efficacy as an intratumoral immunotherapy (IIT). Rather than neutralizing, these specific antibodies seems to potentiate the immunostimulatory properties of the CPMV, increasing opsonization leading to a higher uptake by antigen presenting cells. Therefore, in this study, we investigate whether preexisting immunity against PVX would result in similar anti-tumor efficacy as observed for CPMV.

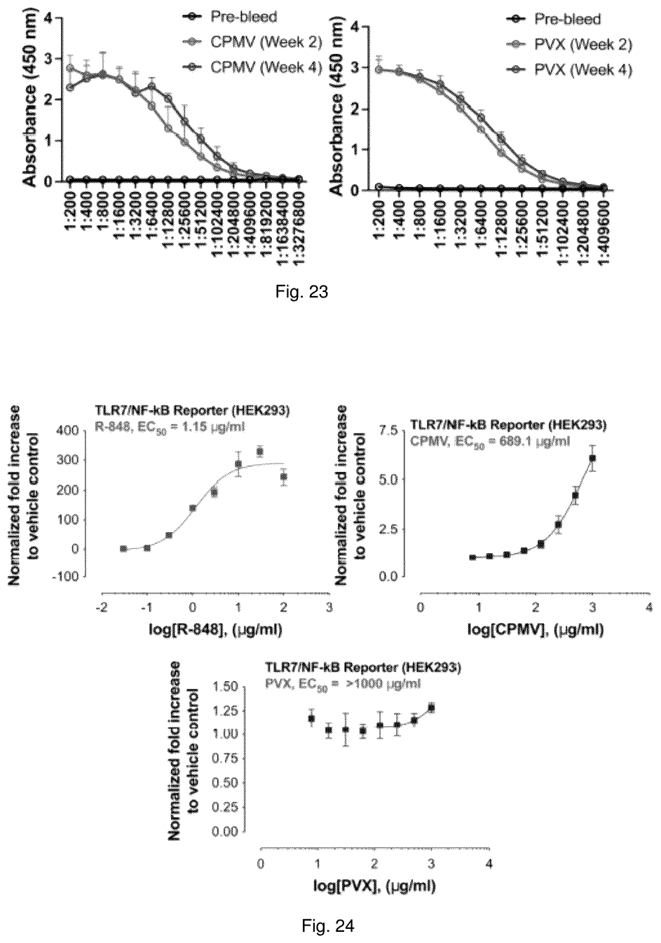

Prior to tumor inoculation, we preimmunized female Balb/c mice (n=10/treatment group) adopting a homologous prime-boost regimen (100 μg of CPMV or PVX were injected s.c. behind the neck, biweekly at weeks 0 and 2). Blood was harvested (at the beginning of weeks 2 and 4) and plasma was screened for anti-CPMV and anti-PVX specific antibodies using ELISA, FIG. 23. As expected, CPMV elicited 4-fold higher anti-CPMV antibody titers (endpoint titer˜1,638,400) when compared to the anti-PVX titers (endpoint titer˜409,600). Previous studies comparing these two VNPs have shown that CPMV is endocytosed more efficiently by dendritic cells which leads to improved antigen processing and transport to draining lymph nodes (dLNs). CPMV is retained in the dLNs for a longer period of time which allows prolonged immune cell interaction. Once in the dLNs, the antigens are presented to naive B cells in the germinal centers within the lymphoid organs which initiates the humoral immune response. However, larger particles such as PVX drain more slowly and traffic to dLNs less efficiently, leading to a lower immunogenicity when compared to CPMV.

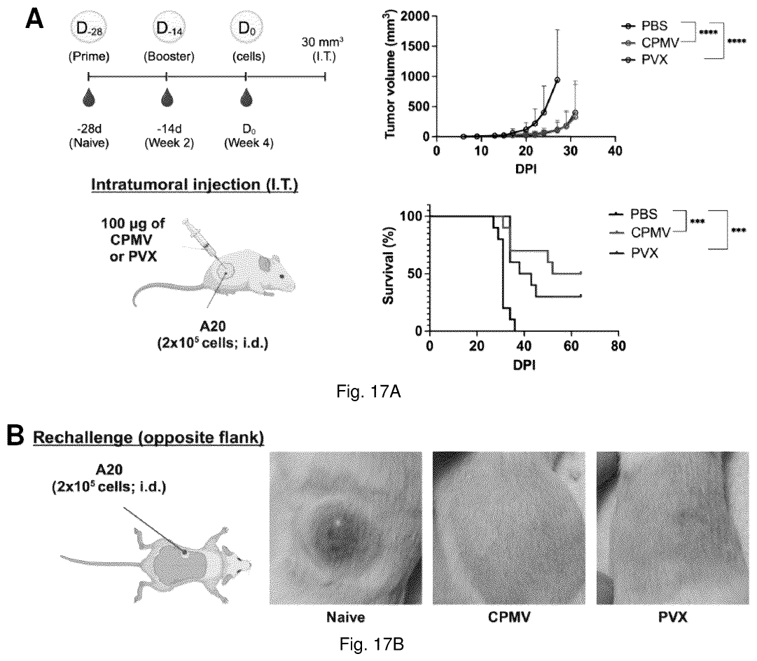

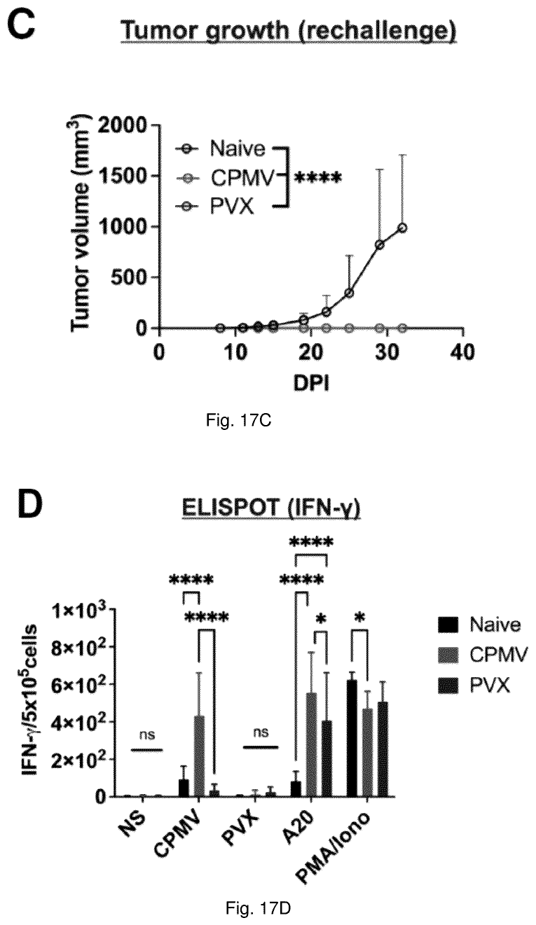

Mice were inoculated with A20 lymphoma cells intradermally two weeks after the last immunization, and they were treated weekly with intratumoral (IIT) injections of CPMV or PXV (100 μg/20 μL) once the tumors reached ˜30 mm³. A set of mice (n=10) were i.t. injected with PBS (vehicle control), and tumors were measured every other day, FIG. 17A. Survivor tumor-free mice (CPMV-treated n=5, PVX-treated n=3) were monitored for 8 weeks post-last IIT for recurrence. These mice were rechallenged with A20 cells to investigate anti-tumor immune memory, using a cohort of age-matched mice (n=8) as a control, FIG. 17B and 17C. No tumorigenesis was observed in CPMV- and PVX-treated survivor mice, indicating immune memory against A20 cells for 30+ days after tumor rechallenge. On day 32 post tumor rechallenge, spleens of the survivors and controls were harvested and prepared for ELISPOT assay.

Splenocytes were stimulated with CPMV, PVX and A20 cells to further corroborate immune memory. FIG. 17D shows that CPMV stimulation upregulates the production of IFN-γ while PVX does not; this highlights CPMV's superior ability to stimulate the immune system. Most importantly, we can see that stimulation with A20 cells elicits IFN-γ secretion for both CPMV and PVX-treated groups. IFN-γ is a proinflammatory cytokine secreted by different immune cells, including Th1 CD4 helper T cells, macrophages, activated CD8 T cells as well as natural killer T cells, among others. Both activated CD4 and CD8 T cell subpopulations have potent anti-tumor properties and are associated with a positive disease prognosis. From these results we can conclude that CPMV and PVX both lead to the establishment of immune memory against A20 cells which explains the lack of recurrence after tumor rechallenge.

### Cellular Uptake and Immunogenicity of CPMV and PVX

To gather more insights on in vivo behavior of the studied VNPs, we conducted some in vitro assays. Here, we compared the uptake of CPMV with PVX particles by macrophages, more specifically Raw 264.7 cells (mouse macrophage cell line); CPMV has an icosahedral shape while PVX is a filamentous virus; Raw 264.7 cells were incubated with fluorescent-labeled CPMV or PVX (Cy5-CPMV or Cy5-PVX) in the presence and absence of immunized mice plasma. Naïve mice plasma (prior to VNPs exposure) was used as control to assess specificity. Uptake assay was performed at 37° C., mimicking the physiological body temperature, to evaluate particles internalization, as opposed to particles-cell interaction/adsorption (usually done at lower temperatures to confers cell membrane rigidity).

Cy5 fluorescence derived from the labeled VNPs was used to measure the internalization extend as a mean fluorescence intensity (MFI, FIG. 18A); VNPs were incubated with naïve and immunized mouse plasma (1:200; to simulate opsonization) for 20 min prior to cell incubation in a serum-free media to minimize interaction with other components of the media that could intervene with on the results. VNPs were then incubated with Raw 264.7 cells for 2 h and 8 h at 37° C., followed by several steps of washing to remove non-internalized VNPs. Both CPMV and PVX uptake is dependent on time and presence of specific anti-VNPs antibodies, as expected. We noticed an 8-fold enhancement on CPMV uptake whenever pre-incubated with immunized plasma (compared to naïve). On the other hand, this enhancement was only about 1.5-fold for PVX. When we compared opsonized CPMV vs PVX (pre-incubated with immunized plasma), we noticed a 2-fold higher uptake of CPMV compared to PVX at 2 h incubation, followed by an 8-fold higher uptake after 8 h incubation.

Phagocytosis is an internalization mechanism intrinsic to professional antigen presenting cells (APCs), such as macrophages, monocytes, dendritic cells (DCs), neutrophils. It takes from 30 min to several hours depending on, not only cell type, but also size, shape and surface composition of particles. In vivo, nanoparticles are likely to undergo opsonization by specific antibodies, produced over multiple administrations; the antibodies Fc region can then be recognized by Fc receptor found on the surface of APCs, leading to a higher internalization of NPs.

Data indicates that elongated particles may evade uptake by macrophages, leading to a prolonged circulation in vivo, while spherical small nanoparticles can be rapidly uptake, justifying the difference in CPMV vs PVX uptake by Raw 264.7 macrophages.

To further investigate the immunogenicity of CPMV and PVX, QUANTI-Blue assay was used. RAW-Blue™ mouse macrophages cells (derived from Raw 264.7 murine cells) express a variety of pattern recognition receptors (PRRs) such as the toll-like receptors (TLRs), NOD 1 and 2 receptors, RIG-1 as well as C-type lectin receptors; upon stimulation, AP-1 and NF-κB transcription factors leads to secretion of an engineered reporter gene, embryonic alkaline phosphatase (SEAP), in the cell culture media that can be detected by QUANTI-Blue assay (Invivogen). We screened different concentration of VNPs (1, 5, 10 and 20 μg VNPs/100,000 RAW-Blue™ cells for 24 hours). Bacterial lipopolysaccharide (LPS, 1×, Invitrogen) was used as positive control, while non-stimulated cells served as negative control (FIG. 18B). We also investigate if the SEAP secretion would change when VNPs were incubated with naïve or immunized plasma prior to cells incubation. The proteinaccous structure of VNPs act as pathogen-associated molecular pattern (PAMPs) that can be recognized by the cells PRRs; We noticed the regardless of specific anti-PVX presence, the levels of SEAP secreted upon all PVX-tested conditions are similar to the cell control (negative control), likely related to the lower macrophage uptake. However, CPMV showed a concentration-dependent activation of RAW-Blue™ cells (˜2-fold difference between concentrations) that was also affected by the presence of naïve or anti-CPMV specific antibodies (slightly increased), indicating that opsonization effect of antibodies leads to a higher macrophage uptake, enhancing the immunogenicity of CPMV.

To further delineate a possible explanation for the comparable anti-tumor effect of PVX, we sought to investigate TLR7 activation signaling by CPMV and PVX, through a custom made TLR7/NF-kB Luc reporter cell line assay (Abcomics Inc., CA, USA). It has been described that CPMV anti-tumoral potency is likely related to its multivalent interaction with immune cells; albeit non-infectious towards mammals, CPMV can be recognized by PRRs, especially TLRs; the protein capsid stimulates TLRs 2 and 4, while its viral ssRNA serves as an agonist to TLR7, leading to activation of innate immunity; cross-talking between innate and adaptive immune system can elicit long-lasting systemic and immune memory. Filamentous viruses have been considered as potential candidates for cancer therapy; their high aspect ratio allow prolonged circulation time, evading phagocytosis that leads to an enhanced tumor accumulation. For instance, papaya mosaic virus (PapMV) had shown potential anti-tumor response as a IIT candidate, promoting tumor delayed progression associated with a significantly increased pro-inflammatory cytokines released upon TLR7 stimulation by PapMV ssRNA.

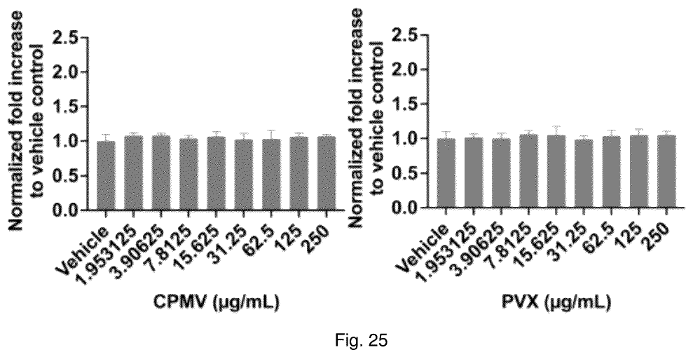

Contrasting PapMV immune response, our data suggests that TLR7 signaling might be not the only mechanism for anti-tumor efficacy for the VNPs here studied; surprisingly CPMV showed a moderate TLR7 agonist effect (EC₅₀=689 μg/mL; FIG. 24) profile compared to positive control R848 (FIG. 18C), while PVX barely agonized TLR7 (EC₅₀>1000 μg/mL; FIG. 24) in the TLR7/NF-kB Luc reporter cell line, suggesting we suggest that more than one immunologic pathway is associated with IIT efficacy for both VNPs here studied. These results were confirmed by packing CPMV and PVX with lipofectamine, vehicle to enhance internalization bypassing the endosome; TLR7 is found in the endosome and CPMV moderate agonist effect was confirmed as no signal was detected (regardless of concentration used) when packed with lipofectamine (FIG. 25). Same results were demonstrated for PVX, corroborating with previous results.

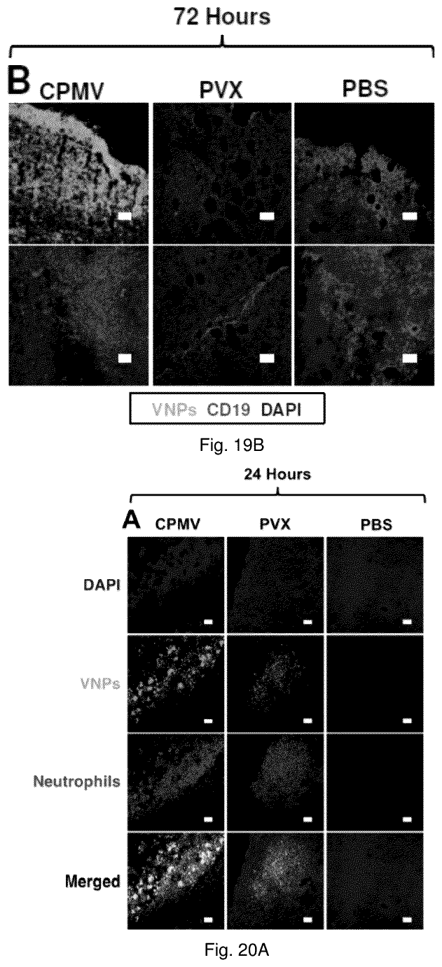

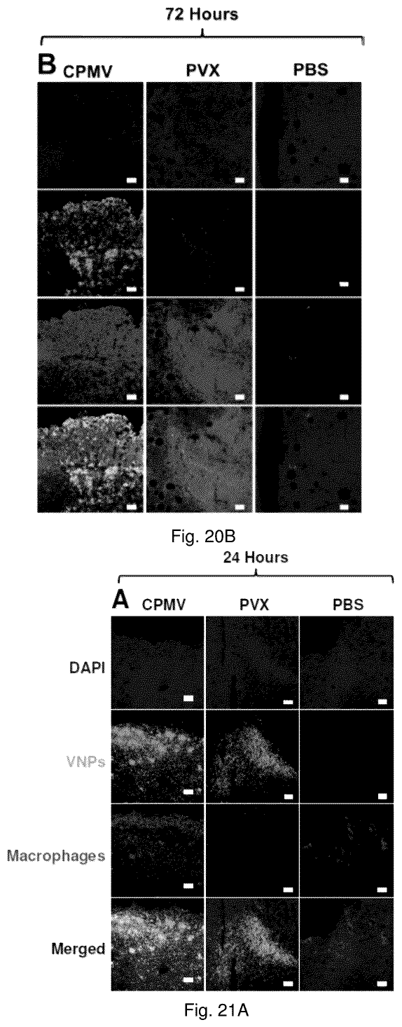

We have previously demonstrated that empty CPMV (eCPMV; devoid RNA) have similar anti-tumor efficacy as CPMV, eliciting systemic immune response in both mouse tumor models and canine patients. We speculate that PVX anti-tumor immune response is likely related to its proteinaceous highly structured nature and ability to recruit innate immune cells, mostly neutrophils, into the TME (FIG. 20), leading to a robust cellular and humoral immune response; further experiments are needed to better understanding PVX immune response. Delineating the ITT mechanism of these two distinct VNPs was out of the scope of this paper as we decided to follow a different direction in evaluate the potential of different VNPs as immunotherapy candidates.

### Intratumoral VNPs in Preimmunized Animals Clear B Cell Lymphoma Cells

Given that we used the A20 B cell lymphoma tumor model, we sought to investigate the effect our treatment had on B cell populations within the TME. We have previously reported that intraperitoneally administered PVX has a natural tropism to B lymphoma cells, while CPMV is described to preferentially interact with other APCs, mostly DCs. We utilized immunofluorescence (IF) staining followed by confocal microscopy to further understand how our VNPs were interacting within the tumor microenvironment. Similar to the in vivo efficacy studies, we preimmunized female Balb/c mice with CPMV or PVX (100 μg, s.c. behind the neck) in a biweekly homologous prime-boost regimen. A20 tumors were inoculated intradermally two weeks post-last immunization; mice were administered with one IIT of Cy5-labeled VNPs (100 μg, Cy5-CPMV or Cy5-PVX) once the tumors reached ˜30 mm³. Tumors were collected 24 or 72 hours post-last IIT, cryosectioned and prepared for IF staining. PBS was used as a vehicle control.

Staining with the anti-mouse CD19 antibody revealed a clear difference between VNP-positive and VNP-negative regions within the tumor, FIG. 19. At 24 hours post IIT, we can see that both CPMV and PVX led to B cells clearance (top row), whereas the TME regions that lacks VNPs have a B cell signal comparable to the PBS control (bottom row), FIG. 19A. The morphology of the tumor itself is visibly different in the VNP-negative regions, showing a denser distribution of cells, suggesting that VNPs are changing the TME (VNP-positive regions). Notably, at this early timepoint it is evident that PVX is rapidly cleared from the tumor compared to the CPMV group. At 72 hours post-IIT, CPMV remains within the tumor while PVX has been mostly cleared (top row), FIG. 19B. However, there was a lower CD19 signal compared to CPMV suggesting a long-lasting effect of PVX even after it is no longer present in TME.

### Intratumoral VNPs in Preimmunized Animals Interact With Tumor-Infiltrating Innate Immune Cells

There are numerous reports of CPMV's ability to recruit innate immune cells following IIT administration. However, little is known about intratumoral immunotherapy using PVX; PVX's interaction with immune cells has been mostly studied in the context of intraperitoneal, intravenous, and subcutaneous administration. For this study, we sought to compare IIT CPMV and PVX using IF staining to corroborate the in vivo efficacy. The first innate immune cell population we investigated were neutrophils using the anti-mouse Ly-6G/Ly-6C primary antibody. Tumor infiltrating neutrophils are crucial in cancer immunotherapy because of their cell killing and immune activation properties. At 24 hours post IIT, we can already notice a strong co-localization with the tumor-infiltrating neutrophils for both particles (FIG. 20A) regardless of PVX rapid clearance; however, the number of neutrophils within the tumor is strikingly similar upon treatment with either VNPs (FIG. 20B). Qualitatively, one could suggest that PVX might recruits neutrophils even more efficiently as CPMV, in accordance with FIG. 19; hence PVX elicits a durable anti-tumor innate immune response within the tumor, likely to be related to its promising efficacy in the in vivo study. It can be hypothesized that PVX compensates for the rapid clearance by leaving behind a strong activated immune cell population; nonetheless, CPMV outperformance is likely related to its prolonged interaction with immune cells in the TME, remaining within the tumor site for longer period of time.

Given our in vitro results using the Raw 264.7 and RAW-Blue™ macrophages, we also investigated how VNPs interacted with macrophages in vivo. Tumor associated macrophages (TAMs) are the most prevalent innate immune cells within the TME; depending on their phenotype, they can delay tumor progression, bridging innate and adaptive immune system, making them an important factor in immunotherapy efficacy. Similar to the previous results, CPMV has a strong retention within the TME and co-localization with macrophages at 24 and 72 hours post IIT (FIG. 21A, B). Of note, macrophages were found in the TME of PBS treated tumors, which could be indicative of an M2 immunosuppressive macrophage population. We have previously shown that CPMV can repolarize M2 macrophages into the cytotoxic M1 phenotype which explains the increase in signal for the CPMV group. However, from these images, one could hypothesized that PVX have subtle interaction with TAMs population at either timepoint (FIGS. 21A and B). This corroborates with the results shown in RAW-Blue™ assay (FIG. 18B); albeit being uptake by Raw 264.7 cells (FIG. 18A), PVX barely co-localize with this immune cell population in vivo. In vitro assay we are promoting the interaction between PVX and macrophages by placing them in a confined space which results in cellular uptake; however in an in vivo model, PVX might show different immune cell tropism and its high aspect ratio may lead to evasion of APCs phagocytosis, leading to a lower antigen presentation, resulting in the observed different performance when compared to CPMV as an immunotherapy agent.

In this example, we have shown that two morphologically distinct plant viruses were able to elicit a durable and systemic antitumor immune response against an aggressive B cell lymphoma tumor model. We and others have reported other plant virus nanoparticles as strong cancer immunotherapy candidates, due to their exquisite potential as immune modulators of the TME. Data suggest that when intratumorally administered, VNPs interact and activate innate immune cells within the TME, through PRR recognition, such as TLRs, restarting the cancer immunity cycle. CPMV outperformance compared to other VNPs IIT is yet to be fully understood, however most likely to be related to its multivalent interaction with immune cells and long retention time within the tumors. Interestingly, PVX showed a similar potent anti-tumor immune response, regardless of their distinct physiochemical properties. Similarly, another filamentous plant virus, Papaya Mosaic Virus (PapMV) showed promising results in murine melanoma model, and its efficacy was attributed to the viral RNA that stimulate TLR7 and leads to a high inflammatory response (strong type I IFN response). However, our data suggest that PVX might activate antitumor response by another immunological pathway, as it showed no TLR7 agonist potential, nor was it able to induce strong IFN-γ response in splenocytes assay. Previous reports showed that viral genome, and consequently TLR7 activation, are not essential for an efficacious antitumor immune response; hence further experiments will provide a better understanding of PVX's mechanism of action and they were out of scope of the present study. Here, we sought to highlight the potential of different VNPs as safe and efficacious cancer immunotherapy candidates, that elicit long-lasting and systemic antitumor immune responses, that could prevent metastasis and recurrence in patients in clinical settings.

The complete disclosure of all patents, patent applications, and publications, and electronically available materials cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. An intratumoral immunotherapy for treating cancer in a subject in need thereof, comprising a therapeutically effective amounts of cowpea mosaic virus (CPMV), CPMV virus-like particles, potato virus X (PVX), and/or PVX virus-like particles formulated for in situ administration to cancer of the subject, wherein the therapeutically effective amount of CPMV, CPMV virus-like particles, PVX, and/or PVX virus-like particles is an amount effective to provide a durable and systemic anticancer response against cancer metastasis and recurrence in the subject, and the CPMV, CPMV virus-like particles, PVX, and/or PVX virus-like particles are not used as a vehicle for drug or antigen delivery.

2. The intratumoral immunotherapy of claim 1, wherein CPMV, CPMV virus-like particles, PVX, and/or PVX virus-like particles are formulated for intratumoral injection.

3. The intratumoral immunotherapy of claim 1, wherein CPMV, CPMV virus-like particles, PVX, and/or PVX virus-like particles are provided in a pharmaceutical composition configured for administration proximal to a tumor in the subject.

4. The intratumoral immunotherapy of claim 1, wherein CPMV, CPMV virus-like particles, PVX, and/or PVX virus-like particles are provided in a formulation with an anti-cancer agent for in situ administration to the subject.

5. The intratumoral immunotherapy of claim 4, wherein the anticancer agent is a chemotherapy or immunotherapy anticancer agent.

6. The intratumoral immunotherapy of claim 4, wherein the anticancer agent comprises monoclonal antibodies.

7. The intratumoral immunotherapy of claim 1, wherein the anti-cancer agent is doxorubicin.

8. The intratumoral immunotherapy of claim 1, wherein the cancer is metastatic cancer.

9. The intratumoral immunotherapy of claim 1, wherein the cancer is selected from the group consisting of melanoma, breast cancer, colon cancer, lung cancer, ovarian cancer, and lymphoma.

10. The intratumoral immunotherapy of claim 1, wherein the cancer is lymphoma.

11. The intratumoral immunotherapy of claim 1, comprising a combination of at least one of CPMV or CPMV virus-like particles and at least one of PVX, and/or PVX virus-like particles.
